SEQUENCE LISTING (35 sequences)

SEQ ID NO: 1 - protein, 586 aa, Homo sapiens
MGPLTFRDVKIEFSLEEWQCLDTAQRNLYRDVMLENYRNLVFLGIAVSKPDLITWLEQGKEPWNLKRHEMVDKTPVMCSHFAQDVWPEHSIKDSFQKVILRTYGKYGHENLQLRKDHKSVDACKVYKGGYNGLNQCLTTTDSKIFQCDKYVKVFHKFPNVNRNKIRHTGKKPFKCKNRGKSFCMLSQLTQHKKIHTREYSYKCEECGKAFNWSSTLTKHKIIHTGEKPYKCEECGKAFNRSSNLTKHKIIHTGEKPYKCEECGKAFNRSSTLTKHKRIHTEEKPYKCEECGKAFNQFSILNKHKRIHMEDKPYKCEECGKAFRVFSILKKHKIIHTGEKPYKCEECGKAFNQFSNLTKHKIIHTGEKPYKCDECGKAFNQSSTLTKHKRIHTGEKPYKCEECGKAFKQSSTLTEHKIIHTGEKPYKCEKCGKAFSWSSAFTKHKRNHMEDKPYKCEECGKAFSVFSTLTKHKIIHTREKPYKCEECGKAFNQSSIFTKHKIIHTEGKSYKCEKCGNAFNQSSNLTARKIIYTGEKPYKYEECDKAFNKFSTLITHQIIYTGEKPCKHECGRAFNKSSNYTKEKLQT

SEQ ID NO: 2 - DNA, 3123 bp, Homo sapiens
  source 1..3123: mol_type = other DNA
ctctcgctgcagccggcgctccacgtctagtcttcactgctctgcgtcctgtgctgataaaggctcgccgctgtgaccctgttacctgcaagaacttggaggttcacagctaagacgccaggaccccctggaagcctagaaatgggaccactgacatttagggatgtgaaaatagaattctctctagaggaatggcaatgcctggacactgcgcagcggaatttatatagagatgtgatgttagagaactacagaaacctggtcttccttggtattgctgtctctaagccagacctgatcacctggctggagcaaggaaaagagccctggaatctgaagagacatgagatggtagacaaaaccccagttatgtgttctcattttgcccaagatgtttggccagagcacagcataaaagattctttccaaaaagtgatactgagaacatatggaaaatatggacatgagaatttacagctaagaaaagaccataaaagtgtggatgcatgtaaggtgtacaaaggaggttataatggacttaaccagtgtttgacaactactgacagcaagatatttcagtgtgataaatatgtgaaagtctttcataaatttccaaatgtaaatagaaataagataagacatactggaaagaaacctttcaaatgtaaaaaccgtggcaaatcattttgcatgctttcacaattaactcaacataagaaaattcatactagagagtattcttacaaatgtgaagaatgtggtaaagcctttaactggtcctcaacccttactaaacataagataattcatactggagaaaaaccctacaaatgtgaagaatgtggcaaagcttttaaccggtcctcaaatcttactaaacataaaataattcatactggagagaaaccctacaaatgtgaagaatgtggcaaagcttttaaccggtcctcaacccttactaaacataaaagaattcatacagaagagaaaccctacaaatgtgaagaatgtggcaaggcctttaaccagttctcgattcttaataaacataagagaattcatatggaagataaaccctacaaatgtgaagaatgtggcaaagcctttagagtattctcaattcttaaaaaacataagataatccatactggggaaaaaccatacaaatgtgaagaatgtggcaaagcctttaaccagttctcaaaccttactaaacataagataattcatactggagagaaaccctacaaatgtgatgaatgtggcaaagcctttaaccagtcctcaacccttactaaacataaaagaattcatacgggagaaaaaccctacaaatgtgaagaatgtggcaaagcttttaaacagtcctcaacccttactgaacataagataattcatactggagagaaaccctacaaatgtgaaaaatgtggcaaggcctttagctggtcctcagcttttactaaacataagagaaatcatatggaagataaaccctacaaatgtgaagaatgtggcaaagcctttagtgtattctcaacccttactaaacataaaataattcatactagagaaaaaccctacaaatgtgaagaatgtggcaaagcctttaaccagtcctcaatttttactaaacataagataattcacactgaagggaaatcctacaaatgtgaaaaatgtggcaatgcttttaaccagtcctcaaaccttactgcacgtaagataatttatactggagagaaaccctacaaatatgaagaatgtgacaaagcctttaacaagttctcaacccttattacacatcagataatttatactggagagaaaccctgcaaacatgaatgtggcagagcctttaacaaatcctcaaattatactaaagagaaactacaaacctgaaagatgtgacaatgattttcactacacctcaaacttttctaaacataaaccatattggtgccctagaaatgtgaggaatatgacaaggactttaaatggttgtcacgcttgattgtaggtaagataatttatattggagaaaaatcctccaagtatgaagaatgtggcaaacttttaaccaatcctcacaccttattgcacaggaaagcatttatacttgagaaaaattgtataaagaatatggaaaagccatttatatctgctcacatgtaaaaacatcagttcatacttaataaaatgcaattaccgtcaaatctttcagaaaatataagcctttaatacgaggaagagtattcttaagatgaacattacaaatagaaagagggttgtagtacctttagttttatgatagatcttattgtacacattttgtaccagaggaaaaccctaaagcattagttgctcaaactttgttcgacatcagggaatttgtattggagaaaaaccctgcaaatgtaataaatatggaaaaacattttttcaaaaactacagcttggaaaacatcagagagttcatactaaaatatatttttgcagatgcagtaaatatgaaaaatatttaatcccaaattaagtctatgtaaatatcagaattcacagtagaaatcataaggcataaggcactgatacttcagacattacactaaattagagtgttgagtataggagatccaaaactaaaattgttaggtaagttatttatatataactttaaaagaagtagaagattttttggagatttataattacattcaaagtatacttttttcttgaaaaaaattacagattttttgaaaagcaattgatgtaatttaactctcaaattcatgtttttcttcattcctattatattcacatgtgaaagcaagtgatctgttgttgctgaatcagagatatgagagattcttttttataggtgggcattatttatgcccctttctgtggaagagtaagaaaattaaaatacaagatgcatgaggaaaatgtagagatgctctttgtgattaacttagaatattaagtgctacttgacgtacatgttcagactaacattcttttgcagtatagtgagaaaaaaacattttaaaattaattatcattttgttgattgtgcttttatgtaataaaatgcagtactttaaaacaaaaaaaaaaaaaaaa

SEQ ID NO: 3 - protein, 478 aa, Homo sapiens
MDTSDLFASCRKGDVGRVRYLLEQRDVEVNVRDKWDSTPLYYACLCGHEELVLYLLANGARCEANTFDGERCLYGALSDPIRRALRDYKQVTASCRRRDYYDDFLQRLLEQGIHSDVVFVVHGKPFRVHRCVLGARSAYFANMLDTKWKGKSVVVLRHPLINPVAFGALLQYLYTGRLDIGVEHVSDCERLAKQCQLWDLLSDLEAKCEKVSEFVASKPGTCVKVLTIEPPPADPRLREDMALLADCALPPELRGDLWELPFPCPDGFNSCPDICFRVAGCSFLCHKAFFCGRSDYFRALLDDHFRESEEPATSGGPPAVTLHGISPDVFTHVLYYMYSDHTELSPEAAYDVLSVADMYLLPGLKRLCGRSLAQMLDEDTVVGVWRVAKLFRLARLEDQCTEYMAKVIEKLVEREDFVEAVKEEAAAVAARQETDSIPLVDDIRFHVASTVQTYSAIEEAQQRLRALEDLLVSIGLDC

SEQ ID NO: 4 - protein, 693 aa, Homo sapiens
MTPQSLLQTTLFLLSLLFLVQGAHGRGHREDFRFCSQRNQTHRSSLHYKPTPDLRISIENSEEALTVHAPFPAAHPASRSFPDPRGLYHFCLYWNRHAGRLHLLYGKRDFLLSDKASSLLCFQHQEESLAQGPPLLATSVTSWWSPQNISLPSAASFTFSFHSPPHTAAHNASVDMCELKRDLQLLSQFLKHPQKASRRPSAAPASQQLQSLESKLTSVRFMGDMVSFEEDRINATVWKLQPTAGLQDLHIHSRQEEEQSEIMEYSVLLPRTLFQRTKGRSGEAEKRLLLVDFSSQALFQDKNSSQVLGEKVLGIVVQNTKVANLTEPVVLTFQHQLQPKNVTLQCVFWVEDPTLSSPGHWSSAGCETVRRETQTSCFCNHLTYFAVLMVSSVEVDAVHKHYLSLLSYVGCVVSALACLVTIAAYLCSRVPLPCRRKPRDYTIKVHMNLLLAVFLLDTSFLLSEPVALTGSEAGCRASAIFLHFSLLTCLSWMGLEGYNLYRLVVEVFGTYVPGYLLKLSAMGWGFPIFLVTLVALVDVDNYGPIILAVHRTPEGVIYPSMCWIRDSLVSYITNLGLFSLVFLFNMAMLATMVVQILRLRPHTQKWSHVLTLLGLSLVLGLPWALIFFSFASGTFQLVVLYLFSIITSFQGFLIFIWYWSMRLQARGGPSPLKSNSDSARLPISSGSTSSSRI

SEQ ID NO: 5 - protein, 460 aa, Homo sapiens
MAGYLSESDFVMVEEGFSTRDLLKELTLGASQATTDEVAAFFVADLGAIVRKHFCFLKCLPRVRPFYAVKCNSSPGVLKVLAQLGLGFSCANKAEMELVQHIGIPASKIICANPCKQIAQIKYAAKHGIQLLSFDNEMELAKVVKSHPSAKMVLCIATDDSHSLSCLSLKFGVSLKSCRHLLENAKKHHVEVVGVSFHIGSGCPDPQAYAQSIADARLVFEMGTELGHKMHVLDLGGGFPGTEGAKVRFEEIASVINSALDLYFPEGCGVDIFAELGRYYVTSAFTVAVSIIAKKEVLLDQPGREEENGSTSKTIVYHLDEGVYGIFNSVLFDNICPTPILQKKPSTEQPLYSSSLWGPAVDGCDCVAEGLWLPQLHVGDWLVFDNMGAYTVGMGSPFWGTQACHITYAMSRVAWEALRRQLMAAEQEDDVEGVCKPLSCGWEITDTLCVGPVFTPASIM

SEQ ID NO: 6 - protein, 211 aa, Homo sapiens
MQRTGGGAPRPGRNHGLPGSLRQPDPVALLMLLVDADQPEPMRSGARELALFLTPEPGAEAKEVEETIEGMLLRLEEFCSLADLIRSDTSQILEENIPVLKAKLTEMRGIYAKVDRLEAFVKMVGHHVAFLEADVLQAERDHGAFPQALRRWLGSAGLPSFRNVECSGTIPARCNLRLPGSSDSPASASQVAGITEVTCTGARDVRAAHTV

SEQ ID NO: 7 - protein, 544 aa, Homo sapiens
MWLKVGGLLRGTGGQLGQTVGWPCGALGPGPHRWGPCGGSWAQKFYQDGPGRGLGEEDIRRAREARPRKTPRPQLSDRSRERKVPASRISRLANFGGLAVGLGLGVLAEMAKKSMPGGRLQSEGGSGLDSSPFLSEANAERIVQTLCTVRGAALKVGQMLSIQDNSFISPQLQHIFERVRQSADFMPRWQMLRVLEEELGRDWQAKVASLEEVPFAAASIGQVHQGLLRDGTEVAVKIQYPGIAQSIQSDVQNLLAVLKMSAALPAGLFAEQSLQALQQELAWECDYRREAACAQNFRQLLANDPFFRVPAVVKELCTTRVLGMELAGGVPLDQCQGLSQDLRNQICFQLLTLCLRELFEFRFMQTDPNWANFLYDASSHQVTLLDFGASREFGTEFTDHYIEVVKAAADGDRDCVLQKSRDLKFLTGFETKAFSDAHVEAVMILGEPFATQGPYDFGSGETARRIQDLIPVLLRHRLCPPPEETYALHRKLAGAFLACAHLRAHIACRDLFQDTYHRYWASRQPDAATAGSLPTKGDSWVDPS

SEQ ID NO: 8 - protein, 2017 aa, Homo sapiens
MSLGLAGAQEVELTLETVIQTLESSVLCQEKGLGARDLAQDAQITSLPALIREIVTRNLSQPESPVLLPATEMASLLSLQEENQLLQQELSRVEDLLAQSRAERDELAIKYNAVSERLEQALRLEPGELETQEPRGLVRQSVELRRQLQEEQASYRRKLQAYQEGQQRQAQLVQRLQGKILQYKKRCSELEQQLLERSGELEQQRLRDTEHSQDLESALIRLEEEQQRSASLAQVNAMLREQLDQAGSANQALSEDIRKVTNDWTRCRKELEHREAAWRREEESFNAYFSNEHSRLLLLWRQVVGFRRLVSEVKMFTERDLLQLGGELARTSRAVQEAGLGLSTGLRLAESRAEAALEKQALLQAQLEEQLRDKVLREKDLAQQQMQSDLDKADLSARVTELGLAVKRLEKQNLEKDQVNKDLTEKLEALESLRLQEQAALETEDGEGLQQTLRDLAQAVLSDSESGVQLSGSERTADASNGSLRGLSGQRTPSPPRRSSPGRGRSPRRGPSPACSDSSTLALIHSALHKRQLQVQDMRGRYEASQDLLGTLRKQLSDSESERRALEEQLQRLRDKTDGAMQAHEDAQREVQRLRSANELLSREKSNLAHSLQVAQQQAEELRQEREKLQAAQEELRRQRDRLEEEQEDAVQDGARVRRELERSHRQLEQLEGKRSVLAKELVEVREALSRATLQRDMLQAEKAEVAEALTKAEAGRVELELSMTKLRAEEASLQDSLSKLSALNESLAQDKLDLNRLVAQLEEEKSALQGRQRQAEQEATVAREEQERLEELRLEQEVARQGLEGSLRVAEQAQEALEQQLPTLRHERSQLQEQLAQLSRQLSGREQELEQARREAQRQVEALERAAREKEALAKEHAGLAVQLVAAEREGRTLSEEATRLRLEKEALEGSLFEVQRQLAQLEARREQLEAEGQALLLAKETLTGELAGLRQQIIATQEKASLDKELMAQKLVQAEREAQASLREQRAAHEEDLQRLQREKEAAWRELEAERAQLQSQLQREQEELLARLEAEKEELSEEIAALQQERDEGLLLAESEKQQALSLKESEKTALSEKLMGTRHSLATISLEMERQKRDAQSRQEQDRSTVNALTSELRDLRAQREEAAAAHAQEVRRLQEQARDLGKQRDSCLREAEELRTQLRLLEDARDGLRRELLEAQRKLRESQEGREVQRQEAGELRRSLGEGAKEREALRRSNEELRSAVKKAESERISLKLANEDKEQKLALLEEARTAVGKEAGELRTGLQEVERSRLEARRELQELRRQMKMLDSENTRLGRELAELQGRLALGERAEKESRRETLGLRQRLLKGEASLEVMRQELQVAQRKLQEQEGEFRTRERRLLGSLEEARGTEKQQLDHARGLELKLEAARAEAAELGLRLSAAEGRAQGLEAELARVEVQRRAAEAQLGGLRSALRRGLGLGRAPSPAPRPVPGSPARDAPAEGSGEGLNSPSTLECSPGSQPPSPGPATSPASPDLDPEAVRGALREFLQELRSAQRERDELRTQTSALNRQLAEMEAERDSATSRARQLQKAVAESEEARRSVDGRLSGVQAELALQEESVRRSERERRATLDQVATLERSLQATESELRASQEKISKMKANETKLEGDKRRLKEVLDASESRTVKLELQRRSLEGELQRSRLGLSDREAQAQALQDRVDSLQRQVADSEVKAGTLQLTVERLNGALAKVEESEGALRDKVRGLTEALAQSSASLNSTRDKNLHLQKALTACEHDRQVLQERLDAARQALSEARKQSSSLGEQVQTLRGEVADLELQRVEAEGQLQQLREVLRQRQEGEAAALNTVQKLQDERRLLQERLGSLQRALAQLEAEKREVERSALRLEKDRVALRRTLDKVEREKLRSHEDTVRLSAEKGRLDRTLTGAELELAEAQRQIQQLEAQVVVLEQSHSPAQLEVDAQQQQLELQQEVERLRSAQAQTERTLEARERAHRQRVRGLEEQVSTLKGQLQQELRRSSAPFSPPSGPPEK

SEQ ID NO: 9 - protein, 1505 aa, Homo sapiens
MLCVAGARLKRELDATATVLANRQDESEQSRKRLIEQSREFKKNTPEDLRKQVAPLLKSFQGEIDALSKRSKEAEAAFLNVYKRLIDVPDPVPALDLGQQLQLKVQRLHDIETENQKLRETLEEYNKEFAEVKNQEVTIKALKEKIREYEQTLKNQAETIALEKEQKLQNDFAEKERKLQETQMSTTSKLEEAEHKVQSLQTALEKTRTELFDLKTKYDEETTAKADEIEMIMTDLERANQRAEVAQREAETLREQLSSANHSLQLASQIQKAPDVEQAIEVLTRSSLEVELAAKEREIAQLVEDVQRLQASLTKLRENSASQISQLEQQLSAKNSTLKQLEEKLKGQADYEEVKKELNILKSMEFAPSEGAGTQDAAKPLEVLLLEKNRSLQSENAALRISNSDLSGSARRKGKDQPESRRPGSLPAPPPSQLPRNPGEQASNTNGTHQFSPAGLSQDFFSSSLASPSLPLASTGKFALNSLLQRQLMQSFYSKAMQEAGSTSMIFSTGPYSTNSISSQSPLQQSPDVNGMAPSPSQSESAGSVSEGEEMDTAEIARQVKEQLIKHNIGQRIFGHYVLGLSQGSVSEILARPKPWNKLTVRGKEPFHKMKQFLSDEQNILALRSIQGRQRENPGQSLNRLFQEVPKRRNGSEGNITTRIRASETGSDEAIKSILEQAKRELQVQKTAEPAQPSSASGSGNSDDAIRSILQQARREMEAQQAALDPALKQAPLSQSDITILTPKLLSTSPMPTVSSYPPLAISLKKPSAAPEAGASALPNPPALKKEAQDAPGLDPQGAADCAQGVLRQVKNEVGRSGAWKDHWWSAVQPERRNAASSEEAKAEETGGGKEKGSGGSGGGSQPRAERSQLQGPSSSEYWKEWPSAESPYSQSSELSLTGASRSETPQNSPLPSSPIVPMSKPTKPSVPPLTPEQYEVYMYQEVDTIELTRQVKEKLAKNGICQRIFGEKVLGLSQGSVSDMLSRPKPWSKLTQKGREPFIRMQLWLNGELGQGVLPVQGQQQGPVLHSVTSLQDPLQQGCVSSESTPKTSASCSPAPESPMSSSESVKSLTELVQQPCPPIEASKDSKPPEPSDPPASDSQPTTPLPLSGHSALSIQELVAMSPELDTYGITKRVKEVLTDNNLGQRLFGETILGLTQGSVSDLLARPKPWHKLSLKGREPFVRMQLWLNDPNNVEKLMDMKRMEKKAYMKRRHSSVSDSQPCEPPSVGTEYSQGASPQPQHQLKKPRVVLAPEEKEALKRAYQQKPYPSPKTIEDLATQLNLKTSTVINWFHNYRSRIRRELFIEEIQAGSQGQAGASDSPSARSGRAAPSSEGDSCDGVEATEGPGSADTEEPKSQGEAEREEVPRPAEQTEPPPSGTPGPDDARDDDHEGGPVEGPGPLPSPASATATAAPAAPEDAATSAAAAPGEGPAAPSSAPPPSNSSSSSAPRRPSSLQSLFGLPEAAGARDSRDNPLRKKKAANLNSIIHRLEKAASREEPIEWEF

SEQ ID NO: 10 - protein, 350 aa, Homo sapiens
MQRLGATLLCLLLAAAVPTAPAPAPTATSAPVKPGPALSYPQEEATLNEMFREVEELMEDTQHKLRSAVEEMEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALREPAAAAAALLGGEEI

SEQ ID NO: 11 - protein, 328 aa, Homo sapiens
MAAGIVASRRLRDLLTRRLTGSNYPGLSISLRLTGSSAQEEASGVALGEAPDHSYESLRVTSAQKHVLHVQLNRPNKRNAMNKVFWREMVECFNKISRDADCRAVVISGAGKMFTAGIDLMDMASDILQPKGDDVARISWYLRDIITRYQETFNVIERCPKPVIAAVHGGCIGGGVDLVTACDIRYCAQDAFFQVKEVDVGLAADVGTLQRLPKVIGNQSLVNELAFTARKMMADEALGSGLVSRVFPDKEVMLDAALALAAEISSKSPVAVQSTKVNLLYSRDHSVAESLNYVASWNMSMLQTQDLVKSVQATTENKELKTVTFSKL

SEQ ID NO: 12 - protein, 998 aa, Homo sapiens
MARARPPPPPSPPPGLLPLLPPLLLLPLLLLPAGCRALEETLMDTKWVTSELAWTSHPESGWEEVSGYDEAMNPIRTYQVCNVRESSQNNWLRTGFIWRRDVQRVYVELKFTVRDCNSIPNIPGSCKETFNLFYYEADSDVASASSPFWMENPYVKVDTIAPDESFSRLDAGRVNTKVRSFGPLSKAGFYLAFQDQGACMSLISVRAFYKKCASTTAGFALFPETLTGAEPTSLVIAPGTCIPNAVEVSVPLKLYCNGDGEWMVPVGACTCATGHEPAAKESQCRPCPPGSYKAKQGEGPCLPCPPNSRTTSPAASICTCHNNFYRADSDSADSACTTVPSPPRGVISNVNETSLILEWSEPRDLGGRDDLLYNVICKKCHGAGGASACSRCDDNVEFVPRQLGLTERRVHISHLLAHTRYTFEVQAVNGVSGKSPLPPRYAAVNITTNQAAPSEVPTLRLHSSSGSSLTLSWAPPERPNGVILDYEMKYFEKSEGIASTVTSQMNSVQLDGLRPDARYVVQVRARTVAGYGQYSRPAEFETTSERGSGAQQLQEQLPLIVGSATAGLVFVVAVVVIAIVCLRKQRHGSDSEYTEKLQQYIAPGMKVYIDPFTYEDPNEAVREFAKEIDVSCVKIEEVIGAGEFGEVCRGRLKQPGRREVFVAIKTLKVGYTERQRRDFLSEASIMGQFDHPNIIRLEGVVTKSRPVMILTEFMENCALDSFLRLNDGQFTVIQLVGMLRGIAAGMKYLSEMNYVHRDLAARNILVNSNLVCKVSDFGLSRFLEDDPSDPTYTSSLGGKIPIRWTAPEAIAYRKFTSASDVWSYGIVMWEVMSYGERPYWDMSNQDVINAVEQDYRLPPPMDCPTALHQLMLDCWVRDRNLRPKFSQIVNTLDKLIRNAASLKVIASAQSGMSQPLLDRTVPDYTTFTTVGDWLDAIKMGRYKESFVSAGFASFDLVAQMTAEDLLRIGVTLAGHQKKILSSIQDMRLQMNQTLPVQV

SEQ ID NO: 13 - protein, 889 aa, Homo sapiens
MSYFGEHFWGEKNHGFEVLYHSVKQGPISTKELADFIRERATIEETYSKAMAKLSKLASNGTPMGTFAPLWEVFRVSSDKLALCHLELTRKLQDLIKDVLRYGEEQLKTHKKCKEEVVSTLDAVQVLSGVSQLLPKSRENYLNRCMDQERLRRESTSQKEMDKAETKTKKAAESLRRSVEKYNSARADFEQKMLDSALRFQAMEETHLRHMKALLGSYAHSVEDTHVQIGQVHEEFKQNIENVSVEMLLRKFAESKGTGREKPGPLDFEAYSAAALQEAMKRLRGAKAFRLPGLSRREREPEPPAAVDFLEPDSGTCPEVDEEGFTVRPDVTQNSTAEPSRFSSSDSDFDDEEPRKFYVHIKPAPARAPACSPEAAAAQLRATAGSLILPPGPGGTMKRHSSRDAAGKPQRPRSAPRTSSCAERLQSEEQVSKNLFGPPLESAFDHEDFTGSSSLGFTSSPSPFSSSSPENVEDSGLDSPSHAAPGPSPDSWVPRPGTPQSPPSCRAPPPEARGIRAPPLPDSPQPLASSPGPWGLEALAGGDLMPAPADPTAREGLAAPPRRLRSRKVSCPLTRSNGDLSRSLSPSPLGSSAASTALERPSFLSQTGHGVSRGPSPVVLGSQDALPIATAFTEYVHAYFRGHSPSCLARVTGELTMTFPAGIVRVFSGTPPPPVLSFRLVHTTAIEHFQPNADLLFSDPSQSDPETKDFWLNMAALTEALQRQAEQNPTASYYNVVLLRYQFSRPGPQSVPLQLSAHWQCGATLTQVSVEYGYRPGATAVPTPLTNVQILLPVGEPVTNVRLQPAATWNLEEKRLTWRLPDVSEAGGSGRLSASWEPLSGPSTPSPVAAQFTSEGTTLSGVDLELVGSGYRMSLVKRRFATGMYLVSC

SEQ ID NO: 14 - protein, 461 aa, Homo sapiens
MVNDRWKTMGGAAQLEDRPRDKPQRPSCGYVLCTVLLALAVLLAVAVTGAVLFLNHAHAPGTAPPPVVSTGAASANSALVTVERADSSHLSILIDPRCPDLTDSFARLESAQASVLQALTEHQAQPRLVGDQEQELLDTLADQLPRLLARASELQTECMGLRKGHGTLGQGLSALQSEQGRLIQLLSESQGHMAHLVNSVSDILDALQRDRGLGRPRNKADLQRAPARGTRPRGCATGSRPRDCLDVLLSGQQDDGVYSVFPTHYPAGFQVYCDMRTDGGGWTVFQRREDGSVNFFRGWDAYRDGFGRLTGEHWLGLKRIHALTTQAAYELHVDLEDFENGTAYARYGSFGVGLFSVDPEEDGYPLTVADYSGTAGDSLLKHSGMRFTTKDRDSDHSENNCAAFYRGAWWYRNCHTSNLNGQYLRGAHASYADGVEWSSWTGWQYSLKFSEMKIRPVREDR

SEQ ID NO: 15 - protein, 605 aa, Homo sapiens
MVRHQPLQYYEPQLCLSCLTGIYGCRWKRYQRSHDDTTPWERLWFLLLTFTFGLTLTWLYFWWEVHNDYDEFNWYLYNRMGYWSDWPVPILVTTAAAFAYIAGLLVLALCHIAVGQQMNLHWLHKIGLVVILASTVVAMSAVAQLWEDEWEVLLISLQGTAPFLHVGAVAAVTMLSWIVAGQFARAERTSSQVTILCTFFTVVFALYLAPLTISSPCIMEKKDLGPKPALIGHRGAPMLAPEHTLMSFRKALEQKLYGLQADITISLDGVPFLMHDTTLRRTTNVEEEFPELARRPASMLNWTTLQRLNAGQWFLKTDPFWTASSLSPSDHREAQNQSICSLAELLELAKGNATLLLNLRDPPREHPYRSSFINVTLEAVLHSGFPQHQVMWLPSRQRPLVRKVAPGFQQTSGSKEAVASLRRGHIQRLNLRYTQVSRQELRDYASWNLSVNLYTVNAPWLFSLLWCAGVPSVTSDNSHALSQVPSPLWIMPPDEYCLMWVTADLVSFTLIVGIFVLQKWRLGGIRSYNPEQIMLSAAVRRTSRDVSIMKEKLIFSEISDGVEVSDVLSVCSDNSYDTYANSTATPVGPRGGGSHTKTLIERSGR

SEQ ID NO: 16 - protein, 469 aa, Homo sapiens
MSIHFSSPVFTSRSAAFSGRGAQVRLSSARPGGLGSSSLYGLGASRPRVAVRSAYGGPVGAGIREVTINQSLLAPLRLDADPSLQRVRQEESEQIKTLNNKFASFIDKVRFLEQQNKLLETKWTLLQEQKSAKSSRLPDIFEAQIAGLRGQLEALQVDGGRLEAELRSMQDVVEDFKNKYEDEINHRTAAENEFVVLKKDVDAAYMSKVELEAKVDALNDEINFLRTLNETELTELQSQISDTSVVLSMDNSRSLDLDGIIAEVKAQYEEMAKCSRAEAEAWYQTKFETLQAQAGKHGDDLRNTRNEISEMNRAIQRLQAEIDNIKNQRAKLEAAIAEAEERGELALKDARAKQEELEAALQRGKQDMARQLREYQELMSVKLALDIEIATYRKLLEGEESRLAGDGVGAVNISVMNSTGGSSSGGGIGLTLGGTMGSNALSFSSSAGPGLLKAYSIRTASASRRSARD

SEQ ID NO: 17 - protein, 638 aa, Homo sapiens
MSALAGEDVWRCPGCGDHIAPSQIWYRTVNETWHGSCFRCSECQDSLTNWYYEKDGKLYCPKDYWGKFGEFCHGCSLLMTGPFMVAGEFKYHPECFACMSCKVIIEDGDAYALVQHATLYCGKCHNEVVLAPMFERLSTESVQEQLPYSVTLISMPATTEGRRGFSVSVESACSNYATTVQVKEVNRMHISPNNRNAIHPGDRILEINGTPVRTLRVEEVEDAISQTSQTLQLLIEHDPVSQRLDQLRLEARLAPHMQNAGHPHALSTLDTKENLEGTLRRRSLRRSNSISKSPGPSSPKEPLLFSRDISRSESLRCSSSYSQQIFRPCDLIHGEVLGKGFFGQAIKVTHKATGKVMVMKELIRCDEETQKTFLTEVKVMRSLDHPNVLKFIGVLYKDKKLNLLTEYIEGGTLKDFLRSMDPFPWQQKVRFAKGIASGMAYLHSMCIIHRDLNSHNCLIKLDKTVVVADFGLSRLIVEERKRAPMEKATTKKRTLRKNDRKKRYTVVGNPYWMAPEMLNGKSYDETVDIFSFGIVLCEIIGQVYADPDCLPRTLDFGLNVKLFWEKFVPTDCPPAFFPLAAICCRLEPESRPAFSKLEDSFEALSLYLGELGIPLPAELEELDHTVSMQYGLTRDSPP

SEQ ID NO: 18 - protein, 1460 aa, Homo sapiens
MPAPGALILLAAVSASGCLASPAHPDGFALGRAPLAPPYAVVLISCSGLLAFIFLLLTCLCCKRGDVGFKEFENPEGEDCSGEYTPPAEETSSSQSLPDVYILPLAEVSLPMPAPQPSHSDMTTPLGLSRQHLSYLQEIGSGWFGKVILGEIFSDYTPAQVVVKELRASAGPLEQRKFISEAQPYRSLQHPNVLQCLGLCVETLPFLLIMEFCQLGDLKRYLRAQRPPEGLSPELPPRDLRTLQRMGLEIARGLAHLHSHNYVHSDLALRNCLLTSDLTVRIGDYGLAHSNYKEDYYLTPERLWIPLRWAAPELLGELHGTFMVVDQSRESNIWSLGVTLWELFEFGAQPYRHLSDEEVLAFVVRQQHVKLARPRLKLPYADYWYDILQSCWRPPAQRPSASDLQLQLTYLLSERPPRPPPPPPPPRDGPFPWPWPPAHSAPRPGTLSSPFPLLDGFPGADPDDVLTVTESSRGLNLECLWEKARRGAGRGGGAPAWQPASAPPAPHANPSNPFYEALSTPSVLPVISARSPSVSSEYYIRLEEHGSPPEPLFPNDWDPLDPGVPAPQAPQAPSEVPQLVSETWASPLFPAPRPFPAQSSASGSFLLSGWDPEGRGAGETLAGDPAEVLGERGTAPWVEEEEEEEEGSSPGEDSSSLGGGPSRRGPLPCPLCSREGACSCLPLERGDAVAGWGGHPALGCPHPPEDDSSLRAERGSLADLPMAPPASAPPEFLDPLMGAAAPQYPGRGPPPAPPPPPPPPRAPADPAASPDPPSAVASPGSGLSSPGPKPGDSGYETETPFSPEGAFPGGGAAEEEGVPRPRAPPEPPDPGAPRPPPDPGPLPLPGPREKPTFVVQVSTEQLLMSLREDVTRNLLGEKGATARETGPRKAGRGPGNREKVPGLNRDPTVLGNGKQAPSLSLPVNGVTVLENGDQRAPGIEEKAAENGALGSPEREEKVLENGELTPPRREEKALENGELRSPEAGEKVLVNGGLTPPKSEDKVSENGGLRFPRNTERPPETGPWRAPGPWEKTPESWGPAPTIGEPAPETSLERAPAPSAVVSSRNGGETAPGPLGPAPKNGTLEPGTERRAPETGGAPRAPGAGRLDLGSGGRAPVGTGTAPGGGPGSGVDAKAGWVDNTRPQPPPPPLPPPPEAQPRRLEPAPPRARPEVAPEGEPGAPDSRAGGDTALSGDGDPPKPERKGPEMPRLFLDLGPPQGNSEQIKARLSRLSLALPPLTLTPFPGPGPRRPPWEGADAGAAGGEAGGAGAPGPAEEDGEDEDEDEEEDEEAAAPGAAAGPRGPGRARAAPVPVVVSSADADAARPLRGLLKSPRGADEPEDSELERKRKMVSFHGDVTVYLFDQETPTNELSVQAPPEGDTDPSTPPAPPTPPHPATPGDGFPSNDSGFGGSFEWAEDFPLLPPPGPPLCFSRFSVSPALETPGPPARAPDARPAGPVEN

SEQ ID NO: 19 - protein, 574 aa, Homo sapiens
MALARGSRQLGALVWGACLCVLVHGQQAQPGQGSDPARWRQLIQWENNGQVYSLLNSGSEYVPAGPQRSESSSRVLLAGAPQAQQRRSHGSPRRRQAPSLPLPGRVGSDTVRGQARHPFGFGQVPDNWREVAVGDSTGMARARTSVSQQRHGGSASSVSASAFASTYRQQPSYPQQFPYPQAPFVSQYENYDPASRTYDQGFVYYRPAGGGVGAGAAAVASAGVIYPYQPRARYEEYGGGEELPEYPPQGFYPAPERPYVPPPPPPPDGLDRRYSHSLYSEGTPGFEQAYPDPGPEAAQAHGGDPRLGWYPPYANPPPEAYGPPRALEPPYLPVRSSDTPPPGGERNGAQQGRLSVGSVYRPNQNGRGLPDLVPDPNYVQASTYVQRAHLYSLRCAAEEKCLASTAYAPEATDYDVRVLLRFPQRVKNQGTADFLPNRPRHTWEWHSCHQHYHSMDEFSHYDLLDAATGKKVAEGHKASFCLEDSTCDFGNLKRYACTSHTQGLSPGCYDTYNADIDCQWIDITDVQPGNYILKVHVNPKYIVLESDFTNNVVRCNIHYTGRYVSATNCKIVQS

SEQ ID NO: 20 - protein, 1136 aa, Homo sapiens
MAKMEVKTSLLDNMIGVGDMVLLEPLNEETFINNLKKRFDHSEIYTYIGSVVISVNPYRSLPIYSPEKVEEYRNRNFYELSPHIFALSDEAYRSLRDQDKDQCILITGESGAGKTEASKLVMSYVAAVCGKGAEVNQVKEQLLQSNPVLEAFGNAKTVRNDNSSRFGKYMDIEFDFKGDPLGGVISNYLLEKSRVVKQPRGERNFHVFYQLLSGASEELLNKLKLERDFSRYNYLSLDSAKVNGVDDAANFRTVRNAMQIVGFMDHEAESVLAVVAAVLKLGNIEFKPESRVNGLDESKIKDKNELKEICELTGIDQSVLERAFSFRTVEAKQEKVSTTLNVAQAYYARDALAKNLYSRLFSWLVNRINESIKAQTKVRKKVMGVLDIYGFEIFEDNSFEQFIINYCNEKLQQIFIELTLKEEQEEYIREDIEWTHIDYFNNAIICDLIENNTNGILAMLDEECLRPGTVTDETFLEKLNQVCATHQHFESRMSKCSRFLNDTSLPHSCFRIQHYAGKVLYQVEGFVDKNNDLLYRDLSQAMWKASHALIKSLFPEGNPAKINLKRPPTAGSQFKASVATLMKNLQTKNPNYIRCIKPNDKKAAHIFNEALVCHQIRYLGLLENVRVRRAGYAFRQAYEPCLERYKMLCKQTWPHWKGPARSGVEVLFNELEIPVEEYSFGRSKIFIRNPRTLFKLEDLRKQRLEDLATLIQKIYRGWKCRTHFLLMKKSQIVIAAWYRRYAQQKRYQQTKSSALVIQSYIRGWKARKILRELKHQKRCKEAVTTIAAYWHGTQARRELRRLKEEARNKHAIAVIWAYWLGSKARRELKRLKEEARRKHAVAVIWAYWLGLKVRREYRKFFRANAGKKIYEFTLQRIVQKYFLEMKNKMPSLSPIDKNWPSRPYLFLDSTHKELKRIFHLWRCKKYRDQFTDQQKLIYEEKLEASELFKDKKALYPSSVGQPFQGAYLEINKNPKYKKLKDAIEEKIIIAEVVNKINRANGKSTSRIFLLTNNNLLLADQKSGQIKSEVPLVDVTKVSMSSQNDGFFAVHLKEGSEAASKGDFLFSSDHLIEMATKLYRTTLSQTKQKLNIEISDEFLVQFRQDKVCVKFIQGNQKNGSVPTCKRKNNRLLEVAVP

SEQ ID NO: 21 - protein, 1060 aa, Homo sapiens
MDSGAGGRRCPEAALLILGPPRMEHLRHSPGPGGQRLLLPSMLLALLLLLAPSPGHATRVVYKVPEEQPPNTLIGSLAADYGFPDVGHLYKLEVGAPYLRVDGKTGDIFTTETSIDREGLRECQNQLPGDPCILEFEVSITDLVQNGSPRLLEGQIEVQDINDNTPNFASPVITLAIPENTNIGSLFPIPLASDRDAGPNGVASYELQAGPEAQELFGLQVAEDQEEKQPQLIVMGNLDRERWDSYDLTIKVQDGGSPPRASSALLRVTVLDTNDNAPKFERPSYEAELSENSPIGHSVIQVKANDSDQGANAEIEYTFHQAPEVVRRLLRLDRNTGLITVQGPVDREDLSTLRFSVLAKDRGTNPKSARAQVVVTVKDMNDNAPTIEIRGIGLVTHQDGMANISEDVAEETAVALVQVSDRDEGENAAVTCVVAGDVPFQLRQASETGSDSKKKYFLQTTTPLDYEKVKDYTIEIVAVDSGNPPLSSTNSLKVQVVDVNDNAPVFTQSVTEVAFPENNKPGEVIAEITASDADSGSNAELVYSLEPEPAAKGLFTISPETGEIQVKTSLDREQRESYELKVVAADRGSPSLQGTATVLVNVLDCNDNDPKFMLSGYNFSVMENMPALSPVGMVTVIDGDKGENAQVQLSVEQDNGDFVIQNGTGTILSSLSFDREQQSTYTFQLKAVDGGVPPRSAYVGVTINVLDENDNAPYITAPSNTSHKLLTPQTRLGETVSQVAAEDFDSGVNAELIYSIAGGNPYGLFQIGSHSGAITLEKEIERRHHGLHRLVVKVSDRGKPPRYGTALVHLYVNETLANRTLLETLLGHSLDTPLDIDIAGDPEYERSKQRGNILFGVVAGVVAVALLIALAVLVRYCRQREAKSGYQAGKKETKDLYAPKPSGKASKGNKSKGKKSKSPKPVKPVEDEDEAGLQKSLKFNLMSDAPGDSPRIHLPLNYPPGSPDLGRHYRSNSPLPSIQLQPQSPSASKKHQVVQDLPPANTFVGTGDTTSTGSEQYSDYSYRTNPPKYPSKQVGQPFQLSTPQPLPHPYHGAIWTEVWE

SEQ ID NO: 22 - protein, 329 aa, Homo sapiens
MTTQQIDLQGPGPWGFRLVGGKDFEQPLAISRVTPGSKAALANLCIGDVITAIDGENTSNMTHLEAQNRIKGCTDNLTLTVARSEHKVWSPLVTEEGKRHPYKMNLASEPQEVLHIGSAHNRSAMPFTASPASSTTARVITNQYNNPAGLYSSENISNFNNALESKTAASGVEANSRPLDHAQPPSSLVIDKESEVYKMLQEKQELNEPPKQSTSFLVLQEILESEEKGDPNKPSGFRSVKAPVTKVAASIGNAQKLPMCDKCGTGIVGVFVKLRDRHRHPECYVCTDCGTNLKQKGHFFVEDQIYCEKHARERVTPPEGYEVVTVFPK

SEQ ID NO: 23 - protein, 806 aa, Homo sapiens
MQFFGRLVNTFSGVTNLFSNPFRVKEVAVADYTSSDRVREEGQLILFQNTPNRTWDCVLVNPRNSQSGFRLFQLELEADALVNFHQYSSQLLPFYESSPQVLHTEVLQHLTDLIRNHPSWSVAHLAVELGIRECFHHSRIISCANCAENEEGCTPLHLACRKGDGEILVELVQYCHTQMDVTDYKGETVFHYAVQGDNSQVLQLLGRNAVAGLNQVNNQGLTPLHLACQLGKQEMVRVLLLCNARCNIMGPNGYPIHSAMKFSQKGCAEMIISMDSSQIHSKDPRYGASPLHWAKNAEMARMLLKRGCNVNSTSSAGNTALHVAVMRNRFDCAIVLLTHGANADARGEHGNTPLHLAMSKDNVEMIKALIVFGAEVDTPNDFGETPTFLASKIGRLVTRKAILTLLRTVGAEYCFPPIHGVPAEQGSAAPHHPFSLERAQPPPISLNNLELQDLMHISRARKPAFILGSMRDEKRTHDHLLCLDGGGVKGLIIIQLLIAIEKASGVATKDLFDWVAGTSTGGILALAILHSKSMAYMRGMYFRMKDEVFRGSRPYESGPLEEFLKREFGEHTKMTDVRKPKVMLTGTLSDRQPAELHLFRNYDAPETVREPRFNQNVNLRPPAQPSDQLVWRAARSSGAAPTYFRPNGRFLDGGLLANNPTLDAMTEIHEYNQDLIRKGQANKVKKLSIVVSLGTGRSPQVPVTCVDVFRPSNPWELAKTVFGAKELGKMVVDCCTDPDGRAVDRARAWCEMVGIQYFRLNPQLGTDIMLDEVSDTVLVNALWETEVYIYEHREEFQKLIQLLLSP

SEQ ID NO: 24 - protein, 567 aa, Homo sapiens
MHGRLKVKTSEEQAEAKRLEREQKLKLYQSATQAVFQKRQAGELDESVLELTSQILGANPDFATLWNCRREVLQQLETQKSPEELAALVKAELGFLESCLRVNPKSYGTWHHRCWLLGRLPEPNWTRELELCARFLEVDERNFHCWDYRRFVATQAAVPPAEELAFTDSLITRNFSNYSSWHYRSCLLPQLHPQPDSGPQGRLPEDVLLKELELVQNAFFTDPNDQSAWFYHRWLLGRADPQDALRCLHVSRDEACLTVSFSRPLLVGSRMEILLLMVDDSPLIVEWRTPDGRNRPSHVWLCDLPAASLNDQLPQHTFRVIWTAGDVQKECVLLKGRQEGWCRDSTTDEQLFRCELSVEKSTVLQSELESCKELQELEPENKWCLLTIILLMRALDPLLYEKETLQYFQTLKAVDPMRATYLDDLRSKFLLENSVLKMEYAEVRVLHLAHKDLTVLCHLEQLLLVTHLDLSHNRLRTLPPALAALRCLEVLQASDNAIESLDGVTNLPRLQELLLCNNRLQQPAVLQPLASCPRLVLLNLQGNPLCQAVGILEQLAELLPSVSSVLT

SEQ ID NO: 25 - protein, 669 aa, Homo sapiens
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 26 - protein, 707 aa, Homo sapiens
MAAAATAAEGVPSRGPPGEVIHLNVGGKRFSTSRQTLTWIPDSFFSSLLSGRISTLKDETGAIFIDRDPTVFAPILNFLRTKELDPRGVHGSSLLHEAQFYGLTPLVRRLQLREELDRSSCGNVLFNGYLPPPVFPVKRRNRHSLVGPQQLGGRPAPVRRSNTMPPNLGNAGLLGRMLDEKTPPSPSGQPEEPGMVRLVCGHHNWIAVAYTQFLVCYRLKEASGWQLVFSSPRLDWPIERLALTARVHGGALGEHDKMVAAATGSEILLWALQAEGGGSEIGVFHLGVPVEALFFVGNQLIATSHTGRIGVWNAVTKHWQVQEVQPITSYDAAGSFLLLGCNNGSIYYVDVQKFPLRMKDNDLLVSELYRDPAEDGVTALSVYLTPKTSDSGNWIEIAYGTSSGGVRVIVQHPETVGSGPQLFQTFTVHRSPVTKIMLSEKHLISVCADNNHVRTWSVTRFRGMISTQPGSTPLASFKILALESADGHGGCSAGNDIGPYGERDDQQVFIQKVVPSASQLFVRLSSTGQRVCSVRSVDGSPTTAFTVLECEGSRRLGSRPRRYLLTGQANGSLAMWDLTTAMDGLGQAPAGGLTEQELMEQLEHCELAPPAPSAPSWGCLPSPSPRISLTSLHSASSNTSLSGHRGSPSPPQAEARRRGGGSFVERCQELVRSGPDLRRPPTPAPWPSSGLGTPLTPPKMKLNETSF

SEQ ID NO: 27 - protein, 475 aa, Homo sapiens
MAVVSEDDFQHSSNSTYRTTSSSLRADQEALLEKLLDRPPPGLQRPEDRFCGTYIIFFSLGIGSLLPWNFFITAKEYWMFKLRNSSSPATGEDPEGSDILNYFESYLAVASTVPSMLCLVANFLLVNRVAVHIRVLASLTVILAIFMVITALVKVDTSSWTRGFFAVTIVCMVILSGASTVFSSSIYGMTGSFPMRNSQALISGGAMGGTVSAVASLVDLAASSDVRNSALAFFLTATVFLVLCMGLYLLLSRLEYARYYMRPVLAAHVFSGEEELPQDSLSAPSVASRFIDSHTPPLRPILKKTASLGFCVTYVFFITSLIYPAICTNIESLNKGSGSLWTTKFFIPLTTFLLYNFADLCGRQLTAWIQVPGPNSKALPGFVLLRTCLIPLFVLCNYQPRVHLKTVVFQSDVYPALLSSLLGLSNGYLSTLALLYGPKIVPRELAEATGVVMSFYVCLGLTLGSACSTLLVHLI

SEQ ID NO: 28 - protein, 654 aa, Homo sapiens
MNGHLEAEEQQDQRPDQELTGSWGHGPRSTLVRAKAMAPPPPPLAASTPLLHGEFGSYPARGPRFALTLTSQALHIQRLRPKPEARPRGGLVPLAEVSGCCTLRSRSPSDSAAYFCIYTYPRGRRGARRRATRTFRADGAATYEENRAEAQRWATALTCLLRGLPLPGDGEITPDLLPRPPRLLLLVNPFGGRGLAWQWCKNHVLPMISEAGLSFNLIQTERQNHARELVQGLSLSEWDGIVTVSGDGLLHEVLNGLLDRPDWEEAVKMPVGILPCGSGNALAGAVNQHGGFEPALGLDLLLNCSLLLCRGGGHPLDLLSVTLASGSRCFSFLSVAWGFVSDVDIQSERFRALGSARFTLGTVLGLATLHTYRGRLSYLPATVEPASPTPAHSLPRAKSELTLTPDPAPPMAHSPLHRSVSDLPLPLPQPALASPGSPEPLPILSLNGGGPELAGDWGGAGDAPLSPDPLLSSPPGSPKAALHSPVSEGAPVIPPSSGLPLPTPDARVGASTCGPPDHLLPPLGTPLPPDWVTLEGDFVLMLAISPSHLGADLVAAPHARFDDGLVHLCWVRSGISRAALLRLFLAMERGSHFSLGCPQLGYAAARAFRLEPLTPRGVLTVDGEQVEYGPLQAQMHPGIGTLLTGPPGCPGREP

SEQ ID NO: 29 - protein, 572 aa, Homo sapiens
MTSRDQPRPKGPPKSTSPCPGISNSESSPTLNYQGILNRLKQFPRFSPHFAAELESIYYSLHKIQQDVAEHHKQIGNVLQIVESCSQLQGFQSEEVSPAEPASPGTPQQVKDKTLQESSFEDIMATRSSDWLRRPLGEDNQPETQLFWDKEPWFWHDTLTEQLWRIFAGVHDEKAKPRDRQQAPGLGQESKAPGSCDPGTDPCPEDASTPRPPEASSSPPEGSQDRNTSWGVVQEPPGRASRFLQSISWDPEDFEDAWKRPDALPGQSKRLAVPCKLEKMRILAHGELVLATAISSFTRHVFTCGRRGIKVWSLTGQVAEDRFPESHLPIQTPGAFLRTCLLSSNSRSLLTGGYNLASVSVWDLAAPSLHVKEQLPCAGLNCQALDANLDANLAFASFTSGVVRIWDLRDQSVVRDLKGYPDGVKSIVVKGYNIWTGGPDACLRCWDQRTIMKPLEYQFKSQIMSLSHSPQEDWVLLGMANGQQWLQSTSGSQRHMVGQKDSVILSVKFSPFGQWWASVGMDDFLGVYSMPAGTKVFEVPEMSPVTCCDVSSNNRLVVTGSGEHASVYQITY

SEQ ID NO: 30 - protein, 277 aa, Homo sapiens
MASAELDYTIEIPDQPCWSQKNSPSPGGKEAETRQPVVILLGWGGCKDKNLAKYSAIYHKRGCIVIRYTAPWHMVFFSESLGIPSLRVLAQKLLELLFDYEIEKEPLLFHVFSNGGVMLYRYVLELLQTRRFCRLRVVGTIFDSAPGDSNLVGALRALAAILERRAAMLRLLLLVAFALVVVLFHVLLAPITALFHTHFYDRLQDAGSRWPELYLYSRADEVVLARDIERMVEARLARRVLARSVDFVSSAHVSHLRDYPTYYTSLCVDFMRNCVRC

SEQ ID NO: 31 - protein, 172 aa, Homo sapiens
MDSPSLRELQQPLLEGTECETPAQKPGRHELGSPLREIAFAESLRGLQFLSPPLPSVSAGLGEPRPPDVEDMSSSDSDSDWDGGSRLSPFLPHDHLGLAVFSMLCCFWPVGIAAFCLAQKTNKAWAKGDIQGAGAASRRAFLLGVLAVGLGVCTYAAALVTLAAYLASRDPP

SEQ ID NO: 32 - protein, 482 aa, Homo sapiens
MAQTQGTRRKVCYYYDGDVGNYYYGQGHPMKPHRIRMTHNLLLNYGLYRKMEIYRPHKANAEEMTKYHSDDYIKFLRSIRPDNMSEYSKQMQRFNVGEDCPVFDGLFEFCQLSTGGSVASAVKLNKQQTDIAVNWAGGLHHAKKSEASGFCYVNDIVLAILELLKYHQRVLYIDIDIHHGDGVEEAFYTTDRVMTVSFHKYGEYFPGTGDLRDIGAGKGKYYAVNYPLRDGIDDESYEAIFKPVMSKVMEMFQPSAVVLQCGSDSLSGDRLGCFNLTIKGHAKCVEFVKSFNLPMLMLGGGGYTIRNVARCWTYETAVALDTEIPNELPYNDYFEYFGPDFKLHISPSNMTNQNTNEYLEKIKQRLFENLRMLPHAPGVQMQAIPEDAIPEESGDEDEDDPDKRISICSSDKRIACEEEFSDSEEEGEGGRKNSSNFKKAKRVKTEDEKEKDPEEKKEVTEEEKTKEEKPEAKGVKEEVKLA

SEQ ID NO: 33 - DNA, 2118 bp, Homo sapiens
  source 1..2118: mol_type = other DNA
cccctcccccctgggtcggacgctgagcggagccgcgggcgggagggcggacggaccgactgacggtagggacgggaggcgagcaagatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctgaatggacctctccagctctggcttcctgctgagtccctcacgtttcttccccaacccctcagattttatattttctatttctctgtgtatttatataaaaatttattaaatataaatatccccagggacagaaaccaaggccccgagctcagggcagctgtgctgggtgagctcttccaggagccaccttgccacccattcttcccgttcttaactttgaaccataaagggtgccaggtctgggtgaaagggatacttttatgcaaccataagacaaactcctgaaatgccaagtgcctgcttagtagctttggaaaggtgcccttattgaacattctagaaggggtggctgggtcttcaaggatctcctgtttttttcaggctcctaaagtaacatcagccatttttagattggttctgttttcgtaccttcccactggcctcaagtgagccaagaaacactgcctgccctctgtctgtcttctcctaattctgcaggtggaggttgctagtctagtttcctttttgagatactattttcatttttgtgagcctctttgtaataaaatggtacatttctata

SEQ ID NO: 34 - protein, 952 aa, Homo sapiens
MDLRVGQRPPVEPPPEPTLLALQRPQRLHHHLFLAGLQQQRSVEPMRLSMDTPMPELQVGPQEQELRQLLHKDKSKRSAVASSVVKQKLAEVILKKQQAALERTVHPNSPGIPYRTLEPLETEGATRSMLSSFLPPVPSLPSDPPEHFPLRKTVSEPNLKLRYKPKKSLERRKNPLLRKESAPPSLRRRPAETLGDSSPSSSSTPASGCSSPNDSEHGPNPILGSEALLGQRLRLQETSVAPFALPTVSLLPAITLGLPAPARADSDRRTHPTLGPRGPILGSPHTPLFLPHGLEPEAGGTLPSRLQPILLLDPSGSHAPLLTVPGLGPLPFHFAQSLMTTERLSGSGLHWPLSRTRSEPLPPSATAPPPPGPMQPRLEQLKTHVQVIKRSAKPSEKPRLRQIPSAEDLETDGGGPGQVVDDGLEHRELGHGQPEARGPAPLQQHPQVLLWEQQRLAGRLPRGSTGDTVLLPLAQGGHRPLSRAQSSPAAPASLSAPEPASQARVLSSSETPARTLPFTTGLIYDSVMLKHQCSCGDNSRHPEHAGRIQSIWSRLQERGLRSQCECLRGRKASLEELQSVHSERHVLLYGTNPLSRLKLDNGKLAGLLAQRMFVMLPCGGVGVDTDTIWNELHSSNAARWAAGSVTDLAFKVASRELKNGFAVVRPPGHHADHSTAMGFCFFNSVAIACRQLQQQSKASKILIVDWDVHHGNGTQQTFYQDPSVLYISLHRHDDGNFFPGSGAVDEVGAGSGEGFNVNVAWAGGLDPPMGDPEYLAAFRIVVMPIAREFSPDLVLVSAGFDAAEGHPAPLGGYHVSAKCFGYMTQQLMNLAGGAVVLALEGGHDLTAICDASEACVAALLGNRVDPLSEEGWKQKPNLNAIRSLEAVIRVHSKYWGCMQRLASCPDSWVPRVPGADKEEVEAVTALASLSVGILAEDRPSEQLVEEEEPMNL

SEQ ID NO: 35 - DNA, 4102 bp, Homo sapiens
  source 1..4102: mol_type = other DNA
cttcctctcgccgtctcacagtcgctctgcagcctccggcgactggggggatgtgaggccggcgccccagccccccgccccgccatgagccccccgctctgagggccccggcccctggatgcacagccccggcgctgatgggacccaggtgagcccgggtgcccactactgcagccccactggcgcaggctgccccaggccctgtgcagacacaccaggccctcagccgcagcccatggacctgcgggtgggccagcggcccccagtggagcccccaccagagcccacattgctggccctgcagcgtccccagcgcctgcaccaccacctcttcctagcaggcctgcagcagcagcgctcggtggagcccatgaggctctccatggacacgccgatgcccgagttgcaggtgggaccccaggaacaagagctgcggcagcttctccacaaggacaagagcaagcgaagtgctgtagccagcagcgtggtcaagcagaagctagcggaggtgattctgaaaaaacagcaggcggccctagaaagaacagtccatcccaacagccccggcattccctacagaaccctggagcccctggagacggaaggagccacccgctccatgctcagcagctttttgcctcctgttcccagcctgcccagtgaccccccagagcacttccctctgcgcaagacagtctctgagcccaacctgaagctgcgctataagcccaagaagtccctggagcggaggaagaatccactgctccgaaaggagagtgcgccccccagcctccggcggcggcccgcagagaccctcggagactcctccccaagtagtagcagcacgcccgcatcagggtgcagctcccccaatgacagcgagcacggccccaatcccatcctgggctcggaggctgacagtgaccgcaggacccatccgactctgggccctcgggggccaatcctggggagcccccacactcccctcttcctgccccatggcttggagcccgaggctgggggcaccttgccctctcgcctgcagcccattctcctcctggacccctcaggctctcatgccccgctgctgactgtgcccgggcttgggcccttgcccttccactttgcccagtccttaatgaccaccgagcggctctctgggtcaggcctccactggccactgagccggactcgctcagagcccctgccccccagtgccaccgctcccccaccgccgggccccatgcagccccgcctggagcagctcaaaactcacgtccaggtgatcaagaggtcagccaagccgagtgagaagccccggctgcggcagataccctcggctgaagacctggagacagatggcgggggaccgggccaggtggtggacgatggcctggagcacagggagctgggccatgggcagcctgaggccagaggccccgctcctctccagcagcaccctcaggtgttgctctgggaacagcagcgactggctgggcggctcccccggggcagcaccggggacactgtgctgcttcctctggcccagggtgggcaccggcctctgtcccgggctcagtcttccccagccgcacctgcctcactgtcagccccagagcctgccagccaggcccgagtcctctccagctcagagacccctgccaggaccctgcccttcaccacagggctgatctatgactcggtcatgctgaagcaccagtgctcctgcggtgacaacagcaggcacccggagcacgccggccgcatccagagcatctggtcccggctgcaggagcgggggctccggagccagtgtgagtgtctccgaggccggaaggcctccctggaagagctgcagtcggtccactctgagcggcacgtgctcctctacggcaccaacccgctcagccgcctcaaactggacaacgggaagctggcagggctcctggcacagcggatgtttgtgatgctgccctgtggtggggttggggtggacactgacaccatctggaatgagcttcattcctccaatgcagcccgctgggccgctggcagtgtcactgacctcgccttcaaagtggcttctcgtgagctaaagaatggtttcgctgtggtgcggcccccaggacaccatgcagatcattcaacagccatgggcttctgcttcttcaactcagtggccatcgcctgccggcagctgcaacagcagagcaaggccagcaagatcctcattgtagactgggacgtgcaccatggcaacggcacccagcaaaccttctaccaagaccccagtgtgctctacatctccctgcatcgccatgacgacggcaacttcttcccggggagtggggctgtggatgaggtaggggctggcagcggtgagggcttcaatgtcaatgtggcctgggctggaggtctggacccccccatgggggatcctgagtacctggctgctttcaggatagtcgtgatgcccatcgcccgagagttctctccagacctagtcctggtgtctgctggatttgatgctgctgagggtcacccggccccactgggtggctaccatgtttctgccaaatgttttggatacatgacgcagcaactgatgaacctggcaggaggcgcagtggtgctggccttggagggtggccatgacctcacagccatctgtgacgcctctgaggcctgtgtggctgctcttctgggtaacagggtggatcccctttcagaagaaggctggaaacagaaacccaacctcaatgccatccgctctctggaggccgtgatccgggtgcacagtaaatactggggctgcatgcagcgcctggcctcctgtccagactcctgggtgcctagagtgccaggggctgacaaagaagaagtggaggcagtgaccgcactggcgtccctctctgtgggcatcctggctgaagataggccctcggagcagctggtggaggaggaagaacctatgaatctctaaggctctggaaccatctgcccgcccaccatgcccttgggacctggttctcttctaacccctggcaatagcccccattcctgggtctttagagatcctgtgggcaagtagttggaaccagagaacagcctgcctgctttgacagttatcccagggagcgtgagaaaatccctgggtctagaatgggaactggagaggaccctgagaggagacgggctgggcggcgacccccacagggctctcgagaacagattctcccctccagtatgggccctggctgtggcccccattcctcaggactgcacagaggaggactggctccggctccgtcgggctcacccttaaccactattcctggctctgcaaaccccagactttgcacacagcctcaggctccacacagaaatgtgaacttggcctcagacaggctggcccttcctaggctctaggggctaggggggagtggggagccaagaggtcccatattcctgagtgcaggggtagtccctctcacctgcttcctcagacgactctggaagcttccctctaccactgggcactgagacgaagctccctgacagccgagactggcagccctccatctggtccgtaccctcgccagaggcccccctacatcaacctcctggcgatgccctggtggagcagatgggtgctctgggagtcctgtgcttcctgatccaatggtgccaaacccttcatctccccaagaagcgcagcatacccctgggacccctcggccactgcccactcggggagccttctctgtttctggggcctcccccaccatagctctgattcccaccccacataggagtagcctgactgagggggaaggggtgggagagaagatacagacatggaggaggggaggctgctctggcaaagtcttcaaggcttttgggggtccaggcctggggtcaagaaggaaaatgtgtgtgagcatgtgtgtgagtgaggcgtgtgtgtgagcgtgtgtgtgagtgaggcgtgtgtgtgtgtctttcctaggacccaccataccctgtgtatgtatgcatgtttttgtaaaaaggaagaaaatggaaaaaaatctgaacaataaatgttttatttgctttaaaa

(19) United States
(12) Patent Application Publication
(10) Pub. No.: US 2024/0226074 A1
(43) Pub. Date: Jul. 11, 2024

(54) CELL-OF-ORIGIN TARGETED DRUG REPURPOSING FOR TRIPLE-NEGATIVE AND INFLAMMATORY BREAST CARCINOMA WITH HDAC AND HSP90 INHIBITORS COMBINED WITH NICLOSAMIDE

(71) Applicant: Tan A. Ince, Greenbrook, NJ (US)

(72) Inventor: Tan A. Ince, Greenbrook, NJ (US)

(21) Appl. No.: 18/540,647

(22) Filed: Dec. 14, 2023

Related U.S. Application Data
(60) Provisional application No. 63/387,475, filed on Dec. 14, 2022.

(51) Int. Cl. A61K 31/437; A61K 31/167; A61K 31/18; A61K 31/395; A61K 31/4406; A61K 45/06; A61P 35/00; C12Q 1/6886

(52) U.S. Cl. CPC A61K 31/437; A61K 31/167; A61K 31/18; A61K 31/395; A61K 31/4406; A61K 45/06; A61P 35/00; C12Q 1/6886; C12Q 2600/136; C12Q 2600/158

(57) ABSTRACT

Accurate methods for detecting cancer and for determining the prognosis of cancer, including breast cancer, are described herein, using biomarkers referred to herein as the ET-29, HDAC1, HDAC7, and ZNF92. Upregulation of HDAC1, HDAC7, and ZNF92 and optionally one or more genes in ET-29 biomarkers predicts sensitivity to a three-drug combination that includes histone deacetylase inhibitor(s) (HDAC), heat shock protein 90 (HSP90) inhibitor(s), and helminth(s).

### CROSS REFERENCE TO RELATED APPLICATIONS

This application is a continuation of U.S. Provisional Application Ser. No. 63/387,475, filed Dec. 14, 2022, the contents of which are specifically incorporated herein by reference in its entirety.

### INCORPORATION BY REFERENCE OF SEQUENCE LISTING

This application contains a Sequence Listing which has been submitted electronically in ST26 format and is hereby incorporated by reference in its entirety. Said ST26 file, created on Apr. 1, 2024, is named “1676192US1.xml” and is 62,496 bytes in size.

### BACKGROUND

Breast cancer is one of the most prevalent malignancies with an estimated 2.3 million new cases and 685,000 deaths per year globally. Approximately 60 to 70% of breast cancers express the estrogen receptor (ER+), 10 to 15% are ERBB2-amplified (HER2+) and 10 to 15% are triple-negative breast cancers (TNBC) that are ER−, PR−, and Her2−. While there are targeted therapies available for ER+ and HER2+ breast cancer, no targeted therapy is available for TNBC or IBC, which have a worse outcome than other subtypes.

The major cause of death in all subtypes of breast cancer is metastatic spread of the cancer cells through lymphatics and blood vessels to other organs. A very aggressive and lethal clinical type of cancer, named inflammatory breast cancer (IBC), is more common in women of African-American descent, younger than 40, and overweight patients and is associated with extensive dermal lympho-vascular invasion (LVI) and high metastatic risk. It is worth mentioning that the term pseudo-inflammatory may have been more accurate for this entity; since the edema, erythema and tenderness that mimics breast inflammation (mastitis) is due to dermal tumor emboli plugging the lympho-vascular circulation, rather than true inflammation. Nevertheless, IBC is a very difficult challenge in the clinic; despite the distinct biology of IBC there is no specific therapy for this disease. The patients with IBC are treated with systemic treatments similar to other non-inflammatory breast cancers^{[1]}.

The example of breast cancer suggests that targeted therapies can leverage both acquired targets (HER2 amplification) and intrinsic targets (ER expression). The breast cancer acquired amplification of ERBB2 has been targeted with anti-HER2 antibody, biologics and small molecule ERBB2 kinase inhibitors. In the case of hormonally driven breast cancer, the over-expression of ER seems to be a reflection of the normal cell-origin or differentiation lineage since ER mutations or amplifications are exceedingly rare in untreated patients. In some cases, ER mutations emerge after anti-estrogen treatment as a resistance mechanism. Nevertheless, the anti-estrogens that block ER transcriptional activity or suppress estrogen production have been highly-effective in ER+ breast cancer.

### SUMMARY

The human body is composed of hundreds of different normal cell types. Some of the important and unique features of these normal cell types are inherited by the cancer cells that arise from them. Indeed, the activities of the tumor gene mutations are constrained by this normal cell inheritance. For example, it was found that the same oncogenes produce highly malignant tumors in some cell types, but not in others. In this study it is demonstrated that this cell-of-origin difference can be used to develop cell-targeted therapies, which is a novel concept that differs from the gene-targeted therapies. This cell-based approach led to a surprising discovery that a drug that was approved for treating tapeworm infestation together with an antibiotic derivative can significantly enhance killing of breast cancer cells. Since these drugs are already approved for clinical use, it may be possible to repurpose them to treat breast cancer.

Breast cancer gene expression signatures are described herein include ET-29 (29 genes as shown in Tables 2 and 3) that are co-upregulated by HDAC1 and HDAC7 in metastatic TNBC and have a ZNF92 binding site in their promotor. Upregulation of HDAC1, HDAC7, and ZNF92 predicts sensitivity to a 3-drug combination including a Histone Deacetylase (HDAC inhibitor), combined with a heat shock protein 90 (HSP 90) inhibitor and a helminth such as the tape-worm drug Niclosamide. These 3 drugs are synergistic in killing breast cancer cells. Importantly, the two-drug combinations are not synergistic.

Described herein are methods that can include: a. assaying a biological sample from a subject for expression of HDAC1, HDAC7, and ZNF92 genes to determine one or more expression levels for the HDAC1, HDAC7, and ZNF92 genes; b. comparing the determined expression levels with one or more reference values to identify any altered expression levels in the subject's biological sample, wherein altered expression levels of the HDAC1, HDAC7, and ZNF92 genes in the biological sample relative to the reference value indicates that the subject has cancer with poor prognosis or the subject has malignant cancer, and absence of altered expression of the HDAC1, HDAC7, and ZNF92 genes relative to the reference value indicates that the subject does not have a cancer with poor prognosis or does not have malignant cancer; and optionally c. administering one or more histone deacetylase inhibitor(s) (HDAC), one or more inhibitor of heat shock protein 90 (HSP90), and one or more helminths to a subject determined to have a cancer with poor prognosis or a malignant cancer. In one embodiment, the biological sample from the subject is further assayed for expression of one or more genes encoding an amino acid sequence having at least 90% sequence identity to SEQ ID NOs: 3-31. In one embodiment, the sample is a breast cancer sample. In one embodiment, the sample is a cervical cancer sample, uterine cancer, or prostate cancer. In one embodiment, one or more of ML239, Tipifarnib, Tivantinib, CHIR-99021, Tubastatin-A, GDC-0879, SB-525334, PRIMA-1-met, or a combination thereof, is further administered to the subject. In one embodiment, the administering one or more histone deacetylase inhibitor(s), one or more heat shock factor inhibitor, and the one or more helminths are delivered simultaneously. In one embodiment, the sample is a physiological fluid sample, a tumor tissue sample, or a cancer cell sample. In one embodiment, the subject is a human. In one embodiment, the mammal has inflammatory breast cancer. In one embodiment, the mammal has triple negative breast cancer. In one embodiment, RNA expression is assayed. In one embodiment, protein expression is assayed. In one embodiment, the inhibitor of HSP90 comprises an inhibitor of HSP90a and HSP90s. In one embodiment, the inhibitor of HSP90 comprises an antibiotic. In one embodiment, the inhibitor of HSP90 is Pimitespib, Tanespimycin, or a combination thereof. In one embodiment, the helminth is niclosamide. In one embodiment, the at least one HDAC inhibitor is Belinostat, Entinostat, Vorinostat, or a combination thereof.

Described herein are methods that can include preventing, inhibiting or treating cancer in a mammal, comprising: administering to the mammal a composition comprising one or more histone deacetylase inhibitor(s), one or more heat shock factor inhibitor, and one or more helminths, wherein the cancer in the mammal is determined to have altered expression levels of HDAC1, HDAC7, and ZNF92 genes, relative to a reference value. In one embodiment, the biological sample from the subject is further assayed for expression of one or more genes encoding an amino acid sequence having at least 90% sequence identity to SEQ ID NOs: 3-31. In one embodiment, the mammal is a human. In one embodiment, the mammal has breast cancer, cervical cancer, uterine cancer, or prostate cancer. Methods described herein can include (a) contacting cancer cells expressing HDAC1, HDAC7, or ZNF92 genes or producing HDAC1, HDAC7, or ZNF92 proteins with a test agent; (b) measuring HDAC1, HDAC7, or ZNF92 RNA or protein expression levels in the cells or measuring HDAC1, HDAC7, or ZNF92 protein activity levels; and (c) determining that the test agent reduces the expression levels or activity levels of HDAC1, HDAC7, or ZNF92 genes, to thereby identifying a test agent as a candidate agent that reduces HDAC1, HDAC7, or ZNF92 genes expression levels or activity levels. Also described herein is a pharmaceutical composition comprising one or more histone deacetylase inhibitor(s), one or more heat shock factor inhibitor, and one or more helminths.

### BRIEF DESCRIPTION OF THE FIGURES

The figures illustrate generally, by way of example, but not by way of limitation, various embodiments discussed herein.

FIGS. 1A-1C show the identification of metastatic TNBC cell-of-origin targets and drugs. FIG. 1A: The estrogen receptor negative normal breast cells cultures, BPE and HME, were generated from same donor [2]. Transformation of BPE and HME with the same genes gave rise metastatic BPLER and non-metastatic HMLER tumor cell lines with a TNBC phenotypes [2]. The metastatic TNBC BPLER cells are associated with higher expression of HDAC1, HDAC7 and ZNF92, compared to non-metastatic TNBC HMLER cells [25, 27]. FIG. 1B: HDAC1 upregulates HDAC7, which in turn upregulates 29 super-enhancer (SE) associated genes with ZNF92 binding sites (Table 1) [27]. This HDAC1-HDAC7-ZNF92 axis can be targeted by HDAC inhibitors (Entinostat, Belinostat, and Vorinostat). FIG. 1C: ZNF92 expression correlates only with 17-AAG sensitivity (arrow) among 265 small molecules in the Genomics of Drug Sensitivity in Cancer (GDSC) dataset, with IC50 in 860 cell lines and their corresponding mRNA gene expression (red circle, top left). All the other drugs have a negative correlation with ZNF92 (blue circles). GSCA only draws plot for the top 30 ranked drugs; blue filled circles represent negative correlation, red filled circles represent positive correlation, the color intensity reflects higher correlation, the circle size is positively correlated with the false discovery rate (FDR) significance, and the black outline border indicates FDR ≤0.05. See supplemental data for details. FIG. 1D summarizes the correlation between the HDAC1-HDAC7-ZNF92 axis targets including the 29 genes (shown in Table 2) expression signature and the sensitivity of CTRP drugs (top 30), including the IC50 of 481 small molecules in 1001 cell lines and corresponding mRNA gene expression from Genomics of Therapeutics Response Portal (CTRP). The mRNA expression data and drug sensitivity data were merged. Pearson correlation analysis was performed to get the correlation between gene mRNA expression and drug IC50. P-value was adjusted by FDR. Only the top 17 ranked drugs shown, Niclosamide is ranked #2 (arrow). The blue filled circles represent negative correlation, red filled circles represent positive correlation, the color intensity reflects higher correlation, the circle size is positively correlated with the false discovery rate (FDR) significance, and the black outline border indicates FDR ≤0.05.

FIG. 2 shows treatment of triple-negative breast carcinoma with HDAC-I inhibitor Entinostat (E), Niclosamide (N), and HSP90-I inhibitor Tanespimycin (T). The TNBC cell lines BT549, BT20, HCC38, CAL148, HCC 1143, HCC 1913, MDAMB231, MDAMB43, MFM223, Sum159 and Sum1315 are treated with Entinostat (E: 100 nM), Niclosamide (N: 100 nM), and Tanespimycin (T: 50 nM) alone and in combination (ENT) in 96-well plates for 7 days with indicated drugs. The inhibition of cell proliferation by E, N, T and ENT is measured by WST assay. The relative cell number is calculated compared to vehicle control (DMSO) and expressed as mean±SD (n=6) of percent viability. (*) Inhibition in cell proliferation greater than expected by additivity.

FIGS. 3A-3B shows treatment of triple-negative breast carcinoma with HDAC-I inhibitors Vorinostat (V) or Belinostat (B), with Niclosamide (N), and HSP90-I Pimitespib (P). The TNBC cell lines BT20, MDAMB231, MDAMB435 and Sum159 are cultured in 96-well plates for 7 days with indicated drugs. The inhibition of cell proliferation is measured by WST assay. The relative cell numbers are calculated compared to vehicle control (DMSO) and expressed as mean±SD (n=6) of percent viability. (A) Vorinostat (V: 1000 nM), Niclosamide (N: 100 nM), and Pimitespib (P: 600 nM), and (B) Belinostat (B: 1000 nM), Niclosamide (N: 100 nM) and Pimitespib (P: 400 nM). (*) Inhibition in cell proliferation greater than expected by additivity.

FIGS. 4A-4D show triple-negative breast carcinoma dose response and test combination of HDAC inhibitor Entinostat (E), Niclosamide (N), and HSP90 inhibitor Tanespimycin (T). All experiments were performed in 96-well plates for 7 days. DMSO was used as vehicle control. Inhibition of cell proliferation is measured by the WST assay. Each value is expressed as mean t SD (n=6) of percent viability. (*) Inhibition in cell proliferation greater than expected by additivity. FIG. 4A: Dose response plots of Entinostat (E), Niclosamide (N) and Tanespimycin (T) in BPLER-TNBC cells (0, 15, 32.5, 65, 125, 250, 500, 1000, 2500, 5000, 10000 nM). FIG. 4B: Dose response plots of Entinostat (E), Niclosamide (N) and Tanespimycin (T) in BT20, MDAMB231 and Sum159 TNBC cells lines (0, 15, 32.5, 65, 125, 250, 500, 1000, 2500, 5000, 10000 nM). Highlighted regions in the graphs show the range of doses used for three-drug synergy experiments. FIG. 4C: Three drug combination of Entinostat (E: 100 nM), Niclosamide (N: 100 nM) and Tanespimycin (T: 30 nM), abbreviated as “ENT”, in BLER cells. FIG. 4D: Three drug combination of Entinostat (250 nM: Sum159, 100 nM: MDA-MB231, 50 nM: BT20), Niclosamide (250 nM: Sum159, 100 nM: MDA-MB231, 50 nM: BT20), and Tanespimycin (50 nM: Sum 159 and MDA-MB231, 30 nM:BT20).

FIGS. 5A-5B. HSP90 inhibitor Pimitespib (P) and HDAC inhibitors Vorinostat (V) and Belinostat (B). FIG. 5A: Dose response plots of HSP90 inhibitor Pimitespib (P) and HDAC inhibitors Vorinostat (V) and Belinostat (B) in BPLER-TNBC cells (0, 15, 32.5, 65, 125, 250, 500, 1000, 2500, 5000, and 10000 nM). FIG. 5A: Three drug combination of Entinostat (50 nM for BT20 and 100 nM for all other cell lines), Niclosamide (N: 100 nM), and Pimitespib (P: 400 nM). FIG. 5B: The cells are cultured in 96-well plates for 7 days with drug. Inhibition of cell proliferation is measured by WST assay, the relative cell numbers are calculated compared to vehicle control (DMSO) and expressed as mean f SD (n=6) of percent viability. (*) Inhibition in cell proliferation greater than expected by additivity.

FIGS. 6A-6C. Triple-negative breast carcinoma triple-drug combinations of HDAC inhibitor Vorinostat (V), Belinostat (B) or Entinostat (E), with Niclosamide (N) and HSP90-I Pimitespib (P). The TNBC cell lines BT20, MDAMB231, MDAMB435 and Sum159 are cultured in 96-well plates for 7 days with drug. Inhibition of cell proliferation is measured by WST assay, the relative cell numbers are calculated compared to vehicle control (DMSO) and expressed as mean f SD (n=6) of percent viability. (*) Inhibition in cell proliferation greater than expected by additivity. See supplementary table 1 for details. FIG. 6A: Vorinostat (V: 1000 nM), Niclosamide (N. 100 nM), and Pimitespib (P: 200 nM). FIG. 6B: Belinostat (B: 1000 nM), Niclosamide (N: 100 nM), and Pimitespib (P: 200 nM). FIG. 6C: Entinostat (E: 100 nM), Niclosamide (N: 100 nM), and Pimitespib (P: 200 nM).

FIGS. 7A-7C. Inflammatory breast cancer triple-drug combinations. The combination of HDAC inhibitor Entinostat (E) and Belinostat (B) with Niclosamide (N) and HSP90 inhibitor Pimitespib (P) in inflammatory breast cancer (IBC) cell lines. The IBC03, SUM-149 and SUM-190 cell lines are cultured in F12 medium (Gibco 11765-054) and KPL-4 is cultured in Ham's F12 medium (Corning 10-080-CV) with the indicated drugs. The inhibition of cell proliferation is measured by WST assay, the relative cell numbers are calculated compared to vehicle control (DMSO) and expressed as mean±SD (n=6) of percent viability. (*) Inhibition in cell proliferation greater than expected by additivity. FIG. 7A: Dose response plots of HDAC inhibitor Entinostat (E), Niclosamide (N) and Pimitespib (P) in IBC cell lines (0, 15, 32.5, 65, 125, 250, 500, 1000, 2500, 5000, and 10000 nM). FIG. 7B: Entinostat (100 or 150 nM), alone or combined with Pimitespib (100 or 200 nM), and Niclosamide (100 nM). FIG. 7C: Belinostat (500 or 1000 nM), alone or combined with Pimitespib (100 or 200 nM), and Niclosamide (100 nM).

FIG. 8. Frequency of genomic alterations in the cell-of-origin signature. The image illustrates that frequency of mutations, structural variants and copy number changes in HDAC1, HDAC7, ZNF92 and the 29 genes with the ZNF92 binding sites (excluding alterations of unknown significance). The analysis is carried out in the cBioPortal combined TCGA and Metabric datasets with 3,593 breast cancer samples and shows that none of the genes in the cell-origin associated HDAC1-HDAC7-ZNF92 axis and their 29 downstream targets have bona fide driver mutations in more than 0.4% of breast cancer (https://www.cbioportal.org/).[1, 2]

### DETAILED DESCRIPTION

As illustrated herein, breast cancer gene expression signatures referred to herein as ET-29 (29 genes as shown in Tables 2 and 3), together with HDAC1, HDAC7 and ZNF92 genes predicts sensitivity of triple-negative breast cancer (TNBC) to a 3-drug combination including a Histone Deacetylase (HDAC inhibitor), combined with a heat shock protein 90 (HSP 90) inhibitor and a tape-worm drug Niclosamide. These 3 drugs are synergistic in killing breast cancer cells. Expression of ET-29, together with HDAC1, HDAC7 and ZNF92 genes, are useful markers for detecting, diagnosing, and determining the prognosis of cancer, including breast cancer. Methods for detecting, diagnosing, and determining the prognosis of cancer, including breast cancer, are also described herein.

The markers are a cell-of-origin specific mRNA signature associated with the over-expression of histone deacetylases and zinc finger protein and metastasis and poor outcome in triple-negative breast cancer (TNBC). Based on this signature, it was discovered that the combination of three drugs: a histone deacetylase (HDAC) inhibitor, an anti-helminthic Niclosamide, and an antibiotic Tanespimycin that inhibits heat shock protein 90 (HSP90) synergistically reduces the proliferation of all twelve TNBC cell lines that were tested.

Additionally, it was discovered that four out of five inflammatory breast cancer (IBC) cells are sensitive to this combination. Significantly, the concentration of the drugs that are used in these experiments are within or below clinically achievable dose, and the synergistic activity only emerged when all three drugs were combined. The present results indicate that combining HDAC and HSP90 inhibitors with the tapeworm drug Niclosamide can achieve remarkably synergistic inhibition of TNBC and IBC. Since Niclosamide, HDAC and HSP90 inhibitors were approved for clinical use for other cancer types, it may be possible to repurpose their combination for TNBC and IBC.

Historically, three or more drugs have been routinely combined for many cancer treatments^{[49]}. Partly due to the difficulty of screening high-order combinations, new drug candidates are often tested in combination with first-line treatments, sometimes without a compelling hypothesis^{[50-56]}. As expected, most of the 54 breast clinical trials with HDAC and HSP90 inhibitors have been in combination with anti-estrogen, anti-HER2 and/or chemotherapy drugs, and we have not found any breast cancer trials for niclosamide (Table 1).

TABLE 1
Breast cancer clinical trials with HDAC and HSP90 inhibitors.

| | | | | Drug 2 | | |
|---|---|---|---|---|---|---|
| Rank | NCT Number | Drug 1 | Drug 2 | target | Phase | Status |
| 1 | NCT00413075 | Belinostat | | | Phase 1 | Completed |
| 2 | NCT00413322 | Belinostat | 5-FU | DNA | Phase 1 | Completed |
| | | | | synthesis | | |
| 3 | NCT04315233 | Belinostat | Ribociclib | Cyclin D1/ | Phase 1 | Recruiting |
| | | | | CDK4/6 | | |
| 4 | NCT04703920 | Belinostat | Talazoparib | PARP | Phase 1 | Recruiting |
| 5 | NCT03432741 | Belinostat | | | Phase 1 | Recruiting |
| 7 | NCT00627627 | Belinostat | | | Phase 1\|2 | Withdrawn |
| 6 | NCT00817362 | Belinostat | Trastuzumab | HER2 | Phase 2 | Terminated |
| 8 | NCT02453620 | Entinostat | Nivolumab | PD-L1/2 | Phase 1 | Active, not |
| | | | | | | recruiting |
| 13 | NCT02833155 | Entinostat | | | Phase 1 | Completed |
| 14 | NCT02820961 | Entinostat | Exemestane | ER | Phase 1 | Completed |
| 15 | NCT01594398 | Entinostat | | | Phase 1 | Completed |
| 16 | NCT01434303 | Entinostat | Lapatinib | HER2 | Phase 1 | Completed |
| 17 | NCT02623751 | Entinostat | | | Phase 1 | Completed |
| 18 | NCT02897778 | Entinostat | | | Phase 1 | Completed |
| 19 | NCT00020579 | Entinostat | | | Phase 1 | Completed |
| 24 | NCT03473639 | Entinostat | | | Phase 1 | Recruiting |
| 26 | NCT04296942 | Entinostat | Adotrastuzumab | HER2 | Phase 1 | Terminated |
| 27 | NCT00754312 | Entinostat | | | Phase 1 | Terminated |
| 20 | NCT02708680 | Entinostat | Atezolizumab | PD-L1 | Phase 1\| 2 | Completed |
| 9 | NCT03280563 | Entinostat | | | Phase 1\|2 | Active, not |
| | | | | | | recruiting |
| 10 | NCT01349959 | Entinostat | Azacitidine | DNA | Phase 2 | Active, not |
| | | | | methyl- | | recruiting |
| | | | | transferase | | |
| 21 | NCT00828854 | Entinostat | | | Phase 2 | Completed |
| 22 | NCT00676663 | Entinostat | Exemestane | ER | Phase 2 | Completed |
| 23 | NCT03291886 | Entinostat | | | Phase 2 | Completed |
| 25 | NCT03361800 | Entinostat | | | Phase 2 | Terminated |
| 28 | NCT01234532 | Entinostat | Anastrozole | ER | Phase 2 | Terminated |
| 29 | NCT02115594 | Entinostat | | | Phase 2 | Withdrawn |
| 11 | NCT03538171 | Entinostat | | | Phase 3 | Active, not |
| | | | | | | recruiting |
| 12 | NCT02115282 | Entinostat | | | Phase 3 | Active, not |
| | | | | | | recruiting |
| 30 | NCT00004065 | Tanespimycin | | | Phase 1 | Completed |
| 31 | NCT00773344 | Tanespimycin | Trastuzumab | HER2 | Phase 1\|2 | Completed |
| 32 | NCT00096109 | Tanespimycin | | | Phase 2 | Terminated |
| 34 | NCT01720602 | Vorinostat | | | N/A | Completed |
| 35 | NCT01153672 | Vorinostat | | | N/A | Completed |
| 55 | NCT01655004 | Vorinostat | | | N/A | Unknown |
| | | | | | | status |
| 57 | NCT01695057 | Vorinostat | | | N/A | Withdrawn |
| 36 | NCT00719875 | Vorinostat | Capecitabine | DNA | Phase 1 | Completed |
| | | | | synthesis | | |
| 37 | NCT01084057 | Vorinostat | Ixabepilone | Microtubules | Phase 1 | Completed |
| 38 | NCT00788112 | Vorinostat | | | Phase 1 | Completed |
| 39 | NCT00838929 | Vorinostat | Radiation | | Phase 1 | Completed |
| 40 | NCT00045006 | Vorinostat | | | Phase 1 | Completed |
| 46 | NCT03742245 | Vorinostat | Olaparib | PARP | Phase 1 | Recruiting |
| 47 | NCT03878524 | Vorinostat | | | Phase 1 | Recruiting |
| 49 | NCT01249443 | Vorinostat | Carboplatin | | Phase 1 | Terminated |
| 41 | NCT00574587 | Vorinostat | Chemotherapy | | Phase 1\|2 | Completed |
| 42 | NCT00368875 | Vorinostat | Paclitaxel | Microtubules | Phase 1\|2 | Completed |
| 43 | NCT00258349 | Vorinostat | Trastuzumab | HER2 | Phase 1\|2 | Completed |
| 50 | NCT01118975 | Vorinostat | Lapatinib | HER2 | Phase 1\|2 | Terminated |
| 56 | NCT00416130 | Vorinostat | | | Phase 1\|2 | Unknown |
| | | | | | | status |
| 33 | NCT00616967 | Vorinostat | Carboplatin | | Phase 2 | Active, not |
| | | | | | | recruiting |
| 44 | NCT00365599 | Vorinostat | Tamoxifen | ER | Phase 2 | Completed |
| 45 | NCT00262834 | Vorinostat | | | Phase 2 | Completed |
| 48 | NCT04190056 | Vorinostat | Pembrolizumab | PD-L1 | Phase 2 | Recruiting |
| 51 | NCT01194427 | Vorinostat | Tamoxifen | ER | Phase 2 | Terminated |
| 52 | NCT00132002 | Vorinostat | | | Phase 2 | Terminated |
| 53 | NCT02395627 | Vorinostat | | | Phase 2 | Terminated |
| 54 | NCT00126451 | Vorinostat | | | Phase 2 | Terminated |

However, these combinations do not necessarily originate with high-order experimental synergy screens, which are currently unfeasible due to exponential increase in complexity to analyze the interaction of more than two drugs^{[57]}. For example, the Gene Set Cancer Analysis (GSCA) we used in this study integrates >10,000 genomic data sets and 750 drugs from GDSC and CTRP, which represents more than 70 million possibilities for 3-drug combinations. Thus, a systematic three drug biological screen was not feasible even with 20 candidates, because it would require testing 1,140 three-drug combinations^{[57]}.

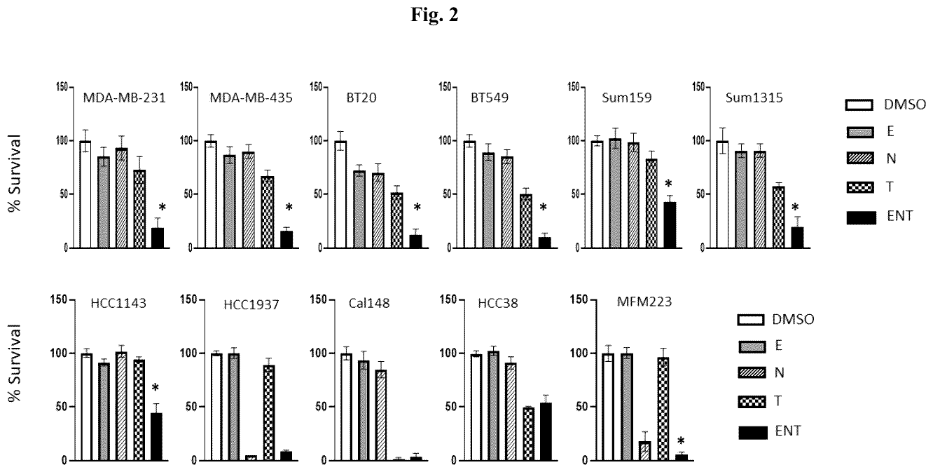

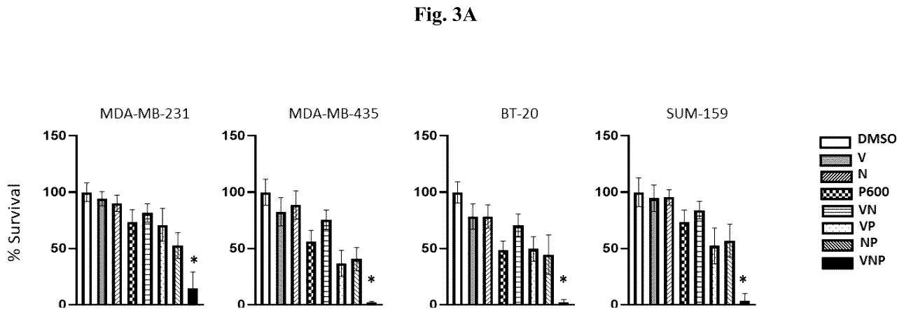

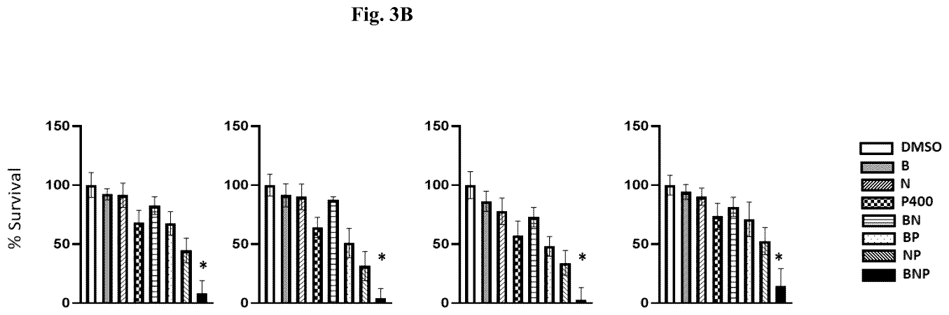

As a solution, it is thought that pairwise interaction scores can provide reliable estimates for three-drug interactions 11. However, the pairwise results of HDAC-L, HSP90-L, and niclosamide were not significantly additive. FIGS. 3A-3B. Described herein is the de novo discovery of the three-drug synergy of the HDAC-I, HSP90-I and niclosamide combination, which would not have been discovered empirically without a cell-of-origin hypothesis-based approach[⁵⁹]. The three-drug synergy of HDAC-I inhibitor, HSP90-I inhibitor, and Niclosamide is demonstrated in FIGS. 2, 3A, 3B, 4C, 4Dd, 5B, 6A-6C, 7B, and 7C.

### Samples

Breast cancer can be assessed through the evaluation of expression patterns, or profiles, of the HDAC1, HDAC7 and ZNF92 genes, and optionally one or more genes in the ET-29 gene mRNA signature shown in Tables 2 and 3, in one or more subject samples. The term subject, or subject sample, refers to an individual regardless of health and/or disease status. A subject can be a subject, a study participant, a control subject, a screening subject, or any other class of individual from whom a sample is obtained and assessed using the markers and/or methods described herein. Accordingly, a subject can be diagnosed with breast cancer, can present with one or more symptoms of breast cancer, or a predisposing factor, such as a family (genetic) or medical history (medical) factor, for breast cancer, can be undergoing treatment or therapy for breast cancer, or the like. Alternatively, a subject can be healthy with respect to any of the aforementioned factors or criteria. It will be appreciated that the term “healthy” as used herein, is relative to breast cancer status, as the term “healthy” cannot be defined to correspond to any absolute evaluation or status. Thus, an individual defined as healthy with reference to any specified disease or disease criterion, can in fact be diagnosed with any other one or more diseases, or exhibit any other one or more disease criterion, including one or more cancers other than breast cancer. However, the healthy controls are preferably free of any cancer.

In some cases, the methods for detecting, predicting, and/or assessing the prognosis of breast cancer include collecting a biological sample comprising a cell or tissue, such as a breast tissue sample or a primary breast tumor tissue sample. By “biological sample” is intended any sampling of cells, tissues, or bodily fluids in which expression of the ET-29, HDAC1, HDAC7 and ZNF92 genes can be detected. Examples of such biological samples include, but are not limited to, biopsies and smears. Bodily fluids useful in the present invention include blood, lymph, urine, saliva, nipple aspirates, gynecological fluids, or any other bodily secretion or derivative thereof. Blood can include whole blood, plasma, serum, or any derivative of blood. In some embodiments, the biological sample includes breast cells, particularly breast tissue from a biopsy, such as a breast tumor tissue sample. Biological samples may be obtained from a subject by a variety of techniques including, for example, by scraping or swabbing an area, by using a needle to aspirate cells or bodily fluids, or by removing a tissue sample (i.e., biopsy). In some embodiments, a breast tissue sample is obtained by, for example, fine needle aspiration biopsy, core needle biopsy, or excisional biopsy.

The samples can be stabilized for evaluating and/or quantifying ET-29, HDAC1, HDAC7 and ZNF92 genes expression levels.

In some cases, fixative and staining solutions may be applied to some of the cells or tissues for preserving the specimen and for facilitating examination. Biological samples, particularly breast tissue samples, may be transferred to a glass slide for viewing under magnification. In one embodiment, the biological sample is a formalin-fixed, paraffin-embedded breast tissue sample, particularly a primary breast tumor sample.

### Gene Expression

Various methods can be used for evaluating and/or quantifying ET-29, HDAC1, HDAC7 and ZNF92 expression levels. “Evaluating and/or quantifying” is intended to determine the quantity or presence of an RNA transcript or its expression product of ET-29, HDAC1, HDAC7 and ZNF92 genes.

TABLE 2
The ET-29 gene mRNA signature downstream of the HDACI-HDAC7-ZNF92
axis.

| | Entrez ID | Gene Symbol | Gene Name |
|---|---|---|---|
| 1 | 80325 | ABTB1 | ankyrin repeat and BTB domain containing 1 |
| 2 | 9289 | ADGRG1 | adhesion G protein-coupled receptor G1 |
| 3 | 113451 | AZIN2 | antizyme inhibitor 2 |
| 4 | 55653 | BCAS4 | breast carcinoma amplified sequence 4 |
| 5 | 79934 | COQ8B | coenzyme Q8B |
| 6 | 9696 | CROCC | ciliary rootlet coiled-coil, rootletin |
| 7 | 1523 | CUX1 | cut like homeobox 1 |
| 8 | 27122 | DKK3 | dickkopf WNT signaling pathway inhibitor 3 |
| 9 | 1891 | ECH1 | enoyl-CoA hydratase 1 |
| 10 | 2049 | EPHB3 | EPH receptor B3 |
| 11 | 23149 | FCHO1 | FCH and mu domain containing endocytic adaptor 1 |
| 12 | 84929 | FIBCD1 | fibrinogen C domain containing 1 |
| 13 | 81544 | GDPD5 | glycerophosphodiester phosphodiesterase domain |
| 14 | 3855 | KRT7 | keratin 7 |
| 15 | 3985 | LIMK2 | LIM domain kinase 2 |
| 16 | 114783 | LMTK3 | lemur tyrosine kinase 3 |
| 17 | 4016 | LOXL1 | lysyl oxidase like 1 |
| 18 | 4430 | MYO1B | myosin IB |
| 19 | 5097 | PCDH1 | protocadherin 1 |
| 20 | 9124 | PDLIM1 | PDZ and LIM domain 1 |
| 21 | 8398 | PLA2G6 | phospholipase A2 group VI |
| 22 | 5875 | RABGGTA | Rab geranylgeranyltransferase subunit alpha |
| 23 | 6337 | SCNN1A | sodium channel epithelial 1 subunit alpha |
| 24 | 92799 | SHKBP1 | SH3KBP1 binding protein 1 |
| 25 | 55315 | SLC29A3 | solute carrier family 29 member 3 |
| 26 | 56848 | SPHK2 | sphingosine kinase 2 |
| 27 | 79816 | TLE6 | TLE family member 6, subcortical maternal |
| 28 | 79639 | TMEM53 | transmembrane protein 53 |
| 29 | 641649 | TMEM91 | transmembrane protein 91 |

Methods for detecting expression of the HDAC1, HDAC7, and ZNF92 genes, and optionally the ET-29 gene mRNA signature, including gene expression profiling, can involve methods based on hybridization analysis of polynucleotides, methods based on sequencing of polynucleotides, immunohistochemistry methods, and proteomics-based methods. The methods generally involve detecting expression products (e.g., mRNA or proteins) of the HDAC1, HDAC7, and ZNF92 genes, and optionally one or more of the ET-29 genes. The methods include detecting expression products of any number of the ET-29 genes including 1, 2, 3, 4, 5, 6, 7, 8, 9, 10, 11, 12, 13, 14, 15, 16, 17, 18, 19, 20, 21, 22, 23, 24, 25, 26, 27, 28, or 29 of the ET-29 genes.

In some cases, PCR-based methods, which can include reverse transcription PCR (RT-PCR) (Weis et al., TIG 8:263-64, 1992), array-based methods such as microarray (Schena et al., Science 270:467-70, 1995), or combinations thereof are used. By “microarray” is intended an ordered arrangement of hybridizable array elements, such as, for example, polynucleotide probes, on a substrate. The term “probe” refers to any molecule that is capable of selectively binding to a specifically intended target biomolecule, for example, a nucleotide transcript or a protein encoded by or corresponding to the ET-29, HDAC1, HDAC7 and ZNF92 genes. Probes can be synthesized or obtained from the ET-29, HDAC1, HDAC7 and ZNF92 nucleic acids or they can be derived from appropriate biological preparations. Probes may be specifically designed to be labeled. Examples of molecules that can be utilized as probes include, but are not limited to, RNA, DNA, proteins, antibodies, and organic molecules.

Many expression detection methods use isolated RNA. The starting material is typically total RNA isolated from a biological sample, such as a cell or tissue sample, a tumor or tumor cell line, a corresponding normal tissue or cell line, or a combination thereof. If the source of RNA is a sample from a subject, RNA (e.g., mRNA) can be extracted, for example, from stabilized, frozen or archived paraffin-embedded, or fixed (e.g., formalin-fixed) tissue samples (e.g., pathologist-guided tissue core samples).

General methods for RNA extraction are available and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In some cases, RNA isolation can be performed using a purification kit, a buffer set and protease from commercial manufacturers, such as Qiagen (Valencia, Calif.), according to the manufacturer's instructions. For example, total RNA from cells can be isolated using Qiagen RNeasy mini-columns. Other commercially available RNA isolation kits include MASTERPURE™ Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from tissue or cell samples (e.g. tumors) can be isolated, for example, by cesium chloride density gradient centrifugation. Additionally, large numbers of tissue samples can readily be processed using available techniques, such as, for example, the single-step RNA isolation process of Chomczynski (U.S. Pat. No. 4,843,155).

Isolated RNA can be used in hybridization or amplification assays that include, but are not limited to, PCR analyses and probe arrays. One method for the detection of RNA levels involves contacting the isolated RNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 60, 100, 250, or 500 nucleotides in length and sufficient to specifically hybridize under stringent conditions to any of the ET-29, HDAC1, HDAC7 and ZNF92 genes, or any derivative DNA or RNA. Hybridization of an mRNA with the probe indicates that the ET-29, HDAC1, HDAC7 and ZNF92 genes in question is being expressed.

In cases, the mRNA from the sample is immobilized on a solid surface and contacted with a probe, for example by running the isolated mRNA on an agarose gel and transferring the mRNA from the gel to a membrane, such as nitrocellulose. In other cases, the probes are immobilized on a solid surface and the mRNA is contacted with the probes, for example, in an Agilent gene chip array. A skilled artisan can readily adapt available mRNA detection methods for use in detecting the level of expression of the ET-29, HDAC1, HDAC7 and ZNF92 genes.

An alternative method for determining the level of the ET-29, HDAC1, HDAC7 and ZNF92 gene expression in a sample involves the process of nucleic acid amplification of the ET-29, HDAC1, HDAC7 and ZNF92 mRNA (or cDNA thereof), for example, by RT-PCR (U.S. Pat. No. 4,683,202), ligase chain reaction (Barany, Proc. Natl. Acad. Sci. USA 88:189-93, 1991), self-sustained sequence replication (Guatelli et al., Proc. Natl. Acad. Sci. USA 87:1874-78, 1990), transcriptional amplification system (Kwoh et al., Proc. Natl. Acad. Sci. USA 86:1173-77, 1989), Q-Beta Replicase (Lizardi et al., Bio/Technology 6:1197, 1988), rolling circle replication (U.S. Pat. No. 5,854,033), or any other nucleic acid amplification method, followed by the detection of the amplified molecules using available techniques. These detection schemes are especially useful for the detection of nucleic acid molecules if such molecules are present in very low numbers.

In some cases, the ET-29, HDAC1, HDAC7 and ZNF92 gene expression is assessed by quantitative RT-PCR. Numerous different PCR or QPCR protocols are available and can be directly applied or adapted for use using the ET-29, HDAC1, HDAC7 and ZNF92 genes. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR. However, preferred are cyclers with real-time fluorescence measurement capabilities, for example, SMARTCYCLER® (Cepheid, Sunnyvale, Calif.), ABI PRISM 7700® (Applied Biosystems, Foster City, Calif.), ROTOR-GENE™ (Corbett Research, Sydney, Australia), LIGHTCYCLER® (Roche Diagnostics Corp, Indianapolis, Ind.), ICYCLER® (Biorad Laboratories, Hercules, Calif.) and MX4000® (Stratagene, La Jolla, Calif.).

Quantitative PCR (QPCR) (also referred as real-time PCR) is preferred under some circumstances because it provides not only a quantitative measurement, but also reduced time and contamination. In some instances, the availability of full gene expression profiling techniques is limited due to requirements for fresh frozen tissue and specialized laboratory equipment, making the routine use of such technologies difficult in a clinical setting. However, QPCR gene measurement can be applied to standard formalin-fixed paraffin-embedded clinical tumor blocks, such as those used in archival tissue banks and routine surgical pathology specimens (Cronin et al. (2007) Clin Chem 53:1084-91)[Mullins 2007] [Paik 2004]. As used herein, “quantitative PCR (or “real time QPCR”) refers to the direct monitoring of the progress of PCR amplification as it is occurring without the need for repeated sampling of the reaction products. In quantitative PCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time.

In some cases, microarrays are used for expression profiling. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316. High-density oligonucleotide arrays are particularly useful for determining the gene expression profile for a large number of RNAs in a sample. Techniques for the synthesis of these arrays using mechanical synthesis methods are described in, for example, U.S. Pat. No. 5,384,261. Although a planar array surface can be used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992. Arrays can be packaged in such a manner as to allow for diagnostics or other manipulation of an all-inclusive device. See, for example, U.S. Pat. Nos. 5,856,174 and 5,922,591.

When using microarray techniques, PCR amplified inserts of cDNA clones can be applied to a substrate in a dense array. The microarrayed genes, immobilized on the microchip, are suitable for hybridization under stringent conditions. Fluorescently labeled cDNA probes can be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance.

With dual color fluorescence, separately labeled cDNA probes generated from two sources of RNA can be hybridized pairwise to the array. The relative abundance of the transcripts from the two sources corresponding to each specified gene is thus determined simultaneously. A miniaturized scale can be used for the hybridization, which provides convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93:106-49, 1996). Microarray analysis can be performed by commercially available equipment, following manufacturer's protocols, such as by using the Affymetrix GenChip technology, or Agilent ink jet microarray technology. The development of microarray methods for large-scale analysis of gene expression makes it possible to search systematically for molecular markers of cancer classification and outcome prediction in a variety of tumor types.

As used herein “level”, refers to a measure of the amount of, or a concentration of a transcription product, for instance an mRNA, or a translation product, for instance a protein or polypeptide.

As used herein “activity” refers to a measure of the ability of a transcription product or a translation product to produce a biological effect or to a measure of a level of biologically active molecules.

As used herein “expression level” further refer to gene expression levels or gene activity. Gene expression can be defined as the utilization of the information contained in a gene by transcription and translation leading to the production of a gene product.

The terms “increased,” or “increase” in connection with expression of the biomarkers described herein generally means an increase by a statically significant amount. For the avoidance of any doubt, the terms “increased” or “increase” means an increase of at least 10% as compared to a reference value, for example an increase of at least about 20/6, or at least about 30%, or at least about 40%, or at least about 50%, or at least about 60%, or at least about 70%, or at least about 80%, or at least about 90% or up to and including a 100% increase or any increase between 10-100% as compared to a reference value or level, or at least about a 1.5-fold, at least about a 1.6-fold, at least about a 1.7-fold, at least about a 1.8-fold, at least about a 1.9-fold, at least about a 2-fold, at least about a 3-fold, or at least about a 4-fold, or at least about a 5-fold, at least about a 10-fold increase, any increase between 2-fold and 10-fold, at least about a 25-fold increase, or greater as compared to a reference level. In some embodiments, an increase is at least about 1.8-fold increase over a reference value.

Similarly, the terms “decrease,” or “reduced,” or “reduction,” or “inhibit” in connection with expression of the biomarkers described herein generally to refer to a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level or non-detectable level as compared to a reference sample), or any decrease between 10-100% as compared to a reference level.

A “reference value” is a predetermined reference level, such as an average or median of expression levels of each of the ET-29, HDAC1, HDAC7 and ZNF92 genes in, for example, biological samples from a population of healthy subjects. The reference value can be an average or median of expression levels of each of the ET-29, HDAC1, HDAC7 and ZNF92 genes in a chronological age group matched with the chronological age of the tested subject. In some embodiments, the reference biological samples can also be gender matched. In some embodiments, the reference biological samples can also be cancer containing tissue from a specific subgroup of patients, such as stage 1, stage 2, stage 3, or grade 1, grade 2, grade 3 cancers, non-metastatic cancers, untreated cancers, hormone treatment resistant cancers, HER2 amplified cancers, triple negative cancers, estrogen negative cancers, or other relevant biological or prognostic subsets. For example, as explained herein, malignancy associated response signature expression levels in a sample can be assessed relative to normal breast tissue from the same subject or from a sample from another subject or from a repository of normal subject samples. If the expression level of a biomarker is greater or less than that of the reference or the average expression level, the biomarker expression is said to be “increased” or “decreased,” respectively, as those terms are defined herein. Exemplary analytical methods for classifying expression of a biomarker, determining a malignancy associated response signature status, and scoring of a sample for expression of a malignancy associated response signature biomarker are explained in detail herein.

### Treatment

Methods are described herein for treating cancer. Such methods can involve administering therapeutic agents that can treat cancers with poor prognosis. Examples of such therapeutic agents can include one or more histone deacetylase inhibitor (HDAC), ZNF92 inhibitor, histone demethylase inhibitor, mTOR inhibitor, polo-like kinase (PLK) inhibitor, heat shock factor inhibitor, and/or inhibitors of any of the ET-9 and/or ET-60 breast cancer cell-origin associated signature biomarkers described herein.

In some cases, the cancer includes breast cancer, ovarian cancer, colon cancer, brain cancer, pancreatic cancer, prostate cancer, lung cancer, or melanoma. In some embodiments, the cancer includes leukemia, myeloma, or lymphoma.

The methods include downregulating expression of the ET-29, HDAC1, HDAC7 and ZNF92 genes. Suitable methods for downregulating such expression can include: inhibiting transcription of mRNA; degrading mRNA by methods including, but not limited to, the use of interfering RNA (RNAi); blocking translation of mRNA by methods including, but not limited to, the use of antisense nucleic acids or ribozymes, or the like. In some embodiments, a suitable method for downregulating expression may include providing to the cancer a small interfering RNA (siRNA) targeted to the ET-29, HDAC1, HDAC7 and ZNF92 genes.

Suitable methods for down-regulating the function or activity of the ET-29, HDAC1, HDAC7 and ZNF92 genes, may include administering a small molecule inhibitor that inhibits the function or activity of any of these markers or factors.

In some cases, one or more histone deacetylase inhibitors can be administered to treat cancers with poor prognosis, such as cancers identified by measuring and/or monitoring the ET-29, HDAC1, HDAC7 and ZNF92 genes described herein. In some cases, histone deacetylase inhibitors are not administered to treat cancers with poor prognosis, such as cancers identified by measuring and/or monitoring the ET-29, HDAC1, HDAC7 and ZNF92 genes described herein.

As used herein a “Histone Deacetylase inhibitor” or “HDAC inhibitor” refers to inhibitors of Histone Deacetylase 1 (HDAC1), Histone Deacetylase 7 (HDAC7), and/or phosphorylated HDAC7, including agents that inhibit the level and/or activity of HDAC1 and/or HDAC7 and/or phosphorylated HDAC7, as well as agents that inhibit the phosphorylation of HDAC7 e.g., inhibitors of EMK protein kinase, C-TAK1 protein kinase, and/or CAMK protein kinase, and agents that activate or increase the level and/or activity of phosphatase activity to remove phosphoryl groups from HDAC7, e.g., activators of PP2A phosphatase and/or myosin phosphatase. In some cases, HDAC inhibitors include molecules that bind directly to a functional region of HDAC1 and/or HDAC7 and/or phosphorylated HDAC7 in a manner that interferes with the enzymatic activity of HDAC1 and/or HDAC7 and/or phosphorylated HDAC7 e.g., agents that interfere with substrate binding to HDAC1 and/or HDAC7 and/or phosphorylated HDAC7. In some embodiments, HDAC inhibitors include molecules that bind directly to HDAC7 in a manner that prevents the phosphorylation of HDAC7. HDAC inhibitors include agents that inhibit the activity of peptides, polypeptides, or proteins that modulate the activity of HDAC1 and/or HDAC7 e.g., inhibitors of EMK protein kinase, C-TAK1 kinase, CAMK protein kinase inhibitors of C-TAK1 protein kinase. Examples of suitable inhibitors include, but are not limited to antisense oligonucleotides, oligopeptides, interfering RNA e.g., small interfering RNA (siRNA), small hairpin RNA (shRNA), aptamers, ribozymes, small molecule inhibitors, or antibodies or fragments thereof, and combinations thereof.

In some cases, HDAC inhibitors are specific inhibitors or specifically inhibit the level and/or activity of HDAC1 and/or HDAC7 and/or phosphorylated HDAC7. As used herein, “specific inhibitor(s)” refers to inhibitors characterized by their ability to bind to with high affinity and high specificity to HDAC1 and/or HDAC7 and/or phosphorylated HDAC7 proteins or domains, motifs, or fragments thereof, or variants thereof, and preferably have little or no binding affinity for non-HDAC1 and/or non-HDAC7 and/or non-phosphorylated HDAC7 proteins. As used herein, “specifically inhibit(s)” refers to the ability of an HDAC inhibitor of the present invention to inhibit the level and/or activity of a target polypeptide, e.g., HDAC1, and/or HDAC7, and/or phosphorylated HDAC7, and/or EMK protein kinase, and/or C-TAK1 protein kinase and/or CAMK protein kinase and preferably have little or no inhibitory effect on non-target polypeptides. As used herein, “specifically activate(s)” and “specifically increase(s)” refers to the ability of an HDAC inhibitor of the present invention to stimulate (e.g., activate or increase) the level and/or activity of a target polypeptide, e.g., PP2A phosphatase and/or myosin phosphatase and preferably to have little or no stimulatory effect on non-target polypeptides.

Examples of HDAC inhibitors include Vorinostat (SAHA), Entinostat (MS-275), Panobinostat (LBH589), Trichostatin A (TSA), Mocetinostat (MGCD0103), 4-Phenylbutyric acid (4-PBA), ACY-775, Belinostat (PXD101), Romidepsin (FK228, Depsipeptide), MC1568, Tubastatin A HCl, Givinostat (ITF2357), Dacinostat (LAQ824), CUDC-101, Quisinostat (JNJ-26481585) 2HCl, Pracinostat (SB939), PCI-34051, Droxinostat, Abexinostat (PCI-24781), RGFP966, AR-42, Ricolinostat (ACY-1215), Valproic Acid (NSC 93819) sodium salt, Tacedinaline (CI994), Fimepinostat (CUDC-907), Sodium butyrate, Curcumin, M344, Tubacin, RG2833 (RGFP109), Resminostat, Divalproex Sodium, Scriptaid, Sodium Phenylbutyrate, Tubastatin A, Tubastatin A TFA, Sinapinic Acid, TMP269, Santacruzamate A (CAY10683), TMP195, Valproic acid (VPA), UF010, Tasquinimod, SKLB-23bb, Isoguanosine, NKL22, Sulforaphane, BRD73954, BG45, Domatinostat (4SC-202), Citarinostat (ACY-241), Suberohydroxamic acid, BRD3308, Splitomicin, HPOB, LMK-235, Biphenyl-4-sulfonyl chloride, Nexturastat A, BML-210 (CAY10433), TC-H 106, SR-4370, TH34, Tucidinostat (Chidamide), SIS17, (−)-Parthenolide, WT161, CAY10603, ACY-738, Raddeanin A, GSK3117391, Tinostamustine(EDO-S101), or combinations thereof. Such HDAC inhibitors are available from Selleckchem.com.

In some cases, one or more histone demethylase inhibitors can be administered to treat cancers with poor prognosis, such as cancers identified by measuring and/or monitoring the ET-29, HDAC1, HDAC7 and ZNF92 genes described herein.

In some cases, an inhibitor of histone demethylase can be administered to treat cancers with poor prognosis, such as cancers identified by measuring and/or monitoring the ET-29, HDAC1, HDAC7 and ZNF92 biomarkers described herein. Histone demethylase inhibitors can include GSK-J4, 2,4-Pyridinedicarboxylic Acid, AS8351, Clorgyline hydrochloride, CPI-455, Daminozide, GSK-2879552, GSK-J1, GSK-J2, GSK-J5, GSK-LSD1, IOX1, IOX2, JIB-04, ML-324, NCGC00244536, OG-L002, ORY-1001, SP-2509, TC-E 5002, UNC-926, β-Lapachone, or combinations thereof. Such inhibitors are available, e.g., from Selleckchem.com.

In some cases, one or more mTOR inhibitors can be administered to treat cancers with poor prognosis, such as cancers identified by measuring and/or monitoring the ET-29, HDAC1, HDAC7 and ZNF92 genes described herein. Examples of mTOR inhibitors include Rapamycin (AY-22989), Everolimus (RAD001), AZD8055, Temsirolimus (CCI-779), PI-103, NU7441 (KU-57788), KU-0063794, Torkinib (PP242), Ridaforolimus (Deforolimus, MK-8669), Sapanisertib (MLNO128), Voxtalisib (XL765) Analogue, Torin 1, Omipalisib (GSK2126458), OSI-027, PF-04691502, Apitolisib (GDC-0980), GSK1059615, WYE-354, Gedatolisib (PKI-587), Vistusertib (AZD2014), Torin 2, WYE-125132 (WYE-132), BGT226 (NVP-BGT226) maleate, Palomid 529 (P529), PP121, WYE-687, Clemastine (HS-592) fumarate, Nitazoxanide (NSC 697855), WAY-600, ETP-46464, GDC-0349, PI3K/Akt Inhibitor Library, 4EGI-1, XL388, MHY1485, 3-Hydroxyanthranilic acid, Bimiralisib (PQR309), Samotolisib (LY3023414), Lanatoside C, Rotundic acid, L-Leucine, Chrysophanic Acid, Voxtalisib (XL765), GNE-477, CZ415, Astragaloside IV, CC-115, Salidroside, Compound 401, 3BDO, Zotarolimus (ABT-578), GNE-493, Paxalisib (GDC-0084), Onatasertib (CC 223), ABTL-0812, PQR620, SF2523, Niclosamide, or combinations thereof. Such HDAC inhibitors are available from Selleckchem.com.

In some cases, one or more Polo-Like Kinase (PLK) inhibitors can be administered to treat cancers with poor prognosis, such as cancers identified by measuring and/or monitoring the ET-29, HDAC1, HDAC7 and ZNF92 genes described herein. Examples of PLK inhibitors include BI 2536, Volasertib (BI 6727), Wortmannin (KY 12420), Rigosertib (ON-01910), GSK461364, HMN-214, MLN0905, Ro3280, SBE 13 HCl, Centrinone (LCR-263), CFI-400945, HMN-176, Onvansertib (NMS-P937), or combinations thereof.

In some cases, one or more heat shock factor inhibitors can be administered to treat cancers with poor prognosis, such as cancers identified by measuring and/or monitoring the expression of the ET-29, HDAC1, HDAC7 and ZNF92 genes described herein. Examples of heat shock factor inhibitors include one or more of the following Tanespimycin (17-AAG), Pimitespib (TAS-116, Luminespib (NVP-AUY922), Alvespimycin (17-DMAG) HCl, Ganetespib (STA-9090), Onalespib (AT13387), Geldanamycin (NSC 122750), SNX-2112 (PF-04928473), PF-04929113 (SNX-5422), KW-2478, Cucurbitacin D, VER155008, VER-50589, CH5138303, VER-49009, NMS-E973, Zelavespib (PU-H71), HSP990 (NVP-HSP990), XL888 NVP-BEP800, BIIB021 or a combination thereof. Such heat shock factor inhibitors can be obtained from Tocris.com.

As used herein, “solid tumor” is intended to include, but not be limited to, the following sarcomas and carcinomas: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. Solid tumor is also intended to encompass epithelial cancers.

In embodiments, the ET-29, HDAC1, HDAC7 and ZNF92 biomarkers comprise a nucleic acid sequence or amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or less than 100% nucleic acid sequence identity or amino acid sequence identity to a nucleic acid sequence or an amino acid sequence having any of SEQ ID NOs: 3-31.

Zinc Finger Protein (ZNF92) ZNF92 is a zinc finger protein that functions as transcription factor that binds nucleic acids and regulates transcription. The ZNF92 gene is located on chromosome 7 (Gene ID: 168374; location NC_000007.14 (65373855..65401136). An example of an amino acid sequence for ZNF92 isoform 1 is available as UNIPROT accession no. Q03936-1 and shown below as SEQ ID NO:1.

```
                  10 20 30 40
MGPLTFRDVK IEFSLEEWQC LDTAQRNLYR DVMLENYRNL
                50 60 70 80
VFLGIAVSKP DLITWLEQGK EPWNLKRHEM VDKTPVMCSH
                90 100 110 120
FAQDVWPEHS IKDSFQKVIL RTYGKYGHEN LQLRKDHKSV
              130 140 150 160
DACKVYKGGY NGLNQCLTTT DSKIFQCDKY VKVFHKFPNV
              170 180 190 200
NRNKIRHTGK KPFKCKNRGK SFCMLSQLTQ HKKIHTREYS
              210 220 230 240
YKCEECGKAF NWSSTLTKHK IIHTGEKPYK CEECGKAFNR
              250 260 270 280
SSNLTKHKII HTGEKPYKCE ECGKAFNRSS TLTKHKRIHT
              290 300 310 320
EEKPYKCEEC GKAFNQFSIL NKHKRIHMED KPYKCEECGK
              330 340 350 360
AFRVFSILKK HKIIHTGEKP YKCEECGKAF NQFSNLTKHK
              370 380 390 400
IIHTGEKPYK CDECGKAFNQ SSTLTKHKRI HTGEKPYKCE
              410 420 430 440
ECGKAFKQSS TLTEHKIIHT GEKPYKCEKC GKAFSWSSAF
              450 460 470 480
TKHKRNHMED KPYKCEECGK AFSVFSTLTK HKIIHTREKP
              490 500 510 520
YKCEECGKAF NQSSIFTKHK IIHTEGKSYK CEKCGNAFNQ
              530 540 550 560
SSNLTARKII YTGEKPYKYE ECDKAFNKFS TLITHQIIYT
              570 580
GEKPCKHECG RAFNKSSNYT KEKLQT
```

A cDNA sequence encoding the amino acid sequence of SEQ ID NO:1 ZNF92 protein is available as NCBI accession no. BC040594.1, shown below as SEQ ID NO:

| | |
|---|---|
| 1 | CTCTCGCTGC AGCCGGCGCT CCACGTCTAG TCTTCACTGC |
| 41 | TCTGCGTCCT GTGCTGATAA AGGCTCGCCG CTGTGACCCT |
| 81 | GTTACCTGCA AGAACTTGGA GGTTCACAGC TAAGACGCCA |
| 121 | GGACCCCCTG GAAGCCTAGA AATGGGACCA CTGACATTTA |
| 161 | GGGATGTGAA AATAGAATTC TCTCTAGAGG AATGGCAATG |
| 201 | CCTGGACACT GCGCAGCGGA ATTTATATAG AGATGTGATG |
| 241 | TTAGAGAACT ACAGAAACCT GGTCTTCCTT GGTATTGCTG |
| 281 | TCTCTAAGCC AGACCTGATC ACCTGGCTGG AGCAAGGAAA |
| 321 | AGAGCCCTGG AATCTGAAGA GACATGAGAT GGTAGACAAA |
| 361 | ACCCCAGTTA TGTGTTCTCA TTTTGCCCAA GATGTTTGGC |
| 401 | CAGAGCACAG CATAAAAGAT TCTTTCCAAA AAGTGATACT |
| 441 | GAGAACATAT GGAAAATATG GACATGAGAA TTTACAGCTA |
| 481 | AGAAAAGACC ATAAAAGTGT GGATGCATGT AAGGTGTACA |
| 521 | AAGGAGGTTA TAATGGACTT AACCAGTGTT TGACAACTAC |
| 561 | TGACAGCAAG ATATTTCAGT GTGATAAATA TGTGAAAGTC |
| 601 | TTTCATAAAT TTCCAAATGT AAATAGAAAT AAGATAAGAC |
| 641 | ATACTGGAAA GAAACCTTTC AAATGTAAAA ACCGTGGCAA |
| 681 | ATCATTTTGC ATGCTTTCAC AATTAACTCA ACATAAGAAA |
| 721 | ATTCATACTA GAGAGTATTC TTACAAATGT GAAGAATGTG |
| 761 | GTAAAGCCTT TAACTGGTCC TCAACCCTTA CTAAACATAA |
| 801 | GATAATTCAT ACTGGAGAAA AACCCTACAA ATGTGAAGAA |
| 841 | TGTGGCAAAG CTTTTAACCG GTCCTCAAAT CTTACTAAAC |
| 881 | ATAAAATAAT TCATACTGGA GAGAAACCCT ACAAATGTGA |
| 921 | AGAATGTGGC AAAGCTTTTA ACCGGTCCTC AACCCTTACT |
| 961 | AAACATAAAA GAATTCATAC AGAAGAGAAA CCCTACAAAT |
| 1001 | GTGAAGAATG TGGCAAGGCC TTTAACCAGT TCTCGATTCT |
| 1041 | TAATAAACAT AAGAGAATTC ATATGGAAGA TAAACCCTAC |
| 1081 | AAATGTGAAG AATGTGGCAA AGCCTTTAGA GTATTCTCAA |
| 1121 | TTCTTAAAAA ACATAAGATA ATCCATACTG GGGAAAAACC |
| 1161 | ATACAAATGT GAAGAATGTG GCAAAGCCTT TAACCAGTTC |
| 1201 | TCAAACCTTA CTAAACATAA GATAATTCAT ACTGGAGAGA |
| 1241 | AACCCTACAA ATGTGATGAA TGTGGCAAAG CCTTTAACCA |
| 1281 | GTCCTCAACC CTTACTAAAC ATAAAAGAAT TCATACGGGA |
| 1321 | GAAAAACCCT ACAAATGTGA AGAATGTGGC AAAGCTTTTA |
| 1361 | AACAGTCCTC AACCCTTACT GAACATAAGA TAATTCATAC |
| 1401 | TGGAGAGAAA CCCTACAAAT GTGAAAAATG TGGCAAGGCC |
| 1441 | TTTAGCTGGT CCTCAGCTTT TACTAAACAT AAGAGAAATC |
| 1481 | ATATGGAAGA TAAACCCTAC AAATGTGAAG AATGTGGCAA |
| 1521 | AGCCTTTAGT GTATTCTCAA CCCTTACTAA ACATAAAATA |
| 1561 | ATTCATACTA GAGAAAAACC CTACAAATGT GAAGAATGTG |
| 1601 | GCAAAGCCTT TAACCAGTCC TCAATTTTTA CTAAACATAA |
| 1641 | GATAATTCAC ACTGAAGGGA AATCCTACAA ATGTGAAAAA |
| 1681 | TGTGGCAATG CTTTTAACCA GTCCTCAAAC CTTACTGCAC |
| 1721 | GTAAGATAAT TTATACTGGA GAGAAACCCT ACAAATATGA |
| 1761 | AGAATGTGAC AAAGCCTTTA ACAAGTTCTC AACCCTTATT |
| 1801 | ACACATCAGA TAATTTATAC TGGAGAGAAA CCCTGCAAAC |
| 1841 | ATGAATGTGG CAGAGCCTTT AACAAATCCT CAAATTATAC |
| 1881 | TAAAGAGAAA CTACAAACCT GAAAGATGTG ACAATGATTT |
| 1921 | TCACTACACC TCAAACTTTT CTAAACATAA ACCATATTGG |
| 1961 | TGCCCTAGAA ATGTGAGGAA TATGACAAGG ACTTTAAATG |
| 2001 | GTTGTCACGC TTGATTGTAG GTAAGATAAT TTATATTGGA |
| 2041 | GAAAAATCCT CCAAGTATGA AGAATGTGGC AAACTTTTAA |
| 2081 | CCAATCCTCA CACCTTATTG CACAGGAAAG CATTTATACT |
| 2121 | TGAGAAAAAT TGTATAAAGA ATATGGAAAA GCCATTTATA |
| 2161 | TCTGCTCACA TGTAAAAACA TCAGTTCATA CTTAATAAAA |
| 2201 | TGCAATTACC GTCAAATCTT TCAGAAAATA TAAGCCTTTA |
| 2241 | ATACGAGGAA GAGTATTCTT AAGATGAACA TTACAAATAG |
| 2281 | AAAGAGGGTT GTAGTACCTT TAGTTTTATG ATAGATCTTA |
| 2321 | TTGTACACAT TTTGTACCAG AGGAAAACCC TAAAGCATTA |
| 2361 | GTTGCTCAAA CTTTGTTCGA CATCAGGGAA TTTGTATTGG |
| 2401 | AGAAAAACCC TGCAAATGTA ATAAATATGG AAAAACATTT |
| 2441 | TTTCAAAAAC TACAGCTTGG AAAACATCAG AGAGTTCATA |
| 2481 | CTAAAATATA TTTTTGCAGA TGCAGTAAAT ATGAAAAATA |
| 2521 | TTTAATCCCA AATTAAGTCT ATGTAAATAT CAGAATTCAC |
| 2561 | AGTAGAAATC ATAAGGCATA AGGCACTGAT ACTTCAGACA |
| 2601 | TTACACTAAA TTAGAGTGTT GAGTATAGGA GATCCAAAAC |
| 2641 | TAAAATTGTT AGGTAAGTTA TTTATATATA ACTTTAAAAG |
| 2681 | AAGTAGAAGA TTTTTTGGAG ATTTATAATT ACATTCAAAG |
| 2721 | TATACTTTTT TCTTGAAAAA AATTACAGAT TTTTTGAAAA |
| 2761 | GCAATTGATG TAATTTAACT CTCAAATTCA TGTTTTTCTT |
| 2801 | CATTCCTATT ATATTCACAT GTGAAAGCAA GTGATCTGTT |
| 2841 | GTTGCTGAAT CAGAGATATG AGAGATTCTT TTTTATAGGT |
| 2881 | GGGCATTATT TATGCCCCTT TCTGTGGAAG AGTAAGAAAA |
| 2921 | TTAAAATACA AGATGCATGA GGAAAATGTA GAGATGCTCT |
| 2961 | TTGTGATTAA CTTAGAATAT TAAGTGCTAC TTGACGTACA |
| 3001 | TGTTCAGACT AACATTCTTT TGCAGTATAG TGAGAAAAAA |
| 3041 | ACATTTTAAA ATTAATTATC ATTTTGTTGA TTGTGCTTTT |
| 3081 | ATGTAATAAA ATGCAGTACT TTAAAACAAA AAAAAAAAAA |
| 3121 | AAA |

The ET-29 signature (shown in Table 2) genes are listed below in Table 3 with UNIPROT accession numbers and examples of amino acid sequences.

### Table 3: ET-29 Signature (Shown in Table 2)

TABLE 3
The ET-29 gene mRNA signature downstream of the HDAC1-HDAC7-ZNF92
axis.

| Entrez | ET-29 subset Name & |
|---|---|
| ID | Example of Human Amino Acid Sequence |
| 80325 | ABTB1 (ankyrin repeat and BTB domain containing 1; SEQ ID NO: 3) |
| Uniprot | MDTSDLFASCRKGDVGRVRYLLEQRDVEVNVRDKWDSTPLYYACLCGHEELVLYLLANGA |
| Q969K4 | RCEANTEDGERCLYGALSDPIRRALRDYKQVTASCRRRDYYDDFLQRLLEQGIHSDVVFV |
| | VHGKPFRVHRCVLGARSAYFANMLDTKWKGKSVVVLRHPLINPVAFGALLQYLYTGRLDI |
| | GVEHVSDCERLAKQCQLWDLLSDLEAKCEKVSEFVASKPGTCVKVITIEPPPADPRLRED |
| | MALLADCALPPELRGDLWELPFPCPDGENSCPDICERVAGCSFLCHKAFFCGRSDYFRAL |
| | LDDHFRESEEPATSGGPPAVILHGISPDVFTHVLYYMYSDHTELSPEAAYDVLSVADMYL |
| | LPGLKRLCGRSLAQMLDEDTVVGVWRVAKLERLARLEDQCTEYMAKVIEKLVEREDEVEA |
| | VKEEAAAVAARQETDSIPLVDDIRFHVASTVQTYSAIEEAQQRLRALEDLLVSIGLDC |
| 9289 | ADGRG1 (adhesion G protein-coupled receptor G1; SEQ ID NO: 4) |
| Uniprot | MTPQSLLQTTLFLLSLLELVQGAHGRGHREDFRFCSQRNQTHRSSLHYKPTPDLRISIEN |
| Q9Y653 | SEEALTVHAPFPAAHPASRSFPDPRGLYHFCLYWNRHAGRLHLLYGKRDFLLSDKASSLL |
| | CFQHQEESLAQGPPLLATSVTSWWSPQNISLPSAASFTFSFHSPPHTAAHNASVDMCELK |
| | RDLQLLSQFLKHPQKASRRPSAAPASQQLQSLESKLTSVREMGDMVSFEEDRINATVWKL |
| | QPTAGLQDLHIHSRQEEEQSEIMEYSVLLPRTLFQRTKGRSGEAEKRLLLVDESSQALFQ |
| | DKNSSQVLGEKVLGIVVQNTKVANLTEPVVLTFQHQLQPKNVTLQCVFWVEDPTLSSPGH |
| | WSSAGCETVRRETQTSCFCNHLTYFAVLMVSSVEVDAVHKHYLSLLSYVGCVVSALACLV |
| | TIAAYLCSRVPLPCRRKPRDYTIKVHMNLLLAVELLDTSELLSEPVALTGSEAGCRASAI |
| | FLHESLLTCLSWMGLEGYNLYRLVVEVFGTYVPGYLLKLSAMGWGFPIFLVTLVALVDVD |
| | NYGPIILAVHRTPEGVIYPSMCWIRDSLVSYITNLGLFSLVELENMAMLATMVVQILRLR |
| | PHTQKWSHVITLLGLSLVLGLPWALIFFSFASGTFQLVVLYLFSIITSFQGFLIFIWYWS |
| | MRLQARGGPSPLKSNSDSARLPISSGSTSSSRI |
| 113451 | AZIN2 (antizyme inhibitor 2; SEQ ID NO: 5) |
| Uniprot | MAGYLSESDEVMVEEGESTRDLLKELTLGASQATTDEVAAFFVADLGAIVRKHFCFLKCL |
| Q96A70 | PRVRPFYAVKCNSSPGVLKVLAQLGLGFSCANKAEMELVQHIGIPASKIICANPCKQIAQ |
| | IKYAAKHGIQLLSEDNEMELAKVVKSHPSAKMVLCIATDDSHSLSCLSLKFGVSLKSCRH |
| | LLENAKKHHVEVVGVSFHIGSGCPDPQAYAQSIADARLVFEMGTELGHKMHVLDLGGGFP |
| | GTEGAKVRFEEIASVINSALDLYFPEGCGVDIFAELGRYYVTSAFTVAVSIIAKKEVLLD |
| | QPGREEENGSTSKTIVYHLDEGVYGIFNSVLEDNICPTPILQKKPSTEQPLYSSSLWGPA |
| | VDGCDCVAEGLWLPQLHVGDWLVFDNMGAYTVGMGSPFWGTQACHITYAMSRVAWEALRR |
| | QLMAAEQEDDVEGVCKPLSCGWEITDTLCVGPVETPASIM |
| 55653 | BCAS4 (breast carcinoma amplified sequence 4; SEQ ID NO: 6) |
| Uniprot | MQRTGGGAPRPGRNHGLPGSLRQPDPVALLMLLVDADQPEPMRSGARELALFLTPEPGAE |
| Q8TDM0 | AKEVEETIEGMLLRLEEFCSLADLIRSDTSQILEENIPVLKAKLTEMRGIYAKVDRLEAF |
| | VKMVGHHVAFLEADVLQAERDHGAFPQALRRWLGSAGLPSERNVECSGTIPARCNLRLPG |
| | SSDSPASASQVAGITEVTCTGARDVRAAHTV |
| 79934 | COQ8B (coenzyme Q8B; SEQ ID NO: 7) |
| Uniprot | MWLKVGGLLRGTGGQLGQTVGWPCGALGPGPHRWGPCGGSWAQKFYQDGPGRGLGEEDIR |
| Q96D53 | RAREARPRKTPRPQLSDRSRERKVPASRISRLANFGGLAVGLGLGVLAEMAKKSMPGGRL |
| | QSEGGSGLDSSPFLSEANAERIVQTLCTVRGAALKVGQMLSIQDNSFISPQLQHIFERVR |
| | QSADFMPRWQMLRVLEEELGRDWQAKVASLEEVPFAAASIGQVHQGLLRDGTEVAVKIQY |
| | PGIAQSIQSDVQNLLAVLKMSAALPAGLFAEQSLQALQQELAWECDYRREAACAQNERQL |
| | LANDPFFRVPAVVKELCTTRVLGMELAGGVPLDQCQGLSQDLRNQICFQLLTLCLRELFE |
| | FRFMQTDPNWANFLYDASSHQVILLDEGASREFGTEFTDHYIEVVKAAADGDRDCVLQKS |
| | RDLKELTGFETKAFSDAHVEAVMILGEPFATQGPYDFGSGETARRIQDLIPVLLRHRLCP |
| | PPEETYALHRKLAGAFLACAHLRAHIACRDLFQDTYHRYWASRQPDAATAGSLPTKGDSW |
| | VDPS |
| 9696 | CROCC (ciliary rootlet coiled-coil, rootletin; SEQ ID NO: 8) |
| Uniprot | MSLGLAGAQEVELTLETVIQTLESSVLCQEKGLGARDLAQDAQITSLPALIREIVTRNLS |
| Q5TZA2 | QPESPVLLPATEMASLLSLQEENQLLQQELSRVEDLLAQSRAERDELAIKYNAVSERLEQ |
| | ALRLEPGELETQEPRGLVRQSVELRRQLQEEQASYRRKLQAYQEGQQRQAQLVQRLQGKI |
| | LQYKKRCSELEQQLLERSGELEQQRLRDTEHSQDLESALIRLEEEQQRSASLAQVNAMLR |
| | EQLDQAGSANQALSEDIRKVINDWTRCRKELEHREAAWRREEESFNAYFSNEHSRLLLLW |
| | RQVVGFRRLVSEVKMETERDLLQLGGELARTSRAVQEAGLGLSTGLRLAESRAEAALEKQ |
| | ALLQAQLEEQLRDKVLREKDLAQQQMQSDLDKADLSARVTELGLAVKRLEKQNLEKDQVN |
| | KDLTEKLEALESLRLQEQAALETEDGEGLQQTLRDLAQAVLSDSESGVQLSGSERTADAS |
| | NGSLRGLSGQRTPSPPRRSSPGRGRSPRRGPSPACSDSSTLALIHSALHKRQLQVQDMRG |
| | RYEASQDLLGTLRKQLSDSESERRALEEQLQRLRDKTDGAMQAHEDAQREVQRLRSANEL |
| | LSREKSNLAHSLQVAQQQAEELRQEREKLQAAQEELRRQRDRLEEEQEDAVQDGARVRRE |
| | LERSHRQLEQLEGKRSVLAKELVEVREALSRATLQRDMLQAEKAEVAEALTKAEAGRVEL |
| | ELSMTKLRAEEASLQDSLSKLSALNESLAQDKLDLNRLVAQLEEEKSALQGRQRQAEQEA |
| | TVAREEQERLEELRLEQEVARQGLEGSLRVAEQAQEALEQQLPTLRHERSQLQEQLAQLS |
| | RQLSGREQELEQARREAQRQVEALERAAREKEALAKEHAGLAVQLVAAEREGRTLSEEAT |
| | RLRLEKEALEGSLFEVQRQLAQLEARREQLEAEGQALLLAKETLTGELAGLRQQIIATQE |
| | KASLDKELMAQKLVQAEREAQASLREQRAAHEEDLQRLQREKEAAWRELEAERAQLQSQL |
| | QREQEELLARLEAEKEELSEEIAALQQERDEGLLLAESEKQQALSLKESEKTALSEKLMG |
| | TRHSLATISLEMERQKRDAQSRQEQDRSTVNALTSELRDLRAQREEAAAAHAQEVRRLQE |
| | QARDLGKQRDSCIREAEELRTQLRLLEDARDGLRRELLEAQRKLRESQEGREVQRQEAGE |
| | LRRSLGEGAKEREALRRSNEELRSAVKKAESERISLKLANEDKEQKLALLEEARTAVGKE |
| | AGELRTGLQEVERSRLEARRELQELRRQMKMLDSENTRLGRELAELQGRLALGERAEKES |
| | RRETLGLRQRLLKGEASLEVMRQELQVAQRKLQEQEGEFRTRERRLLGSLEEARGTEKQQ |
| | LDHARGLELKLEAARAEAAELGIRLSAAEGRAQGLEAELARVEVQRRAAEAQLGGLRSAL |
| | RRGLGLGRAPSPAPRPVPGSPARDAPAEGSGEGINSPSTLECSPGSQPPSPGPATSPASP |
| | DLDPEAVRGALREFLQELRSAQRERDELRTQTSALNRQLAEMEAERDSATSRARQLQKAV |
| | AESEEARRSVDGRLSGVQAELALQEESVRRSERERRATLDQVATLERSLQATESELRASQ |
| | EKISKMKANETKLEGDKRRLKEVLDASESRTVKLELQRRSLEGELQRSRLGLSDREAQAQ |
| | ALQDRVDSLQRQVADSEVKAGTLQLTVERINGALAKVEESEGALRDKVRGLTEALAQSSA |
| | SLNSTRDKNLHLQKALTACEHDRQVLQERIDAARQALSEARKQSSSIGEQVQTLRGEVAD |
| | LELQRVEAEGQLQQLREVLRQRQEGEAAALNTVQKLQDERRLLQERLGSLQRALAQLEAE |
| | KREVERSALRLEKDRVALRRTLDKVEREKLRSHEDTVRLSAEKGRLDRTLTGAELELAEA |
| | QRQIQQLEAQVVVLEQSHSPAQLEVDAQQQQLELQQEVERLRSAQAQTERTLEARERAHR |
| | QRVRGLEEQVSTLKGQLQQELRRSSAPFSPPSGPPEK |
| 1523 | CUX1 (cut like homeobox 1; SEQ ID NO: 9) |
| Uniprot | MLCVAGARLKRELDATATVLANRQDESEQSRKRLIEQSREFKKNTPEDIRKQVAPLIKSE |
| P39880 | QGEIDALSKRSKEAEAAFINVYKRLIDVPDPVPALDLGQQLQLKVQRLHDIETENQKLRE |
| | TLEEYNKEFAEVKNQEVTIKALKEKIREYEQTLKNQAETIALEKEQKLQNDFAEKERKLQ |
| | ETQMSTTSKLEEAEHKVQSLQTALEKTRTELFDLKTKYDEETTAKADEIEMIMTDLERAN |
| | QRAEVAQREAETLREQLSSANHSLQLASQIQKAPDVEQAIEVLTRSSLEVELAAKEREIA |
| | QLVEDVQRLQASLTKLRENSASQISQLEQQLSAKNSTLKQLEEKLKGQADYEEVKKELNI |
| | LKSMEFAPSEGAGTQDAAKPLEVLLLEKNRSLQSENAALRISNSDLSGSARRKGKDQPES |
| | RRPGSLPAPPPSQLPRNPGEQASNINGTHQFSPAGLSQDFFSSSLASPSLPLASTGKFAL |
| | NSLLQRQLMQSFYSKAMQEAGSTSMIFSTGPYSTNSISSQSPLQQSPDVNGMAPSPSQSE |
| | SAGSVSEGEEMDTAEIARQVKEQLIKHNIGQRIFGHYVLGLSQGSVSEILARPKPWNKLT |
| | VRGKEPFHKMKQFLSDEQNILALRSIQGRQRENPGQSLNRLFQEVPKRRNGSEGNITTRI |
| | RASETGSDEAIKSILEQAKRELQVQKTAEPAQPSSASGSGNSDDAIRSILQQARREMEAQ |
| | QAALDPALKQAPLSQSDITILTPKLISTSPMPTVSSYPPLAISLKKPSAAPEAGASALPN |
| | PPALKKEAQDAPGLDPQGAADCAQGVLRQVKNEVGRSGAWKDHWWSAVQPERRNAASSEE |
| | AKAEETGGGKEKGSGGSGGGSQPRAERSQLQGPSSSEYWKEWPSAESPYSQSSELSLTGA |
| | SRSETPQNSPLPSSPIVPMSKPTKPSVPPLTPEQYEVYMYQEVDTIELTRQVKEKLAKNG |
| | ICQRIFGEKVLGLSQGSVSDMLSRPKPWSKITQKGREPFIRMQLWINGELGQGVLPVQGQ |
| | QQGPVLHSVTSLQDPLQQGCVSSESTPKTSASCSPAPESPMSSSESVKSLTELVQQPCPP |
| | IEASKDSKPPEPSDPPASDSQPTTPLPLSGHSALSIQELVAMSPELDTYGITKRVKEVLT |
| | DNNLGQRLFGETILGLTQGSVSDLLARPKPWHKLSLKGREPFVRMQLWINDPNNVEKLMD |
| | MKRMEKKAYMKRRHSSVSDSQPCEPPSVGTEYSQGASPQPQHQLKKPRVVLAPEEKEALK |
| | RAYQQKPYPSPKTIEDLATQLNLKTSTVINWFHNYRSRIRRELFIEEIQAGSQGQAGASD |
| | SPSARSGRAAPSSEGDSCDGVEATEGPGSADTEEPKSQGEAEREEVPRPAEQTEPPPSGT |
| | PGPDDARDDDHEGGPVEGPGPLPSPASATATAAPAAPEDAATSAAAAPGEGPAAPSSAPP |
| | PSNSSSSSAPRRPSSLQSLFGLPEAAGARDSRDNPLRKKKAANLNSIIHRLEKAASREEP |
| | IEWEF |
| 27122 | DKK3 (dickkopf WNT signaling pathway inhibitor 3; SEQ ID NO: 10) |
| Uniprot | MQRLGATLLCLLLAAAVPTAPAPAPTATSAPVKPGPALSYPQEEATLNEMFREVEELMED |
| Q9UBP4 | TQHKLRSAVEEMEAEEAAAKASSEVNLANLPPSYHNETNTDTKVGNNTIHVHREIHKITN |
| | NQTGQMVESETVITSVGDEEGRRSHECIIDEDCGPSMYCQFASFQYTCQPCRGQRMLCTR |
| | DSECCGDQLCVWGHCTKMATRGSNGTICDNQRDCQPGLCCAFQRGLLFPVCTPLPVEGEL |
| | CHDPASRLLDLITWELEPDGALDRCPCASGLLCQPHSHSLVYVCKPTFVGSRDQDGEILL |
| | PREVPDEYEVGSFMEEVRQELEDLERSLTEEMALREPAAAAAALLGGEEI |
| 1891 | ECHI (enoyl-CoA hydratase 1; SEQ ID NO: 11) |
| Uniprot | MAAGIVASRRLRDLLTRRLTGSNYPGLSISLRLTGSSAQEEASGVALGEAPDHSYESLRV |
| Q13011 | TSAQKHVLHVQLNRPNKRNAMNKVFWREMVECENKISRDADCRAVVISGAGKMFTAGIDL |
| | MDMASDILQPKGDDVARISWYLRDIITRYQETENVIERCPKPVIAAVHGGCIGGGVDLVT |
| | ACDIRYCAQDAFFQVKEVDVGLAADVGTLQRLPKVIGNQSLVNELAFTARKMMADEALGS |
| | GLVSRVFPDKEVMLDAALALAAEISSKSPVAVQSTKVNLLYSRDHSVAESLNYVASWNMS |
| | MLQTQDLVKSVQATTENKELKTVTESKL |
| 2049 | EPHB3 (EPH receptor B3; SEQ ID NO: 12) |
| Uniprot | MARARPPPPPSPPPGLLPLLPPLLLLPLLLLPAGCRALEETLMDTKWVTSELAWTSHPES |
| P54753 | GWEEVSGYDEAMNPIRTYQVCNVRESSQNNWLRTGFIWRRDVQRVYVELKFTVRDCNSIP |
| | NIPGSCKETENLFYYEADSDVASASSPFWMENPYVKVDTIAPDESFSRLDAGRVNTKVRS |
| | FGPLSKAGFYLAFQDQGACMSLISVRAFYKKCASTTAGFALFPETLTGAEPTSLVIAPGT |
| | CIPNAVEVSVPLKLYCNGDGEWMVPVGACTCATGHEPAAKESQCRPCPPGSYKAKQGEGP |
| | CLPCPPNSRTTSPAASICTCHNNFYRADSDSADSACTTVPSPPRGVISNVNETSLILEWS |
| | EPRDLGGRDDLLYNVICKKCHGAGGASACSRCDDNVEFVPRQLGLTERRVHISHLLAHTR |
| | YTFEVQAVNGVSGKSPLPPRYAAVNITTNQAAPSEVPTLRLHSSSGSSLTLSWAPPERPN |
| | GVILDYEMKYFEKSEGIASTVTSQMNSVQLDGLRPDARYVVQVRARTVAGYGQYSRPAEF |
| | ETTSERGSGAQQLQEQLPLIVGSATAGLVEVVAVVVIAIVCLRKQRHGSDSEYTEKLQQY |
| | IAPGMKVYIDPFTYEDPNEAVREFAKEIDVSCVKIEEVIGAGEFGEVCRGRLKQPGRREV |
| | FVAIKTLKVGYTERQRRDFLSEASIMGQFDHPNIIRLEGVVTKSRPVMILTEFMENCALD |
| | SFLRENDGQFTVIQLVGMLRGIAAGMKYLSEMNYVHRDLAARNILVNSNLVCKVSDEGLS |
| | RFLEDDPSDPTYTSSLGGKIPIRWTAPEAIAYRKFTSASDVWSYGIVMWEVMSYGERPYW |
| | DMSNQDVINAVEQDYRLPPPMDCPTALHQLMLDCWVRDRNLRPKESQIVNTLDKLIRNAA |
| | SLKVIASAQSGMSQPLLDRTVPDYTTFTTVGDWLDAIKMGRYKESFVSAGFASEDLVAQM |
| | TAEDLLRIGVTLAGHQKKILSSIQDMRLQMNQTLPVQV |
| 23149 | FCHO1 (FCH and mu domain containing endocytic adaptor 1; SEQ ID |
| Uniprot | NO: 13) |
| O14526 | MSYFGEHFWGEKNHGFEVLYHSVKQGPISTKELADFIRERATIEETYSKAMAKLSKLASN |
| | GTPMGTFAPLWEVERVSSDKLALCHLELTRKLQDLIKDVLRYGEEQLKTHKKCKEEVVST |
| | LDAVQVLSGVSQLLPKSRENYLNRCMDQERLRRESTSQKEMDKAETKTKKAAESLRRSVE |
| | KYNSARADFEQKMLDSALRFQAMEETHLRHMKALLGSYAHSVEDTHVQIGQVHEEFKQNI |
| | ENVSVEMLLRKFAESKGTGREKPGPLDFEAYSAAALQEAMKRLRGAKAFRLPGLSRRERE |
| | PEPPAAVDFLEPDSGTCPEVDEEGFTVRPDVTQNSTAEPSRESSSDSDEDDEEPRKFYVH |
| | IKPAPARAPACSPEAAAAQLRATAGSLILPPGPGGTMKRHSSRDAAGKPQRPRSAPRTSS |
| | CAERLQSEEQVSKNLFGPPLESAFDHEDFTGSSSLGFTSSPSPFSSSSPENVEDSGLDSP |
| | SHAAPGPSPDSWVPRPGTPQSPPSCRAPPPEARGIRAPPLPDSPQPLASSPGPWGLEALA |
| | GGDLMPAPADPTAREGLAAPPRRLRSRKVSCPLTRSNGDLSRSLSPSPLGSSAASTALER |
| | PSFLSQTGHGVSRGPSPVVLGSQDALPIATAFTEYVHAYFRGHSPSCLARVTGELTMTFP |
| | AGIVRVFSGTPPPPVLSFRLVHTTAIEHFQPNADLLESDPSQSDPETKDEWLNMAALTEA |
| | LQRQAEQNPTASYYNVVLLRYQFSRPGPQSVPLQLSAHWQCGATLTQVSVEYGYRPGATA |
| | VPTPLINVQILLPVGEPVINVRLQPAATWNLEEKRLTWRLPDVSEAGGSGRLSASWEPLS |
| | GPSTPSPVAAQFTSEGTTLSGVDLELVGSGYRMSLVKRRFATGMYLVSC |
| 84929 | FIBCD1 (fibrinogen C domain containing 1; SEQ ID NO: 14) |
| Uniprot | MVNDRWKTMGGAAQLEDRPRDKPQRPSCGYVLCTVLLALAVLLAVAVTGAVLFINHAHAP |
| Q8N539 | GTAPPPVVSTGAASANSALVIVERADSSHLSILIDPRCPDLTDSFARLESAQASVLQALT |
| | EHQAQPRLVGDQEQELLDTLADQLPRLLARASELQTECMGLRKGHGTLGQGLSALQSEQG |
| | RLIQLLSESQGHMAHLVNSVSDILDALQRDRGLGRPRNKADLQRAPARGTRPRGCATGSR |
| | PRDCLDVLLSGQQDDGVYSVFPTHYPAGFQVYCDMRTDGGGWTVFQRREDGSVNFFRGWD |
| | AYRDGFGRITGEHWLGLKRIHALTTQAAYELHVDLEDFENGTAYARYGSFGVGLESVDPE |
| | EDGYPLTVADYSGTAGDSLLKHSGMRFTTKDRDSDHSENNCAAFYRGAWWYRNCHTSNLN |
| | GQYLRGAHASYADGVEWSSWTGWQYSLKFSEMKIRPVREDR |
| 81544 | GDPD5 (glycerophosphodiester phosphodiesterase domain; SEQ ID NO: |
| Uniprot | 15) |
| Q8WTR4 | MVRHQPLQYYEPQLCLSCLTGIYGCRWKRYQRSHDDTTPWERLWFLLLTFTFGLTLTWLY |
| | FWWEVHNDYDEFNWYLYNRMGYWSDWPVPILVTTAAAFAYIAGLLVLALCHIAVGQQMNL |
| | HWLHKIGLVVILASTVVAMSAVAQLWEDEWEVLLISLQGTAPFLHVGAVAAVTMLSWIVA |
| | GQFARAERTSSQVTILCTFFTVVFALYLAPLTISSPCIMEKKDLGPKPALIGHRGAPMLA |
| | PEHTLMSFRKALEQKLYGLQADITISLDGVPFLMHDTTLRRTINVEEEFPELARRPASML |
| | NWTTLQRLNAGQWELKTDPFWTASSLSPSDHREAQNQSICSLAELLELAKGNATLLLNLR |
| | DPPREHPYRSSFINVTLEAVLHSGFPQHQVMWLPSRQRPLVRKVAPGFQQTSGSKEAVAS |
| | LRRGHIQRLNLRYTQVSRQELRDYASWNLSVNLYTVNAPWLESLLWCAGVPSVTSDNSHA |
| | LSQVPSPLWIMPPDEYCLMWVTADLVSFTLIVGIFVLQKWRLGGIRSYNPEQIMLSAAVR |
| | RTSRDVSIMKEKLIFSEISDGVEVSDVLSVCSDNSYDTYANSTATPVGPRGGGSHTKTLI |
| | ERSGR |
| 3855 | KRT7 (keratin 7; SEQ ID NO: 16) |
| Uniprot | MSIHFSSPVFTSRSAAFSGRGAQVRESSARPGGLGSSSLYGLGASRPRVAVRSAYGGPVG |
| P08729 | AGIREVTINQSLLAPLRLDADPSLQRVRQEESEQIKTINNKFASFIDKVRFLEQQNKLLE |
| | TKWILLQEQKSAKSSRLPDIFEAQIAGLRGQLEALQVDGGRLEAELRSMQDVVEDEKNKY |
| | EDEINHRTAAENEFVVLKKDVDAAYMSKVELEAKVDALNDEINFLRTLNETELTELQSQI |
| | SDTSVVLSMDNSRSLDLDGIIAEVKAQYEEMAKCSRAEAEAWYQTKFETLQAQAGKHGDD |
| | LRNTRNEISEMNRAIQRLQAEIDNIKNQRAKLEAAIAEAEERGELALKDARAKQEELEAA |
| | LQRGKQDMARQLREYQELMSVKLALDIEIATYRKLLEGEESRLAGDGVGAVNISVMNSTG |
| | GSSSGGGIGLILGGTMGSNALSFSSSAGPGLLKAYSIRTASASRRSARD |
| 3985 | LIMK2 (LIM domain kinase 2; SEQ ID NO: 17) |
| Uniprot | MSALAGEDVWRCPGCGDHIAPSQIWYRTVNETWHGSCFRCSECQDSLINWYYEKDGKLYC |
| P53671 | PKDYWGKFGEFCHGCSLLMTGPFMVAGEFKYHPECFACMSCKVIIEDGDAYALVQHATLY |
| | CGKCHNEVVLAPMFERLSTESVQEQLPYSVTLISMPATTEGRRGFSVSVESACSNYATTV |
| | QVKEVNRMHISPNNRNAIHPGDRILEINGTPVRTLRVEEVEDAISQTSQTLQLLIEHDPV |
| | SQRLDQLRLEARLAPHMQNAGHPHALSTLDTKENLEGTLRRRSLRRSNSISKSPGPSSPK |
| | EPLLESRDISRSESLRCSSSYSQQIFRPCDLIHGEVLGKGFFGQAIKVTHKATGKVMVMK |
| | ELIRCDEETQKTFLTEVKVMRSLDHPNVLKFIGVLYKDKKLNLLTEYIEGGTLKDFLRSM |
| | DPFPWQQKVRFAKGIASGMAYLHSMCIIHRDLNSHNCLIKLDKTVVVADFGLSRLIVEER |
| | KRAPMEKATTKKRTLRKNDRKKRYTVVGNPYWMAPEMLNGKSYDETVDIFSFGIVLCEII |
| | GQVYADPDCLPRTLDEGLNVKLEWEKFVPTDCPPAFFPLAAICCRLEPESRPAFSKLEDS |
| | FEALSLYLGELGIPLPAELEELDHTVSMQYGLTRDSPP |
| 114783 | LMTK3 (lemur tyrosine kinase 3; SEQ ID NO: 18) |
| Uniprot | MPAPGALILLAAVSASGCLASPAHPDGFALGRAPLAPPYAVVLISCSGLLAFIFLLLTCL |
| Q96Q04 | CCKRGDVGFKEFENPEGEDCSGEYTPPAEETSSSQSLPDVYILPLAEVSLPMPAPQPSHS |
| | DMTTPLGLSRQHLSYLQEIGSGWEGKVILGEIFSDYTPAQVVVKELRASAGPLEQRKFIS |
| | EAQPYRSLQHPNVLQCLGLCVETLPFLLIMEFCQLGDLKRYLRAQRPPEGLSPELPPRDL |
| | RTLQRMGLEIARGLAHLASHNYVHSDLALRNCLLTSDLTVRIGDYGLAHSNYKEDYYLTP |
| | ERLWIPLRWAAPELLGELHGTEMVVDQSRESNIWSLGVTLWELFEFGAQPYRHLSDEEVL |
| | AFVVRQQHVKLARPRIKLPYADYWYDILQSCWRPPAQRPSASDLQLQLTYLLSERPPRPP |
| | PPPPPPRDGPFPWPWPPAHSAPRPGTLSSPFPLLDGFPGADPDDVLTVTESSRGLNLECL |
| | WEKARRGAGRGGGAPAWQPASAPPAPHANPSNPFYEALSTPSVLPVISARSPSVSSEYYI |
| | RLEEHGSPPEPLFPNDWDPLDPGVPAPQAPQAPSEVPQLVSETWASPLFPAPRPFPAQSS |
| | ASGSFLLSGWDPEGRGAGETLAGDPAEVLGERGTAPWVEEEEEEEEGSSPGEDSSSLGGG |
| | PSRRGPLPCPLCSREGACSCLPLERGDAVAGWGGHPALGCPHPPEDDSSLRAERGSLADL |
| | PMAPPASAPPEFLDPLMGAAAPQYPGRGPPPAPPPPPPPPRAPADPAASPDPPSAVASPG |
| | SGLSSPGPKPGDSGYETETPFSPEGAFPGGGAAEEEGVPRPRAPPEPPDPGAPRPPPDPG |
| | PLPLPGPREKPTFVVQVSTEQLLMSLREDVTRNLLGEKGATARETGPRKAGRGPGNREKV |
| | PGLNRDPTVLGNGKQAPSLSLPVNGVTVLENGDQRAPGIEEKAAENGALGSPEREEKVLE |
| | NGELTPPRREEKALENGELRSPEAGEKVLVNGGLIPPKSEDKVSENGGLREPRNTERPPE |
| | TGPWRAPGPWEKTPESWGPAPTIGEPAPETSLERAPAPSAVVSSRNGGETAPGPLGPAPK |
| | NGTLEPGTERRAPETGGAPRAPGAGRLDLGSGGRAPVGTGTAPGGGPGSGVDAKAGWVDN |
| | TRPQPPPPPLPPPPEAQPRRLEPAPPRARPEVAPEGEPGAPDSRAGGDTALSGDGDPPKP |
| | ERKGPEMPRLFLDLGPPQGNSEQIKARLSRLSLALPPLTLTPFPGPGPRRPPWEGADAGA |
| | AGGEAGGAGAPGPAEEDGEDEDEDEEEDEEAAAPGAAAGPRGPGRARAAPVPVVVSSADA |
| | DAARPLRGLLKSPRGADEPEDSELERKRKMVSFHGDVTVYLEDQETPTNELSVQAPPEGD |
| | TDPSTPPAPPTPPHPATPGDGEPSNDSGEGGSFEWAEDFPLLPPPGPPLCFSRFSVSPAL |
| | ETPGPPARAPDARPAGPVEN |
| 4016 | LOXL1 (lysyl oxidase like 1; SEQ ID NO: 19) |
| Uniprot | MALARGSRQLGALVWGACLCVLVHGQQAQPGQGSDPARWRQLIQWENNGQVYSLLNSGSE |
| Q08397 | YVPAGPQRSESSSRVLLAGAPQAQQRRSHGSPRRRQAPSLPLPGRVGSDTVRGQARHPFG |
| | FGQVPDNWREVAVGDSTGMARARTSVSQQRHGGSASSVSASAFASTYRQQPSYPQQFPYP |
| | QAPFVSQYENYDPASRTYDQGFVYYRPAGGGVGAGAAAVASAGVIYPYQPRARYEEYGGG |
| | EELPEYPPQGFYPAPERPYVPPPPPPPDGLDRRYSHSLYSEGTPGFEQAYPDPGPEAAQA |
| | HGGDPRLGWYPPYANPPPEAYGPPRALEPPYLPVRSSDTPPPGGERNGAQQGRLSVGSVY |
| | RPNQNGRGLPDLVPDPNYVQASTYVQRAHLYSLRCAAEEKCLASTAYAPEATDYDVRVLL |
| | RFPQRVKNQGTADFLPNRPRHTWEWHSCHQHYHSMDEFSHYDLLDAATGKKVAEGHKASF |
| | CLEDSTCDEGNLKRYACTSHTQGLSPGCYDTYNADIDCQWIDITDVQPGNYILKVHVNPK |
| | YIVLESDETNNVVRCNIHYTGRYVSATNCKIVQS |
| 4430 | MYO1B (myosin IB; SEQ ID NO: 20) |
| Uniprot | MAKMEVKTSLLDNMIGVGDMVLLEPLNEETFINNLKKRFDHSEIYTYIGSVVISVNPYRS |
| O43795 | LPIYSPEKVEEYRNRNFYELSPHIFALSDEAYRSLRDQDKDQCILITGESGAGKTEASKL |
| | VMSYVAAVCGKGAEVNQVKEQLLQSNPVLEAFGNAKTVRNDNSSREGKYMDIEFDFKGDP |
| | LGGVISNYLLEKSRVVKQPRGERNFHVFYQLLSGASEELLNKLKLERDESRYNYLSLDSA |
| | KVNGVDDAANFRTVRNAMQIVGFMDHEAESVLAVVAAVLKLGNIEFKPESRVNGLDESKI |
| | KDKNELKEICELTGIDQSVLERAFSFRTVEAKQEKVSTTLNVAQAYYARDALAKNLYSRL |
| | FSWLVNRINESIKAQTKVRKKVMGVLDIYGFEIFEDNSFEQFIINYCNEKLQQIFIELTL |
| | KEEQEEYIREDIEWTHIDYENNAIICDLIENNINGILAMLDEECLRPGTVTDETFLEKLN |
| | QVCATHQHFESRMSKCSRFINDTSLPHSCFRIQHYAGKVLYQVEGFVDKNNDLLYRDESQ |
| | AMWKASHALIKSIFPEGNPAKINLKRPPTAGSQFKASVATLMKNLQTKNPNYIRCIKPND |
| | KKAAHIFNEALVCHQIRYLGLLENVRVRRAGYAFRQAYEPCLERYKMLCKQTWPHWKGPA |
| | RSGVEVLFNELEIPVEEYSFGRSKIFIRNPRTLFKLEDLRKQRLEDLATLIQKIYRGWKC |
| | RTHFLLMKKSQIVIAAWYRRYAQQKRYQQTKSSALVIQSYIRGWKARKILRELKHQKRCK |
| | EAVTTIAAYWHGTQARRELRRLKEEARNKHAIAVIWAYWLGSKARRELKRLKEEARRKHA |
| | VAVIWAYWLGLKVRREYRKEFRANAGKKIYEFTLQRIVQKYFLEMKNKMPSLSPIDKNWP |
| | SRPYIFLDSTHKELKRIFHLWRCKKYRDQFTDQQKLIYEEKLEASELFKDKKALYPSSVG |
| | QPFQGAYLEINKNPKYKKLKDAIEEKIIIAEVVNKINRANGKSTSRIFLLTNNNLLLADQ |
| | KSGQIKSEVPLVDVTKVSMSSQNDGFFAVHLKEGSEAASKGDELESSDHLIEMATKLYRT |
| | TLSQTKQKLNIEISDEFLVQFRQDKVCVKFIQGNQKNGSVPTCKRKNNRLLEVAVP |
| 5097 | PCDH1 (protocadherin 1; SEQ ID NO: 21) |
| Uniprot | MDSGAGGRRCPEAALLILGPPRMEHLRHSPGPGGQRLLLPSMLLALLLLLAPSPGHATRV |
| Q08174 | VYKVPEEQPPNTLIGSLAADYGFPDVGHLYKLEVGAPYLRVDGKTGDIFTTETSIDREGL |
| | RECQNQLPGDPCILEFEVSITDLVQNGSPRLLEGQIEVQDINDNTPNFASPVITLAIPEN |
| | TNIGSLFPIPLASDRDAGPNGVASYELQAGPEAQELFGLQVAEDQEEKQPQLIVMGNLDR |
| | ERWDSYDLTIKVQDGGSPPRASSALLRVTVLDTNDNAPKFERPSYEAELSENSPIGHSVI |
| | QVKANDSDQGANAEIEYTFHQAPEVVRRLLRLDRNTGLITVQGPVDREDLSTLRESVLAK |
| | DRGTNPKSARAQVVVTVKDMNDNAPTIEIRGIGLVTHQDGMANISEDVAEETAVALVQVS |
| | DRDEGENAAVTCVVAGDVPFQLRQASETGSDSKKKYFLQTTTPLDYEKVKDYTIEIVAVD |
| | SGNPPLSSTNSLKVQVVDVNDNAPVFTQSVTEVAFPENNKPGEVIAEITASDADSGSNAE |
| | LVYSLEPEPAAKGLFTISPETGEIQVKTSLDREQRESYELKVVAADRGSPSLQGTATVLV |
| | NVLDCNDNDPKFMLSGYNFSVMENMPALSPVGMVTVIDGDKGENAQVQLSVEQDNGDEVI |
| | QNGTGTILSSLSFDREQQSTYTFQLKAVDGGVPPRSAYVGVTINVLDENDNAPYITAPSN |
| | TSHKLLTPQTRLGETVSQVAAEDFDSGVNAELIYSIAGGNPYGLFQIGSHSGAITLEKEI |
| | ERRHHGLHRLVVKVSDRGKPPRYGTALVHLYVNETLANRTLLETLLGHSLDTPLDIDIAG |
| | DPEYERSKQRGNILFGVVAGVVAVALLIALAVLVRYCRQREAKSGYQAGKKETKDLYAPK |
| | PSGKASKGNKSKGKKSKSPKPVKPVEDEDEAGLQKSLKENLMSDAPGDSPRIHLPLNYPP |
| | GSPDLGRHYRSNSPLPSIQLQPQSPSASKKHQVVQDLPPANTFVGTGDTTSTGSEQYSDY |
| | SYRTNPPKYPSKQVGQPFQLSTPQPLPHPYHGAIWTEVWE |
| 9124 | PDLIM1 (PDZ and LIM domain 1; SEQ ID NO: 22) |
| Uniprot | MTTQQIDLQGPGPWGFRLVGGKDFEQPLAISRVTPGSKAALANLCIGDVITAIDGENTSN |
| O00151 | MTHLEAQNRIKGCTDNLTLTVARSEHKVWSPLVTEEGKRHPYKMNLASEPQEVLHIGSAH |
| | NRSAMPFTASPASSTTARVITNQYNNPAGLYSSENISNENNALESKTAASGVEANSRPLD |
| | HAQPPSSLVIDKESEVYKMLQEKQELNEPPKQSTSELVLQEILESEEKGDPNKPSGERSV |
| | KAPVTKVAASIGNAQKLPMCDKCGTGIVGVFVKLRDRHRHPECYVCTDCGTNLKQKGHFF |
| | VEDQIYCEKHARERVTPPEGYEVVTVEPK |
| 8398 | PLA2G6 (phospholipase A2 group VI; SEQ ID NO: 23) |
| Uniprot | MQFFGRLVNTFSGVTNLFSNPFRVKEVAVADYTSSDRVREEGQLILFQNTPNRTWDCVLV |
| O60733 | NPRNSQSGFRLFQLELEADALVNFHQYSSQLLPFYESSPQVLHTEVLQHLTDLIRNHPSW |
| | SVAHLAVELGIRECFHHSRIISCANCAENEEGCTPLHLACRKGDGEILVELVQYCHTQMD |
| | VTDYKGETVFHYAVQGDNSQVLQLLGRNAVAGLNQVNNQGLTPLHLACQLGKQEMVRVLL |
| | LCNARCNIMGPNGYPIHSAMKFSQKGCAEMIISMDSSQIHSKDPRYGASPLHWAKNAEMA |
| | RMLLKRGCNVNSTSSAGNTALHVAVMRNREDCAIVLLTHGANADARGEHGNTPLHLAMSK |
| | DNVEMIKALIVFGAEVDTPNDFGETPTFLASKIGRLVTRKAILTLLRTVGAEYCFPPIHG |
| | VPAEQGSAAPHHPFSLERAQPPPISLNNLELQDLMHISRARKPAFILGSMRDEKRTHDHL |
| | LCLDGGGVKGLIIIQLLIAIEKASGVATKDLFDWVAGTSTGGILALAILHSKSMAYMRGM |
| | YFRMKDEVERGSRPYESGPLEEFLKREFGEHTKMTDVRKPKVMLTGTLSDRQPAELHLER |
| | NYDAPETVREPRENQNVNLRPPAQPSDQLVWRAARSSGAAPTYFRPNGRELDGGLLANNP |
| | TLDAMTEIHEYNQDLIRKGQANKVKKLSIVVSLGTGRSPQVPVTCVDVERPSNPWELAKT |
| | VFGAKELGKMVVDCCTDPDGRAVDRARAWCEMVGIQYFRLNPQLGTDIMLDEVSDTVLVN |
| | ALWETEVYIYEHREEFQKLIQLLLSP |
| 5875 | RABGGTA (Rab geranylgeranyltransferase subunit alpha; SEQ ID NO: |
| Uniprot | 24) |
| Q92696 | MHGRLKVKTSEEQAEAKRLEREQKLKLYQSATQAVFQKRQAGELDESVLELTSQILGANP |
| | DEATLWNCRREVLQQLETQKSPEELAALVKAELGFLESCLRVNPKSYGTWHHRCWLLGRL |
| | PEPNWTRELELCARFLEVDERNFHCWDYRRFVATQAAVPPAEELAFTDSLITRNESNYSS |
| | WHYRSCLLPQLHPQPDSGPQGRLPEDVLLKELELVQNAFFTDPNDQSAWFYHRWLLGRAD |
| | PQDALRCLHVSRDEACLTVSFSRPLLVGSRMEILLLMVDDSPLIVEWRTPDGRNRPSHVW |
| | LCDLPAASLNDQLPQHTFRVIWTAGDVQKECVLLKGRQEGWCRDSTTDEQLFRCELSVEK |
| | STVLQSELESCKELQELEPENKWCLLTIILLMRALDPLLYEKETLQYFQTLKAVDPMRAT |
| | YLDDIRSKELLENSVLKMEYAEVRVLHLAHKDLTVLCHLEQLLLVTHLDLSHNRLRTLPP |
| | ALAALRCLEVLQASDNAIESLDGVTNLPRLQELLLCNNRLQQPAVLQPLASCPRLVLLNL |
| | QGNPLCQAVGILEQLAELLPSVSSVLT |
| 6337 | SCNN1A (sodium channel epithelial 1 subunit alpha; SEQ ID NO: 25) |
| Uniprot | MEGNKLEEQDSSPPQSTPGLMKGNKREEQGLGPEPAAPQQPTAEEEALIEFHRSYRELFE |
| P37088 | FFCNNTTIHGAIRLVCSQHNRMKTAFWAVLWLCTFGMMYWQFGLLFGEYFSYPVSLNINL |
| | NSDKLVFPAVTICTLNPYRYPEIKEELEELDRITEQTLEDLYKYSSFTTIVAGSRSRRDL |
| | RGTLPHPLQRLRVPPPPHGARRARSVASSLRDNNPQVDWKDWKIGFQLCNQNKSDCFYQT |
| | YSSGVDAVREWYRFHYINILSRLPETLPSLEEDTLGNFIFACRENQVSCNQANYSHEHHP |
| | MYGNCYTENDKNNSNLWMSSMPGINNGLSLMLRAEQNDFIPLLSTVTGARVMVHGQDEPA |
| | FMDDGGFNLRPGVETSISMRKETLDRLGGDYGDCTKNGSDVPVENLYPSKYTQQVCIHSC |
| | FQESMIKECGCAYIFYPRPQNVEYCDYRKHSSWGYCYYKLQVDFSSDHLGCFTKCRKPCS |
| | VTSYQLSAGYSRWPSVTSQEWVFQMLSRQNNYTVNNKRNGVAKVNIFFKELNYKINSESP |
| | SVTMVTLLSNLGSQWSLWFGSSVLSVVEMAELVEDLLVIMFLMLLRRERSRYWSPGRGGR |
| | GAQEVASTLASSPPSHFCPHPMSLSLSQPGPAPSPALTAPPPAYATLGPRPSPGGSAGAS |
| | SSTCPLGGP |
| 92799 | SHKBP1 (SH3KBP1 binding protein 1; SEQ ID NO: 26) |
| Uniprot | MAAAATAAEGVPSRGPPGEVIHLNVGGKRFSTSRQTLTWIPDSFFSSLLSGRISTLKDET |
| Q8TBC3 | GAIFIDRDPTVFAPILNFLRTKELDPRGVHGSSLLHEAQFYGLTPLVRRLQLREELDRSS |
| | CGNVLFNGYLPPPVFPVKRRNRHSLVGPQQLGGRPAPVRRSNTMPPNLGNAGLLGRMLDE |
| | KTPPSPSGQPEEPGMVRLVCGHHNWIAVAYTQFLVCYRLKEASGWQLVESSPRLDWPIER |
| | LALTARVHGGALGEHDKMVAAATGSEILLWALQAEGGGSEIGVFHLGVPVEALFFVGNQL |
| | IATSHTGRIGVWNAVTKHWQVQEVQPITSYDAAGSFLLLGCNNGSIYYVDVQKFPLRMKD |
| | NDLLVSELYRDPAEDGVTALSVYLTPKTSDSGNWIEIAYGTSSGGVRVIVQHPETVGSGP |
| | QLFQTFTVHRSPVTKIMLSEKHLISVCADNNHVRTWSVTRFRGMISTQPGSTPLASFKIL |
| | ALESADGHGGCSAGNDIGPYGERDDQQVFIQKVVPSASQLFVRLSSTGQRVCSVRSVDGS |
| | PTTAFTVLECEGSRRLGSRPRRYLLTGQANGSLAMWDLTTAMDGLGQAPAGGLTEQELME |
| | QLEHCELAPPAPSAPSWGCLPSPSPRISLTSLHSASSNTSLSGHRGSPSPPQAEARRRGG |
| | GSFVERCQELVRSGPDLRRPPTPAPWPSSGLGTPLTPPKMKLNETSF |
| 55315 | SLC29A3 (solute carrier family 29 member 3; SEQ ID NO: 27) |
| Uniprot | MAVVSEDDFQHSSNSTYRTTSSSLRADQEALLEKLLDRPPPGLQRPEDRECGTYIIFFSL |
| Q9BZD2 | GIGSLLPWNFFITAKEYWMFKLRNSSSPATGEDPEGSDILNYFESYLAVASTVPSMLCLV |
| | ANFLLVNRVAVHIRVLASLTVILAIFMVITALVKVDTSSWTRGFFAVTIVCMVILSGAST |
| | VFSSSIYGMTGSFPMRNSQALISGGAMGGTVSAVASLVDLAASSDVRNSALAFFLTATVE |
| | LVLCMGLYLLLSRLEYARYYMRPVLAAHVESGEEELPQDSLSAPSVASRFIDSHTPPLRP |
| | ILKKTASLGFCVTYVFFITSLIYPAICTNIESLNKGSGSLWTTKFFIPLTTFLLYNFADL |
| | CGRQLTAWIQVPGPNSKALPGFVLLRTCLIPLFVLCNYQPRVHLKTVVFQSDVYPALLSS |
| | LLGLSNGYLSTLALLYGPKIVPRELAEATGVVMSFYVCLGLTLGSACSTLLVHLI |
| 56848 | SPHK2 (sphingosine kinase 2; SEQ ID NO: 28) |
| Uniprot | MNGHLEAEEQQDQRPDQELTGSWGHGPRSTLVRAKAMAPPPPPLAASTPLLHGEFGSYPA |
| Q9NRA0 | RGPRFALTLTSQALHIQRLRPKPEARPRGGLVPLAEVSGCCTLRSRSPSDSAAYFCIYTY |
| | PRGRRGARRRATRTFRADGAATYEENRAEAQRWATALTCLLRGLPLPGDGEITPDLLPRP |
| | PRLLLLVNPFGGRGLAWQWCKNHVLPMISEAGLSFNLIQTERQNHARELVQGLSLSEWDG |
| | IVTVSGDGLLHEVINGLLDRPDWEEAVKMPVGILPCGSGNALAGAVNQHGGFEPALGLDL |
| | LINCSLLLCRGGGHPLDLLSVTLASGSRCFSFLSVAWGFVSDVDIQSERFRALGSARFTL |
| | GTVLGLATLHTYRGRLSYLPATVEPASPTPAHSIPRAKSELTLTPDPAPPMAHSPLHRSV |
| | SDLPLPLPQPALASPGSPEPLPILSINGGGPELAGDWGGAGDAPLSPDPLLSSPPGSPKA |
| | ALHSPVSEGAPVIPPSSGLPLPTPDARVGASTCGPPDHLLPPIGTPLPPDWVTLEGDEVL |
| | MLAISPSHLGADLVAAPHARFDDGLVHLCWVRSGISRAALLRLFLAMERGSHFSLGCPQL |
| | GYAAARAFRLEPLTPRGVLTVDGEQVEYGPLQAQMHPGIGTLLTGPPGCPGREP |
| 79816 | TLE6 (TLE family member 6, subcortical maternal complex member; |
| Uniprot | SEQ ID NO: 29) |
| Q9H808 | MTSRDQPRPKGPPKSTSPCPGISNSESSPTLNYQGILNRLKQFPRESPHFAAELESIYYS |
| | LHKIQQDVAEHHKQIGNVLQIVESCSQLQGFQSEEVSPAEPASPGTPQQVKDKTLQESSF |
| | EDIMATRSSDWLRRPLGEDNQPETQLFWDKEPWFWHDTLTEQLWRIFAGVHDEKAKPRDR |
| | QQAPGLGQESKAPGSCDPGTDPCPEDASTPRPPEASSSPPEGSQDRNTSWGVVQEPPGRA |
| | SRFLQSISWDPEDFEDAWKRPDALPGQSKRLAVPCKLEKMRILAHGELVLATAISSFTRH |
| | VFTCGRRGIKVWSLTGQVAEDRFPESHLPIQTPGAFLRTCLLSSNSRSLLTGGYNLASVS |
| | VWDLAAPSLHVKEQLPCAGLNCQALDANLDANLAFASFTSGVVRIWDLRDQSVVRDLKGY |
| | PDGVKSIVVKGYNIWTGGPDACLRCWDQRTIMKPLEYQFKSQIMSLSHSPQEDWVLLGMA |
| | NGQQWLQSTSGSQRHMVGQKDSVILSVKFSPFGQWWASVGMDDFLGVYSMPAGTKVFEVP |
| | EMS PVTCCDVSSNNRLVVTGSGEHASVYQITY |
| 79639 | TMEM53 (transmembrane protein 53; SEQ ID NO: 30) |
| Uniprot | MASAELDYTIEIPDQPCWSQKNSPSPGGKEAETRQPVVILLGWGGCKDKNLAKYSAIYKK |
| Q6P2H8 | RGCIVIRYTAPWHMVFFSESLGIPSLRVLAQKLLELLEDYEIEKEPLLFHVFSNGGVMLY |
| | RYVLELLQTRRECRLRVVGTIFDSAPGDSNLVGALRALAAILERRAAMLRLLLLVAFALV |
| | VVLFHVLLAPITALFHTHEYDRLQDAGSRWPELYLYSRADEVVLARDIERMVEARLARRV |
| | LARSVDFVSSAHVSHLRDYPTYYTSLCVDFMRNCVRC |
| 641649 | TMEM91 (transmembrane protein 91; SEQ ID NO: 31) |
| Uniprot | MDSPSLRELQQPLLEGTECETPAQKPGRHELGSPLREIAFAESIRGLQFLSPPLPSVSAG |
| Q6ZNR0 | LGEPRPPDVEDMSSSDSDSDWDGGSRLSPFLPHDHLGLAVFSMLCCFWPVGIAAFCLAQK |
| | TNKAWAKGDIQGAGAASRRAFLLGVLAVGLGVCTYAAALVTLAAYLASRDPP |

### Histone Deacetylase 1 (HDAC1) Protein

HDAC1 catalyzes the deacetylation of lysine residues on the N-terminal part of the core histones (H2A, H2B, H3 and H4). The HDAC1 gene is located on chromosome 1 (Gene ID: 3065; location NC_000001.11 (32292083 . . . 32333626). An example of an amino acid sequence for HDAC1 isoform 1 is available as UNIPROT accession no. Q13547 and shown below as SEQ ID NO:32. MAQTQGTRRKVCYYYDGDVGNYYYGQGHPMKPHRIRMTHNLLLNYGLYRKMEIYRPHKANAE EMTKYHSDDYIKFLRSIRPDNMSEYSKQMQRFNVGEDCPVFDGLFEFCQLSTGGSVASAVKL NKQQTDIAVNWAGGLHHAKKSEASGFCYVNDIVLAILELLKYHQRVLYIDIDIHHGDGVEEA FYTTDRVMTVSFHKYGEYFPGTGDLRDIGAGKGKYYAVNYPLRDGIDDESYEAI FKPVMSKV MEMFQPSAVVLQCGSDSLSGDRLGCFNLTIKGHAKCVEFVKSFNLPMLMLGGGGYTIRNVAR CWTYETAVALDTEIPNELPYNDYFEYFGPDFKLHISPSNMTNQNTNEYLEKIKQRLFENLRM LPHAPGVQMQAIPEDAIPEESGDEDEDDPDKRISICSSDKRIACEEEFSDSEEEGEGGRKNS SNFKKAKRVKTEDEKEKDPEEKKEVTEEEKTKEEKPEAKGVKEEVKLA

A cDNA sequence encoding the SEQ ID NO:32 HDAC1 protein is available as NCBI accession no. NM 004964.3, shown below as SEQ ID NO:33:

| | |
|---|---|
| 1 | cccctccccc ctgggtcgga cgctgagcgg agccgcgggc gggagggcgg acggaccgac |
| 61 | tgacggtagg gacgggaggc gagcaagatg gcgcagacgc agggcacccg gaggaaagtc |
| 121 | tgttactact acgacgggga tgttggaaat tactattatg gacaaggcca cccaatgaag |
| 181 | cctcaccgaa tccgcatgac tcataatttg ctgctcaact atggtctcta ccgaaaaatg |
| 241 | gaaatctatc gccctcacaa agccaatgct gaggagatga ccaagtacca cagcgatgac |
| 301 | tacattaaat tcttgcgctc catccgtcca gataacatgt cggagtacag caagcagatg |
| 361 | cagagattca acgttggtga ggactgtcca gtattcgatg gcctgtttga gttctgtcag |
| 421 | ttgtctactg gtcgttctgt ggcaagtgct gtgaaactta ataagcagca gacggacatc |
| 481 | gctgtgaatt gggctggggg cctgcaccat gcaaagaagt ccgaggcatc tggcttctgt |
| 541 | tacgtcaatg atatcgtctt ggccatcctg gaactgctaa agtatcacca gagggtgctg |
| 601 | tacattgaca ttgatattca ccatggtgac ggcgtggaag aggccttcta caccacggac |
| 661 | cgggtcatga ctgtgtcctt tcataagtat ggagagtact tcccaggaac tggggaccta |
| 721 | cgggatatcg gggctggcaa aggcaagtat tatgctgtta actacccgct ccgagacggg |
| 781 | attgatgacg agtcctatga ggccattttc aagccggtca tgtccaaagt aatggagatg |
| 841 | ttccagccta gtgcggtggt cttacagtgt ggctcagact ccctatctgg ggatcggtta |
| 901 | ggttgcttca atctaactat caaaggacac gccaagtgtg tggaatttgt caagagcttt |
| 961 | aacctgccta tgctgatgct gggaggcggt ggttacacca ttcgtaacgt tgcccggtgc |
| 1021 | tggacatatg agacagctgt ggccctggat acggagatcc ctaatgagct tccatacaat |
| 1081 | gactactttg aatactttgg accagatttc aagctccaca tcagtccttc caatatgact |
| 1141 | aaccagaaca cgaatgagta cctggagaag atcaaacagc gactgtttga gaaccttaga |
| 1201 | atgctgccgc acgcacctgg ggtccaaatg caggcgattc ctgaggacgc catccctgag |
| 1261 | gagagtggcg atgaggacga agacgaccct gacaagcgca tctcgatctg ctcctctgac |
| 1321 | aaacgaattg cctgtgagga agagttctcc gattctgaag aggagggaga ggggggccgc |
| 1381 | aagaactctt ccaacttcaa aaaagccaag agagtcaaaa cagaggatga aaaagagaaa |
| 1441 | gacccagagg agaagaaaga agtcaccgaa gaggagaaaa ccaaggagga gaagccagaa |
| 1501 | gccaaagggg tcaaggagga ggtcaagttg gcctgaatgg acctctccag ctctggcttc |
| 1561 | ctgctgagtc cctcacgttt cttccccaac ccctcagatt ttatattttc tatttctctg |
| 1621 | tgtatttata taaaaattta ttaaatataa atatccccag ggacagaaac caaggccccg |
| 1681 | agctcagggc agctgtgctg ggtgagctct tccaggagcc accttgccac ccattcttcc |
| 1741 | cgttcttaac tttgaaccat aaagggtgcc aggtctgggt gaaagggata cttttatgca |
| 1801 | accataagac aaactcctga aatgccaagt gcctgcttag tagctttgga aaggtgccct |
| 1861 | tattgaacat tctagaaggg gtggctgggt cttcaaggat ctcctgtttt tttcaggctc |
| 1921 | ctaaagtaac atcagccatt tttagattgg ttctgttttc gtaccttccc actggcctca |
| 1981 | agtgagccaa gaaacactgc ctgccctctg tctgtcttct cctaattctg caggtggagg |
| 2041 | ttgctagtct agtttccttt ttgagatact attttcattt ttgtgagcct ctttgtaata |
| 2101 | aaatggtaca tttctata |

### Histone Deacetylase 7 (HDAC7)

HDAC7 catalyzes the deacetylation of lysine residues on the N-terminal part of the core histones (H2A, H2B, H3 and H4). The HDAC7 gene is located on chromosome 12 (Gene ID: 51564; location NC_000012.12 (47782722..47821344). An example of an amino acid sequence for HDAC7 is available as UNIPROT accession no. Q8WUI4 and shown below as SEQ ID NO:34.

MDLRVGQRPPVEPPPEPTLLALQRPQRLHHHLFLAGLQQQRSVEPMRLSMDTPMPELQVG
PQEQELRQLLHKDKSKRSAVASSVVKQKLAEVILKKQQAALERTVHPNSPGIPYRTLEPL
ETEGATRSMLSSFLPPVPSLPSDPPEHFPLRKTVSEPNLKLRYKPKKSLERRKNPLLRKE
SAPPSLRRRPAETLGDSSPSSSSTPASGCSSPNDSEHGPNPILGSEALLGQRLRLQETSV
APFALPTVSLLPAITLGLPAPARADSDRRTHPTLGPRGPILGSPHTPLFLPHGLEPEAGG
TLPSRLQPILLLDPSGSHAPLLTVPGLGPLPFHFAQSLMTTERLSGSGLHWPLSRTRSEP
LPPSATAPPPPGPMQPRLEQLKTHVQVIKRSAKPSEKPRLRQIPSAEDLETDGGGPGQVV
DDGLEHRELGHGQPEARGPAPLQQHPQVLLWEQQRLAGRLPRGSTGDTVLLPLAQGGHRP
LSRAQSSPAAPASLSAPEPASQARVLSSSETPARTLPFTTGLIYDSVMLKHQCSCGDNSR
HPEHAGRIQSIWSRLQERGLRSQCECLRGRKASLEELQSVHSERHVLLYGTNPLSRLKLD
NGKLAGLLAQRMFVMLPCGGVGVDTDTIWNELHSSNAARWAAGSVTDLAFKVASRELKNG
EAVVRPPGHHADHSTAMGECFENSVAIACRQLQQQSKASKILIVDWDVHHGNGTQQTEYQ
DPSVIYISLHRHDDGNFFPGSGAVDEVGAGSGEGENVNVAWAGGLDPPMGDPEYLAAFRI
VVMPIAREFSPDLVLVSAGFDAAEGHPAPLGGYHVSAKCFGYMTQQLMNLAGGAVVLALE
GGHDLTAICDASEACVAALLGNRVDPLSEEGWKQKPNLNAIRSLEAVIRVHSKYWGCMQR
LASCPDSWVPRVPGADKEEVEAVTALASLSVGILAEDRPSEQLVEEEEPMNL

A cDNA sequence encoding the amino acid sequence of SEQ ID NO:34 HDAC1 protein is available as NCBI accession no. NM_001098416.4, shown below as SEQ ID NO:35:

| | |
|---|---|
| 1 | cttcctctcg ccgtctcaca gtcgctctgc agcctccggc gactgggggg atgtgaggcc |
| 61 | ggcgccccag ccccccgccc cgccatgagc cccccgctct gagggccccg gcccctggat |
| 121 | gcacagcccc ggcgctgatg ggacccaggt gagcccgggt gcccactact gcagccccac |
| 181 | tggcgcaggc tgccccaggc cctgtgcaga cacaccaggc cctcagccgc agcccatgga |
| 241 | cctgcgggtg ggccagcggc ccccagtgga gcccccacca gagcccacat tgctggccct |
| 301 | gcagcgtccc cagcgcctgc accaccacct cttcctagca ggcctgcagc agcagcgctc |
| 361 | ggtggagccc atgaggctct ccatggacac gccgatgccc gagttgcagg tgggacccca |
| 421 | ggaacaagag ctgcggcagc ttctccacaa ggacaagagc aagcgaagtg ctgtagccag |
| 481 | cagcgtggtc aagcagaagc tagcggaggt gattctgaaa aaacagcagg cggccctaga |
| 541 | aagaacagtc catcccaaca gccccggcat tccctacaga accctggagc ccctggagac |
| 601 | ggaaggagcc acccgctcca tgctcagcag ctttttgcct cctgttccca gcctgcccag |
| 661 | tgacccccca gagcacttcc ctctgcgcaa gacagtctct gagcccaacc tgaagctgcg |
| 721 | ctataagccc aagaagtccc tggagcggag gaagaatcca ctgctccgaa aggagagtgc |
| 781 | gccccccagc ctccggCggc ggcccgcaga gaccctcgga gactcctccc caagtagtag |
| 841 | cagcacgccc gcatcagggt gcagctcccc caatgacagc gagcacggcc ccaatcccat |
| 901 | cctgggctcg gaggctgaca gtgaccgcag gacccatccg actctgggcc ctcgggggcc |
| 961 | aatcctgggg agcccccaca ctcccctctt cctgccccat ggcttggagc ccgaggctgg |
| 1021 | gggcaccttg ccctctcgcc tgcagcccat tctcctcctg gacccctcag gctctcatgc |
| 1081 | cccgctgctg actgtgcccg ggcttgggcc cttgcccttc cactttgccc agtccttaat |
| 1141 | gaccaccgag cggctctctg ggtcaggcct ccactggcca ctgagccgga ctcgctcaga |
| 1201 | gcccctgccc cccagtgcca ccgctccccc accgccgggc cccatgcagc cccgcctgga |
| 1261 | gcagctcaaa actcacgtcc aggtgatcaa gaggtcagcc aagccgagtg agaagccccg |
| 1321 | gctgcggcag ataccctcgg ctgaagacct ggagacagat ggcgggggac cgggccaggt |
| 1381 | ggtggacgat ggcctggagc acagggagct gggccatggg cagcctgagg ccagaggccc |
| 1441 | cgctcctctc cagcagcacc ctcaggtgtt gctctgggaa cagcagcgac tggctgggcg |
| 1501 | gctcccccgg ggcagcaccg gggacactgt gctgcttcct ctggcccagg gtgggcaccc |
| 1561 | gcctctgtcc cgggctcagt cttccccagc cgcacctgcc tcactgtcag ccccagagcc |
| 1621 | tgccagccag gcccgagtcc tctccagctc agagacccct gccaggaccc tgcccttcac |
| 1681 | cacagggctg atctatgact cggtcatgct gaagcaccag tgctcctgcg gtgacaacag |
| 1741 | caggcacccg gagcacgccg gccgcatcca gagcatctgg tcccggctgc aggagcgggg |
| 1801 | gctccggagc cagtgtgagt gtctccgagg ccggaaggcc tccctggaag agctgcagtc |
| 1861 | ggtccactct gagcggcacg tgctcctcta cggcaccaac ccgctcagcc gcctcaaact |
| 1921 | ggacaacggg aagctggcag ggctcctggc acagcggatg tttgtgatgc tgccctgtgg |
| 1981 | tggggttggg gtggacactg acaccatctg gaatgagctt cattcctcca atgcagcccg |
| 2041 | ctgggccgct ggcagtgtca ctgacctcgc cttcaaagtg gcttctcgtg agctaaagaa |
| 2101 | tggtttcgct gtggtgcggc ccccaggaca ccatgcagat cattcaacag ccatgggctt |
| 2161 | ctgcttcttc aactcagtgg ccatcgcctg ccggcagctg caacagcaga gcaaggccag |
| 2221 | caagatcctc attgtagact gggacgtgca ccatggcaac ggcacccagc aaaccttcta |
| 2281 | ccaagacccc agtgtgctct acatctccct gcatcgccat gacgacggca acttcttccc |
| 2341 | ggggagtggg gctgtggatg aggtaggggc tggcagcggt gagggcttca atgtcaatgt |
| 2401 | ggcctgggct ggaggtctgg acccccccat gggggatcct gagtacctgg ctgctttcag |
| 2461 | gatagtCgtg atgcccatcg cccgagagtt ctctccagac ctagtcctgg tgtctgctgg |
| 2521 | atttgatgct gctgagggtc acccggcccc actgggtggc taccatgttt ctgccaaatg |
| 2581 | ttttggatac atgacgcagc aactgatgaa cctggcagga ggcgcagtgg tgctggcctt |
| 2641 | ggagggtggc catgacctca cagccatctg tgacgcctct gaggcctgtg tggctgctct |
| 2701 | tctgggtaac agggtggatc ccctttcaga agaaggctgg aaacagaaac ccaacctcaa |
| 2761 | tgccatccgc tctctggagg ccgtgatccg ggtgcacagt aaatactggg gctgcatgca |
| 2821 | gcgcctggcc tcctgtccag actcctgggt gcctagagtg ccaggggctg acaaagaaga |
| 2881 | agtggaggca gtgaccgcac tggcgtccct ctctgtgggc atcctggctg aagataggcc |
| 2941 | ctcggagcag ctggtggagg aggaagaacc tatgaatctc taaggctctg gaaccatctg |
| 3001 | cccgcccacc atgcccttgg gacctggttc tcttctaacc cctggcaata gcccccattc |
| 3061 | ctgggtcttt agagatcctg tgggcaagta gttggaacca gagaacagcc tgcctgcttt |
| 3121 | gacagttatc ccagggagcg tgagaaaatc cctgggtcta gaatgggaac tggagaggac |
| 3181 | cctgagagga gacgggctgg gCggcgaccc ccacagggct ctcgagaaca gattctcccc |
| 3241 | tccagtatgg gccctggctg tggcccccat tcctcaggac tgcacagagg aggactggct |
| 3301 | ccggctccgt cgggctcacc cttaaccact attcctggct ctgcaaaccc cagactttgc |
| 3361 | acacagcctc aggctccaca cagaaatgtg aacttggcct cagacaggct ggcccttcct |
| 3421 | aggctctagg ggctaggggg gagtggggag ccaagaggtc ccatattcct gagtgcaggg |
| 3481 | gtagtccctc tcacctgctt cctcagacga ctctggaagc ttccctctac cactgggcac |
| 3541 | tgagacgaag ctccctgaca gccgagactg gcagccctcc atctggtccg taccctcgcc |
| 3601 | agaggccccc ctacatcaac ctcctggcga tgccctggtg gagcagatgg gtgctctggg |
| 3661 | agtcctgtgc ttcctgatcc aatggtgcca aacccttcat ctccccaaga agcgcagcat |
| 3721 | acccctggga cccctcggcc actgcccact cggggagcct tctctgtttc tggggcctcc |
| 3781 | cccaccatag ctctgattcc caccccacat aggagtagcc tgactgaggg ggaaggggtg |
| 3841 | ggagagaaga tacagacatg gaggagggga ggctgctctg gcaaagtctt caaggctttt |
| 3901 | gggggtccag gcctggggtc aagaaggaaa atgtgtgtga gcatgtgtgt gagtgaggcg |
| 3961 | tgtgtgtgag cgtgtgtgtg agtgaggcgt gtgtgtgtgt ctttcctagg acccaccata |
| 4021 | ccctgtgtat gtatgcatgt ttttgtaaaa aggaagaaaa tggaaaaaaa tctgaacaat |
| 4081 | aaatgtttta tttgctttaa aa |

Isoforms and variants of the ET-29, HDAC1, HDAC7 and ZNF92 genes and gene products can be present in subjects and can be detected, measured, evaluated, and the subjects with such isoforms and variants can be treated by the methods and compositions described herein. Such isoforms and variants can have sequences with between 65-100% sequence identity to a reference sequence, for example with at least at least 65%1, at least 70%, at least 80%, at least 90%, at least 95%, at least 96%, at least 97;% sequence, at least 98%, at least 99%, or at least 99.5% identity to a sequence described herein or a reference sequence (such as one described in the NCBI or Uniprot databases) over a specified comparison window. Optimal alignment may be ascertained or conducted using the homology alignment algorithm of Needleman and Wunsch, J. Mol. Biol. 48:443-53 (1970).

### Definitions

The “absolute amplitude” of correlation expressions means the distance, either positive or negative, from a zero value; i.e., both correlation coefficients −0.35 and 0.35 have an absolute amplitude of 0.35 ET-29, HDAC1, HDAC7 and ZNF92.

“Status” means a state of gene expression of a set of genetic markers whose expression is strongly correlated with a particular phenotype. For example, “ZNF92 status” means a state of gene expression of a set of genetic markers (e.g., HDAC1, HDAC7 markers) whose expression is strongly correlated with that of the ZNF92 gene, wherein the expression pattern of these (e.g. HDAC1, HDAC7,) can differ detectably between tumors expressing the ZNF92 and tumors not expressing ZNF92.

“Good prognosis” means that a patient is expected to have longer overall survival (OS), or progression-free survival (PFS), or disease-specific survival (DSS) or recurrence-free survival (RFS) compared to “poor prognosis” patients. These metrics are typically described by National Cancer Institute (NCI) as overall survival (OS), or progression-free survival (PFS) which is the length of time during and after the treatment of cancer, that a patient lives with the disease but it does not get worse, or disease-specific survival (DSS) that is the percentage of people in a treatment group who have not died from their cancer in a defined period of time, or recurrence-free survival (RFS) that is length of time after primary treatment for a cancer ends that the patient survives without any signs or symptoms of that cancer, also called as disease-free survival (DFS), or relapse-free survival (see website at cancer.gov/publications/dictionaries/cancer-terms/def/rfs).

“Poor prognosis” means that a patient is expected to have a shorter overall survival (OS), or progression-free survival (PFS), or disease-specific survival (DSS) or recurrence-free survival (RFS) compared to “good prognosis” patients.

“Marker” means an entire gene, mRNA, EST, or a protein product derived from that gene, where the expression or level of expression changes under different conditions, where the expression of the gene (or combination of genes) correlates with a certain condition, the gene or combination of genes is a marker for that condition.

“Marker-derived polynucleotides” means the RNA transcribed from a marker gene, any cDNA, or cRNA produced therefrom, and any nucleic acid derived therefrom, such as synthetic nucleic acid having a sequence derived from the gene corresponding to the marker gene.

A “similarity value” is a number that represents the degree of similarity between two things being compared. For example, a similarity value may be a number that indicates the overall similarity between a patient's expression profile using specific phenotype-related markers and a control specific to that phenotype (for instance, the similarity to a “good prognosis” template, where the phenotype is a good prognosis). The similarity value may be expressed as a similarity metric, such as a correlation coefficient, or may simply be expressed as the expression level difference, or the aggregate of the expression level differences, between a patient sample and a template.

The present description is further illustrated by the following examples, which should not be construed as limiting in any way.

### EXAMPLES

### Example 1: Materials and Methods

Reagents: Entinostat (Active motif 14043), Vorinostat (Abcam-ab1444480), Belinostat (Tokyo Chemical Industry-B5888), Niclosamide (Bio-Vision-1826),^{[25-27]} Tanespimycin/17AAG (Bio-Vision-1774) and Pimitespib (Tas1116, Cayman Chemical Company-33779) were reconstituted in DMSO (Sigma Aldrich D-8418). Cell Proliferation Reagent WST-1 (11644807001, Sigma Aldrich).

Cell culture: The ATCC sourced breast cancer cell lines were previously acquired and extensively validated including STR profiling in our previous studies^{[11, 25, 27, 29]}. All the ATCC cell lines including HCC38, HCC202, HCC1187, HCC 1937, HCC 1143, HCC 1954, MFM223, CAL148, MDAMB231, SKBR3, ZR75-1, ZR75-B, MCF7, MDAMB231, MDAMB435, BT549, BT20, and T47D were maintained in their respective media as recommended by ATCC (Gibco, Thermo-Fisher, NY, USA). The inflammatory breast cancer cell lines IBC02 (FC-IBC02) IBC03, KPL4, Sum149, and Sum190 were generously provided by Dr. Sandra Fernandez and cultured in F-12 medium (Gibco 11765-054). All the drug sensitivity experiments were carried out in medium supplemented with 2% heat-inactivated fetal bovine serum. The BPLER and HMLER cells were previously characterized, deposited to the American Type Culture Collection and the European Collection of Authenticated Cell Cultures (ATCC #CRL-3546 and ECACC #20012032 and 20012041) and cultured in BMI-T medium (US Biological, cat #506387.500)^{[27]}. All the cells were cultured at 37° C. and 5% CO₂ in a humidified incubator.

WST cell proliferation assay: The changes in cell proliferation and viability due to treatment with Entinostat, Vorinostat, Belinostat, Niclosamide, Tanespimycin and Pimitespib alone or in combination was determined by WST assay following manufacturer's protocol. Briefly, ˜3000 of cells per well were plated in 96-well plates and treated with various drugs for 7 days. At the end of the specified time following drug treatment, the WST reagent (10 μL) was added into each well, incubated at 37° C. for 2 h and the color absorbance of each well was recorded at 450 nm with a Thermo Labsystems Multiskan Ascent microplate reader with a blank reference.

Drug screening: The Gene Set Cancer Analysis (GSCA) and the Cancer Therapeutics Response Portal (CTRP) online analysis tools were used to identify drugs that may target ZNF92 and its 29 gene signature. These platforms integrate more than 10,000 comprehensive genomic data set of 33 diverse types of cancers from TCGA with 750 drug molecules^{[30-32]}. Graphical representation and Statistical analyses: GraphPad Prism (V9) software was used for statistical analysis and graph generation. All data are expressed as the mean±SD of six independent experiments. The differences between the control and the treatment groups were determined by one-way ANOVA, and significance was determined by using Dunnett's multiple comparison test (P<0.01).

### Example 2: The Intrinsic Properties of the Normal Cell-Origin is Involved in Shaping Tumor Biology in Subgroups of Breast Cancer Including TNBC

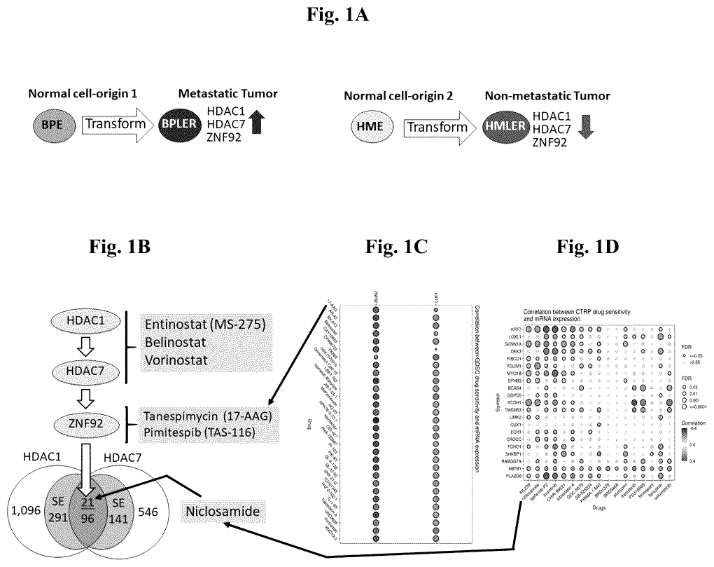

Based on clinical observations, it was hypothesized that the intrinsic properties of the normal cell-origin might be involved in shaping tumor biology in other subgroups of breast cancer such as TNBC^{[2]}. To test this hypothesis, identical genetic elements were used to transform two different ER-negative normal breast cell-origins isolated from the same donor^{[2]}. While one normal cell-origin developed invasive and metastatic TNBC, the other normal cell-origin developed non-metastatic indolent TNBC, confirming our hypothesis^{[2]} (FIG. 1A). Since then, other investigators independently verified this hypothesis in numerous cancers^{[3-15]}. However, these cell-origin signatures have been difficult to translate into mechanistic insights because they involve thousands of genes^{[16-18]}. As such, we had a similar challenge having identified more than 6,000 breast cell-origin associated mRNAs in our metastatic TNBC model.

It was reasoned that examining histone modifications could uncover a mechanism that may propagate the normal cell-origin signature in TNBC^{[19]}. After exploring several candidates^{[20-24]}, HDAC7 and HDAC1 were found to be specifically upregulated in the metastatic TNBC cell lineage that was developed, but not in the isogenic non-metastatic cell lineage^{[25]}. Next, it was determined that in these cells HDAC1 is upstream of HDAC7, and these two HDACs co-regulate 1,243 mRNAs in the metastatic TNBC cells^{[26]}. Finally, it was found that approximately 10% of the HDAC1&7 upregulated (n=125) mRNAs are increased via super-enhancers in metastatic TNBC^{[27]}, but not in non-metastatic TNBC^{[25]}. Further, a 60 gene mRNA subset of this cell-origin signature predicted remarkably shorter breast cancer survival, 8.7 years overall and 6.2 years relapse free respectively^{[26]}.

Next, it was discovered that twenty-nine genes that are co-upregulated by HDAC1&7 in metastatic TNBC have a ZNF92 binding site in their promoter^{[26]}. ZNF92 is a primate-specific member of the KRAB-ZNF (Kruppel-associated box domain zinc finger) family with more than 700 genes encoding tissue-specific transcription suppressors. ZNFs have been an understudied protein family (534 papers in PubMed for 700 genes). Even among this family, ZNF92 is perhaps an exceptionally unexplored protein since its cloning in 1991, as it was only mentioned once among 121 genes that are altered with cholesterol-lowering drug atorvastatin in the liver cell line HepG2^{[28]}. Previous to our study, ZNF92 had not been examined in any cancer.

It was inferred that the metastatic TNBC cell-origin related HDAC1-HDAC7-ZNF92 axis could be targeted with a combination of small molecules. As a proof-of-concept, a combination of an anti-worm drug, an antibiotic that inhibits HSP90, and a histone deacetylase inhibitor can synergistically inhibit proliferation of sixteen out of seventeen TNBC and IBC cell lines (FIG. 1B). The repurposing of these clinically approved drugs provides an opportunity to develop a cell-targeted therapy for TNBC and IBC.

### Example 3: Three-Drug Synergy

To illustrate the three-drug synergy, low concentrations of HDAC-I inhibitor, HSP90-I inhibitor, and Niclosamide were used in the experiments. Nevertheless, up to 98% reduction in TNBC cell proliferation was observed, suggesting that it might be possible to achieve meaningful results with clinically recommended doses of Niclosamide (2000 mg/day, 220 mg/m2)[35, 60], Vorinostat (400 mg)[61], Pimitespib (160 mg/day)[62], and Belinostat (1,000 mg/m2/day)[44, 53]. At these doses, the side effects of HDAC-1 include diarrhea, fatigue, pyrexia, nausea, thrombocytopenia, and anorexia. While Niclosamide side effects are generally non-overlapping (itching, drowsiness, dizziness, and skin rash); the side effects of HSP90 inhibitor Pimitespib partially overlap (diarrhea, decreased appetite, malaise, renal impairment, and anemia). Whether any of these toxicities would be exacerbated or other toxicities would emerge with a three-drug combination remains to be seen. However, in the present experiments Entinostat (100 nM), Niclosamide (100 nM), Tanespimycin (50 nM), Belinostat (1 uM), Vorinostat (1 uM), and Pimitespib (0.2-0.6 uM) were used at doses that are 10 to 1000-fold lower than the peak serum concentrations achieved in different clinical trials for Entinostat (1 uM), Niclosamide (2 uM), Tanespimycin (15 uM), Belinostat (200 uM), Vorinostat (40 M), and Pimitespib (7 uM). Therefore, it may be possible to manage these side effects by lowering the dose of some drugs when used in combination. Moreover, two recent studies reported development of novel dual HDAC and HSP90 inhibitors, where a single small molecule inhibited both HDAC and HSP90 that were tested on age-related macular degeneration [63] and leukemia [64].

The concept of targeted therapy has been often mentioned for a class of drugs that target tumor specific gene alterations. While these gene-targeted therapies have been transformative for HER2+ breast cancer, other breast cancer subtypes do not necessarily present such a straightforward gene-targeted therapy opportunity due to heterogeneity and clonal evolution. It was recently reported that the cell-of-origin patterns dominate the molecular classification of 10,000 tumors from 33 types of cancer [65]. The concept of cell-of-origin was expanded to targeted therapy because a substantial portion of the normal cell-origin mRNA, protein and DNA methylation signatures are retained in the breast cancer [2, 11, 12, 66, 67], and these cell-origin signatures can be associated with clinically relevant tumor features such as metastasis [2, 26]. Thus, it was postulated that such a persistent cell-origin signature can be targeted with drug combinations. It is worth mentioning that HDAC1-HDAC7-ZNF92 axis and their 29 downstream targets (ET-29) are rarely mutated in human breast cancers, consistent with a cell-of-origin signature (FIG. 8 and Table 4). As a proof-of-concept, this metastatic breast cancer cell-of-origin signature was shown to be synergistically targeted with HDAC and HSP90 inhibitors combined with Niclosamide in sixteen out of seventeen breast cancer cell lines.

TABLE 4
The frequency of genomic alterations in the cell-of-origin signature.
Frequency of genetic alterations in the cell-of-origin
signature genes

| | Gene | Number of | |
|---|---|---|---|
| | Symbol | Samples Altered | Percent Samples Altered |
| 1 | CUX1 | 14 | 0.40% |
| 2 | HDAC1 | 10 | 0.30% |
| 3 | CROCC | 10 | 0.30% |
| 4 | EPHB3 | 10 | 0.30% |
| 5 | PCDH1 | 10 | 0.30% |
| 6 | FCHO1 | 9 | 0.30% |
| 7 | PLA2G6 | 7 | 0.20% |
| 8 | MYO1B | 5 | 0.20% |
| 9 | LIMK2 | 4 | 0.10% |
| 10 | SCNN1A | 4 | 0.10% |
| 11 | DKK3 | 3 | 0.10% |
| 12 | FIBCD1 | 3 | 0.10% |
| 13 | GDPD5 | 3 | 0.10% |
| 14 | KRT7 | 3 | 0.10% |
| 15 | SHKBP1 | 3 | 0.10% |
| 16 | ZNF92 | 2 | 0.10% |
| 17 | ABTB1 | 2 | 0.10% |
| 18 | PDLIM1 | 2 | 0.10% |
| 19 | RABGGTA | 2 | 0.10% |
| 20 | TMEM53 | 2 | 0.10% |
| 21 | HDAC7 | 1 | 0.00% |
| 22 | ECH1 | 1 | 0.00% |
| 23 | LOXL1 | 1 | 0.00% |
| 24 | BCAS4 | 0 | 0.00% |

Despite their expression ubiquitously in all tissues, hereditary mutations cause tissue-specific cancers [68]. It seems that rather than being a blank canvas the tissue origin is an active partner in the development of familial tumors[69]. Conversely, because the mutational spectrum of sporadic tumors seems less tissue-specific, it has been proposed that sporadic tumor should be classified according molecular alterations regardless of their tissue origin[69]. However, recent basket trials suggest that the response to targeted anti-cancer drug response often depends on the tissue type[69]. Consistent with these observations, the present work suggests that the cell-of-origin is involved in shaping breast tumor phenotype and drug response[70]. Further, it was demonstrate that the cell-of-origin signature can be leveraged to develop cell-targeted therapies.

### Example 4: Therapies

The Gene Set Cancer Analysis (GSCA) online platformn^{[30]} was utilized to discover that ZNF92 expression is uniquely associated with sensitivity to the 90-kDa heat shock protein (Hsp90) inhibitor Tanespimycin (17-AAG), a synthetic analog of the antibiotic Geldanamycin^{[33]}. HSP90 is a molecular chaperone that regulates protein hemostasis by regulating the folding, stabilization, activation, and degradation of over 400 proteins^{[34]}.

The GSCA includes the Genomics of Drug Sensitivity in Cancer (GDSC) dataset that contains IC50 of 265 small molecules in 860 cell lines and their corresponding mRNA expression^{[31]}. Significantly, 17-AAG was the only hit for ZNF92 in the GDSC, as all the other top 30 hits are anti-correlated, meaning that ZNF92 expression predicted resistance to these drugs (FIG. 1C).

Using the Genomics of Therapeutics Response Portal (CTRP)^{[30, 32]}, the 29 gene mRNA signature downstream of the HDAC1-HDAC7-ZNF92 axis (Table 2, reproduced below) was found to correlate with net sensitivity to 9/481 small molecules in 1001 cell lines.

TABLE 2
The 29 gene mRNA signature downstream of the HDACI-HDAC7-ZNF92 axis.

| | Entrez ID | Gene Symbol | Gene Name |
|---|---|---|---|
| 1 | 80325 | ABTB1 | ankyrin repeat and BTB domain containing 1 |
| 2 | 9289 | ADGRG1 | adhesion G protein-coupled receptor G1 |
| 3 | 113451 | AZIN2 | antizyme inhibitor 2 |
| 4 | 55653 | BCAS4 | breast carcinoma amplified sequence 4 |
| 5 | 79934 | COQ8B | coenzyme Q8B |
| 6 | 9696 | CROCC | ciliary rootlet coiled-coil, rootletin |
| 7 | 1523 | CUX1 | cut like homeobox 1 |
| 8 | 27122 | DKK3 | dickkopf WNT signaling pathway inhibitor 3 |
| 9 | 1891 | ECH1 | enoyl-CoA hydratase 1 |
| 10 | 2049 | EPHB3 | EPH receptor B3 |
| 11 | 23149 | FCHO1 | FCH and mu domain containing endocytic adaptor 1 |
| 12 | 84929 | FIBCD1 | fibrinogen C domain containing 1 |
| 13 | 81544 | GDPD5 | glycerophosphodiester phosphodiesterase domain |
| 14 | 3855 | KRT7 | keratin 7 |
| 15 | 3985 | LIMK2 | LIM domain kinase 2 |
| 16 | 114783 | LMTK3 | lemur tyrosine kinase 3 |
| 17 | 4016 | LOXL1 | lysyl oxidase like 1 |
| 18 | 4430 | MYO1B | myosin IB |
| 19 | 5097 | PCDH1 | protocadherin 1 |
| 20 | 9124 | PDLIM1 | PDZ and LIM domain 1 |
| 21 | 8398 | PLA2G6 | phospholipase A2 group VI |
| 22 | 5875 | RABGGTA | Rab geranylgeranyltransferase subunit alpha |
| 23 | 6337 | SCNN1A | sodium channel epithelial 1 subunit alpha |
| 24 | 92799 | SHKBP1 | SH3KBP1 binding protein 1 |
| 25 | S5315 | SLC29A3 | solute carrier family 29 member 3 |
| 26 | 56848 | SPHK2 | sphingosine kinase 2 |
| 27 | 79816 | TLE6 | TLE family member 6, subcortical maternal |
| 28 | 79639 | TMEM53 | transmembrane protein 53 |
| 29 | 641649 | TMEM91 | transmembrane protein 91 |

The top nine Clinical Trials Reporting Program (CTRP) hits in rank order include ML239 (breast cancer stem cell inhibitor), Niclosamide (anti tapeworm drug), Tipifamib (H-Ras inhibitor), Tivantinib (MET inhibitor), CHIR-99021 (GSK-3α/β inhibitor), Tubastatin-A (HDAC6 inhibitor), GDC-0879 (B-Raf inhibitor), SB-525334 (transforming growth factor β1 receptor (ALK5) inhibitor, PRIMA-1-met (p53 re-activation and induction of massive apoptosis) (FIG. 11D). Among these, only two were approved by the U.S. Food and Drug Administration (FDA); Niclosamide was approved by the FDA to treat tapeworm infection in 1815, and Tipifarmib recently received a breakthrough therapy designation by the FDA for the treatment of patients with recurrent or metastatic HRAS mutant head and neck squamous cell carcinoma (HNSCC)^{[36]}. Since Niclosamide was the highest ranked FDA approved drug it was selected for the combination experiments.

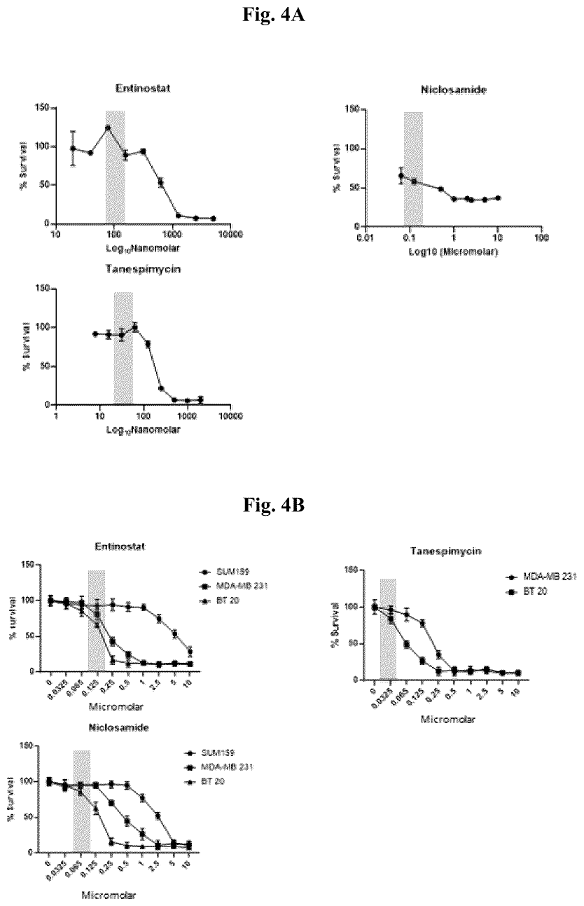

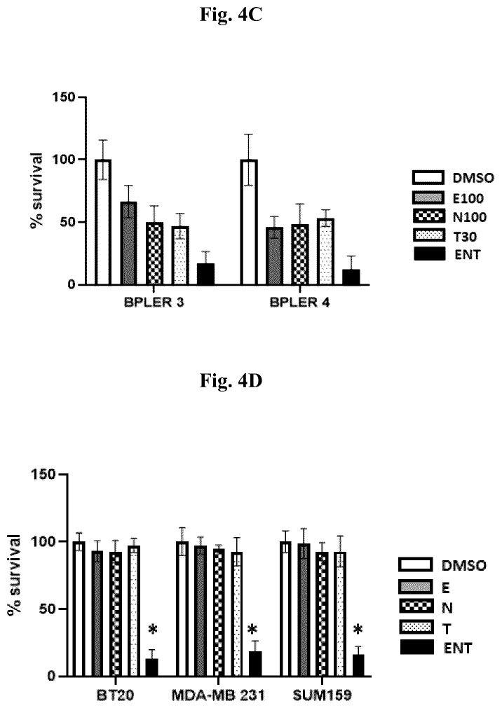

As was previously shown that Entinostat (MS-275) inhibits both HDAC1 and HDAC7^{[37]}, it was reasoned that combining Entinostat (E), with Tanespimycin (T) and Niclosamide (N) may target all the components of the HDAC1-HDAC7-ZNF92 axis. First, dose-response experiments were carried out in several TNBC cell lines and the drug concentrations that have minimal effect on cell proliferation (FIG. 4A-4C) were determined. Encouragingly, while these low doses reduced cell proliferation only by 16% individually, the three-drug combination resulted in over 84% average inhibition of proliferation, consistent with a synergistic activity (FIG. 4D). These results were confirmed in multiple TNBC cell lines, where the observed average inhibition was significantly greater than expected by additive activity (FIG. 2). In several TNBC cell lines, there was exceptional sensitivity to Niclosamide (HCC1913, MFM223) or Tanespimycin (Cal148) (FIG. 2).

These results provided a proof-of-concept that HDAC and HSP90 inhibitors can be combined with a tape-worm drug Niclosamide to achieve synergistic effects in TNBC. Entinostat is still undergoing Phase H-III trials and has not been FDA approved yet. Moreover, even though Tanespimycin concentrations required for activity in preclinical models could be safely achieved in patient plasma in phase 1/2 clinical trials, it was reported that the manufacturer of Tanespimycin declined advancing it to phase III trials.

To potentially translate the findings to the clinic more rapidly, clinically approved alternatives for Entinostat and Tanespimycin were determined. Currently, there are five FDA approved HDAC inhibitors (HDAC-I) available for cancer treatment: Vorinostat (SAHA), Belinostat (PXD-101), Romidepsin (FK-228), Epidaza (HBI-8000) and Panobinostat (LBH589)^{[38]}. Furthermore, a new orally available HSP90 inhibitor TAS-116 (Pimitespib), was recently approved to treat gastrointestinal stromal tumors that have progressed after chemotherapy in Japan^{[39]}.

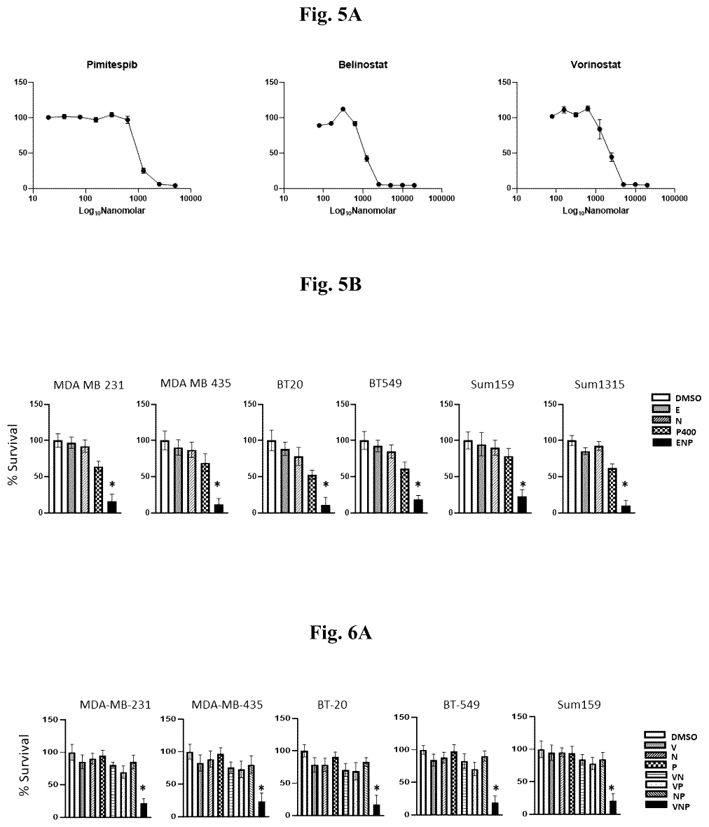

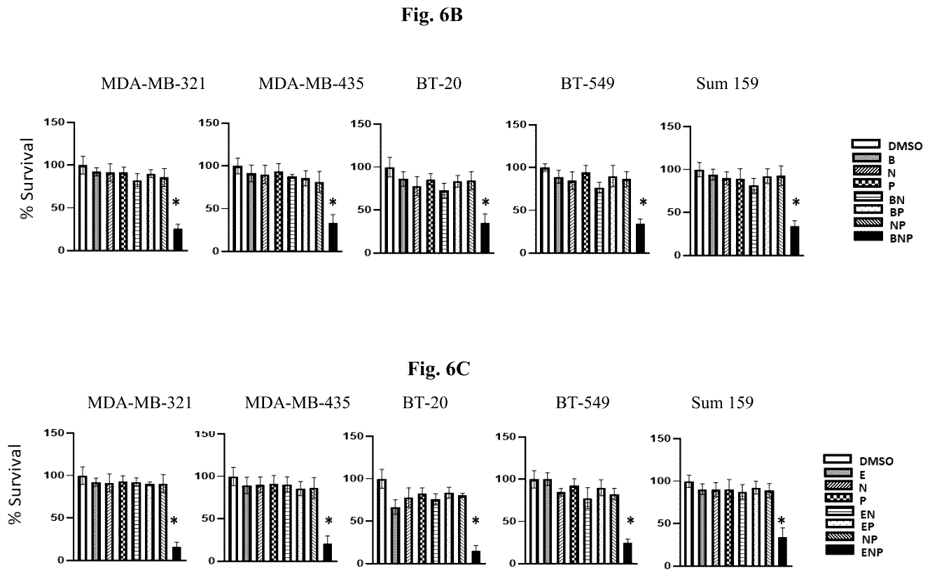

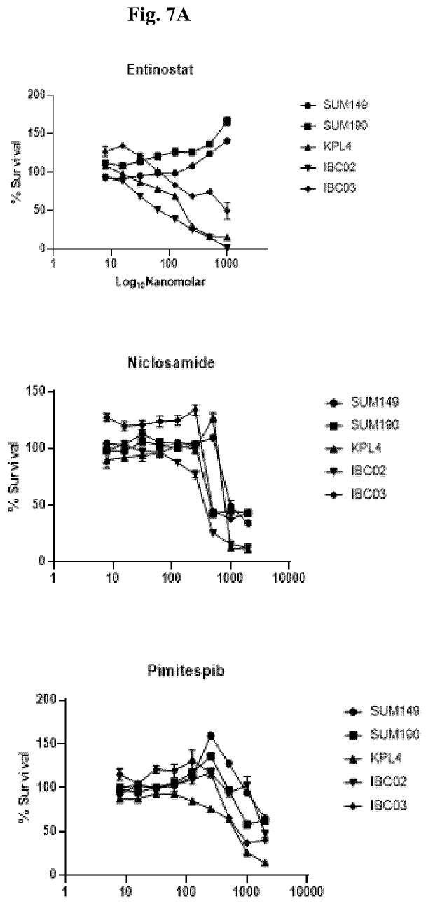

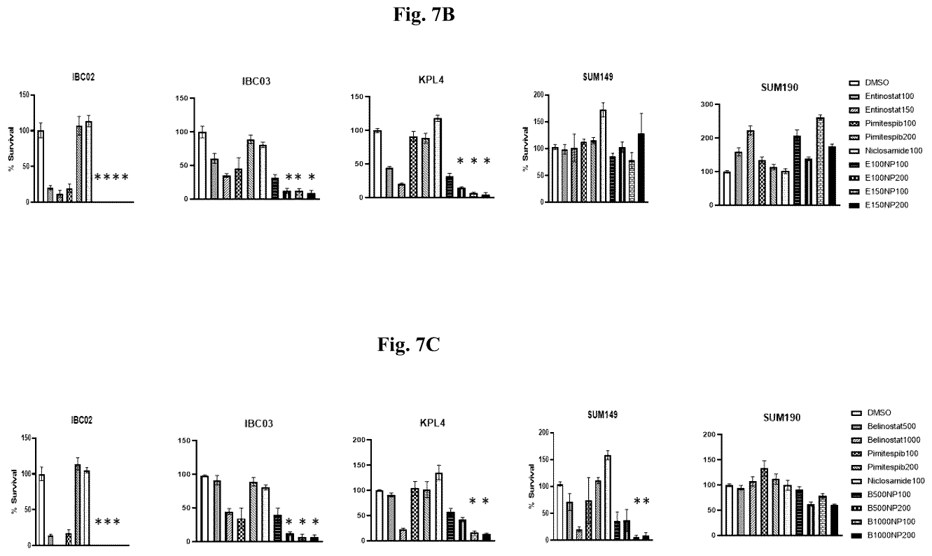

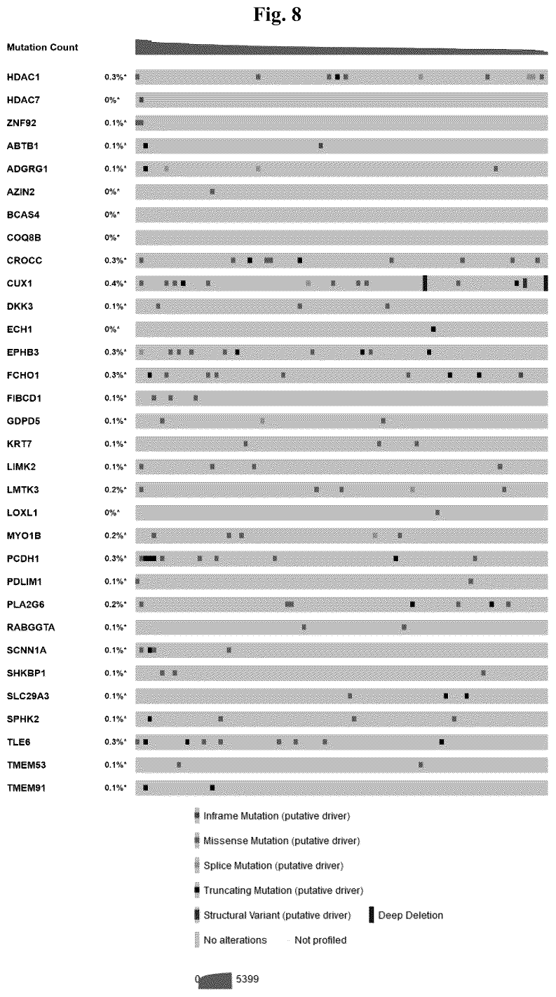

By way of single drug dose-response experiments the minimally effective doses of Pimitespib (P), Vorinostat (V), and Belinostat (B) (FIG. 5A) were determined. It was found that it was possible to replace one HSP90 inhibitor (HSP90-I) Tanespimycin with another HSP90-L, Pimitespib, in combination with Entinostat and Niclosamide with similar synergistic results (FIG. 5B). Next it was showed that HDAC inhibitor Entinostat could be replaced by two different HDAC-I Vorinostat (V) and Belinostat (B) in combination with Niclosamide (N) and Pimitespib (P) in five out of five TNBC cell lines tested. In these experiments it was expected approximately 20% to 30% inhibition of cell proliferation if these three drugs acted additively. In contrast, approximately 70 to 80% inhibition was observed consistent with synergistic action (FIG. 6A-6C). The ability to swap different small molecules for the same target suggests that the observed synergies are probably due to bonafide target synergies and not the result of an idiosyncratic drug interaction.

Next, it was determined the lowest combined doses that can produce a near complete inhibition of TNBC proliferation, and determined that 400-600 nM Pimitespib combined with 100 nM Niclosamide and 1,000 nM Vorinostat produced 96-98% inhibition of proliferation in three out of four TNBC cell lines (FIG. 3A), which was replicated with another HDAC inhibitor Belinostat (FIG. 3B). It is worth pointing out that 10 to 200-fold higher concentrations of Vorinostat, Belinostat, Pimitespib and Niclosamide are achievable in the clinic (Table 5)^{[39-46]}. Therefore, it may be possible to eliminate tumor cells with a three-drug combination at clinically relevant doses.

TABLE 5
Comparison of the peak serum concentrations achieved
for HDAC inhibitors, HSP90 inhibitors and Niclosamide,
and the drug concentrations used in this study.

| | Phase I-II | Experimental | |
|---|---|---|---|
| | Cₘₐₓ | three-drug | |
| Drug | (nM) | (nM) | Ratio |
| Entinostat | 1,000 | 100 | 10 |
| Vorinostat | 40,000 | 1,000 | 40 |
| Belinostat | 200,000 | 1,000 | 200 |
| Niclosamide | 2,000 | 100 | 20 |
| Tanespimycin | 15,000 | 50 | 300 |
| Pimitespib | 7,000 | 200-600 | 11-35 |

Lastly, it was previously showed that HDAC inhibitors inhibited self-renewal of IBC tumor spheroids and tumor emboli^{[47, 48]}. Significantly, it was found that four out of five IBC cell lines (IBC02, IBC03, KPL4 and SUM-149) are sensitive to both EPN and BPN triple-drug combinations (Table 6).

TABLE 6
Survival of inflammatory breast cancer cells with HDAC-I,
HSP90-I and niclosamide.

| | IBC02 | IBC03 | KPL4 | SUM149 | SUM190 |
|---|---|---|---|---|---|
| DMSO | 101 | 100 | 100 | 103 | 100 |
| Entinostat 100 | 21 | 61 | 45 | 99 | 159 |
| Entinonostat150 | 12 | 35 | 20 | 102 | 223 |
| Niclosamide100 | 20 | 46 | 91 | 113 | 135 |
| Pimitespib100 | 107 | 89 | 89 | 115 | 114 |
| Pimitespib200 | 114 | 81 | 118 | 172 | 102 |
| E100N100P100 | 0 | 32 | 32 | 86 | 208 |
| Expected Additive | 4 | 25 | 36 | 128 | 244 |
| E100N100P200 | 0 | 12 | 18 | 103 | 139 |
| Expected Additive | 3 | 14 | 48 | 192 | 218 |
| E150N100P100 | 0 | 13 | 7 | 78 | 262 |
| Expected Additive | 5 | 23 | 16 | 132 | 342 |
| E150N100P200 | 0 | 9 | 5 | 128 | 177 |
| Expected Additive | 3 | 13 | 22 | 197 | 306 |
| DMSO | 100 | 99 | 100 | 105 | 100 |
| Belinostat500 | 14 | 91 | 91 | 71 | 95 |
| Belinostat1000 | 0 | 45 | 23 | 21 | 107 |
| Niclosamide100 | 17 | 35 | 104 | 74 | 133 |
| Pimitespib100 | 114 | 89 | 102 | 111 | 112 |
| Pimitespib200 | 105 | 81 | 136 | 158 | 100 |
| B500N100P100 | 0 | 40 | 58 | 37 | 91 |
| Expected Additive | 3 | 28 | 97 | 59 | 142 |
| B1000N100P100 | 0 | 12 | 42 | 37 | 62 |
| Expected Additive | 0 | 14 | 25 | 17 | 160 |
| B500N100P200 | 0 | 7 | 17 | 6 | 79 |
| Expected Additive | 3 | 26 | 130 | 83 | 126 |
| B1000N100P200 | 0 | 2 | 15 | 8 | 60 |
| Expected Additive | 0 | 13 | 33 | 24 | 143 |

### REFERENCES

1) Epidemiology of inflammatory breast cancer (IBC). Anderson, W. F., C. Schairer, B. E. Chen, K. W. Hance, and P. H. Levine, Breast Dis. 22: p. 9-23, 2005.
2) Transformation of different human breast epithelial cell types leads to distinct tumor phenotypes. Ince, T. A., A. L. Richardson, G. W. Bell, M. Saitoh, S. Godar, A. E. Karnoub, J. D. Iglehart, and R. A. Weinberg, Cancer Cell. 12(2): p. 160-70, 2007.
3) Cell type of origin as well as genetic alterations contribute to breast cancer phenotypes. Bhagirath, D., X. Zhao, W. W. West, F. Qiu, H. Band, and V. Band, Oncotarget. 6(11): p. 9018-30, 2015.
4) Immortalization of Different Breast Epithelial Cell Types Results in Distinct Mitochondrial Mutagenesis. Kwon, S., S. S. Kim, H. E. Nebeck, and E. H. Ahn, Int J Mol Sci. 20(11), 2019.
5) Mammary Precancerous Stem and Non-Stem Cells Evolve into Cancers of Distinct Subtypes. Bu, W., Z. Liu, W. Jiang, C. Nagi, S. Huang, D. P. Edwards, E. Jo, Q. Mo, C. J. Creighton, S. G. Hilsenbeck, A. D. Leavitt, M. T. Lewis, S. T. C. Wong, and Y. Li, Cancer Res. 79(1): p. 61-71, 2019.
6) Time to Classify Tumours of the Stomach and the Kidneys According to Cell of Origin. Waldum, H. and P. Mjones, Int J Mol Sci. 22(24), 2021.
7) Anatomic position determines oncogenic specificity in melanoma. Weiss, J. M., M. V. Hunter, N. M. Cruz, A. Baggiolini, M. Tagore, Y. Ma, S. Misale, M. Marasco, T. Simon-Vermot, N. R. Campbell, F. Newell, J. S. Wilmott, P. A. Johansson, J. F. Thompson, G. V. Long, J. V. Pearson, G. J. Mann, R. A. Scolyer, N. Waddell, E. D. Montal, T. H. Huang, P. Jonsson, M. T. A. Donoghue, C. C. Harris, B. S. Taylor, T. Xu, R. Chaligne, P. V. Shliaha, R. Hendrickson, A. A. Jungbluth, C. Lezcano, R. Koche, L. Studer, C. E. Ariyan, D. B. Solit, J. D. Wolchok, T. Merghoub, N. Rosen, N. K. Hayward, and R. M. White, Nature. 604(7905): p. 354-361, 2022.
8) Cell-of-origin determined by both gene expression profiling and immunohistochemistry is the strongest predictor of survival in patients with diffuse large B-cell lymphoma. Abdulla, M., P. Hollander, T. Pandzic, L. Mansouri, S. B. Ednersson, P. O. Andersson, M. Hultdin, M. Fors, M. Erlanson, S. Degerman, H. M. Petersen, F. Asmar, K. Gronbaek, G. Enblad, L. Cavelier, R. Rosenquist, and R. M. Amini, Am J Hematol. 95(1): p. 57-67, 2020.
9) Cell of origin markers identify different prognostic subgroups of lung adenocarcinoma. Tabbo, F., A. Nottegar, F. Guerrera, E. Migliore, C. Luchini, F. Maletta, N. Veronese, L. Montagna, M. Gaudiano, F. Di Giacomo, P. L. Filosso, L. Delsedime, G. Ciccone, A. Scarpa, A. Sapino, A. Oliaro, E. Ruffini, G. Inghirami, and M. Chilosi, Hum Pathol. 75: p. 167-178, 2018.
10) Cell of Origin Influences Pancreatic Cancer Subtype. Flowers, B. M., H. Xu, A. S. Mulligan, K. J. Hanson, J. A. Seoane, H. Vogel, C. Curtis, L. D. Wood, and L. D. Attardi, Cancer Discov. 11(3): p. 660-677, 2021.
11) Taxonomy of breast cancer based on normal cell phenotype predicts outcome. Santagata, S., A. Thakkar, A. Ergonul, B. Wang, T. Woo, R. Hu, J. C. Harrell, G. McNamara, M. Schwede, A. C. Culhane, D. Kindelberger, S. Rodig, A. Richardson, S. J. Schnitt, R. M. Tamimi, and T. A. Ince, J Clin Invest. 124(2): p. 859-70, 2014.
12) Normal cell phenotypes of breast epithelial cells provide the foundation of a breast cancer taxonomy. Santagata, S. and T. A. Ince, Expert Rev Anticancer Ther. 14(12): p. 1385-9, 2014.
13) Gene expression signature of normal cell-of-origin predicts ovarian tumor outcomes. Merritt, M. A., S. Bentink, M. Schwede, M. P. Iwanicki, J. Quackenbush, T. Woo, E. S. Agoston, F. Reinhardt, C. P. Crum, R. S. Berkowitz, S. C. Mok, A. E. Witt, M. A. Jones, B. Wang, and T. A. Ince, PLoS One. 8(11): p. e80314, 2013.
14) Cell-specific effects of RB or RB/p107 loss on retinal development implicate an intrinsically death-resistant cell-of-origin in retinoblastoma. Chen, D., I. Livne-bar, J. L. Vanderluit, R. S. Slack, M. Agochiya, and R. Bremner, Cancer Cell. 5(6): p. 539-51, 2004.
15) Pan-cancer methylome analysis for cancer diagnosis and classification of cancer cell of origin. Shimizu, D., K. Taniue, Y. Matsui, H. Haeno, H. Araki, F. Miura, M. Fukunaga, K. Shiraishi, Y. Miyamoto, S. Tsukamoto, A. Komine, Y. Kobayashi, A. Kitagawa, Y. Yoshikawa, K. Sato, T. Saito, S. Ito, T. Masuda, A. Niida, M. Suzuki, H. Baba, T. Ito, N. Akimitsu, Y. Kodera, and K. Mimori, Cancer Gene Ther. 29(5): p. 428-436, 2022.
16) Epigenetic signatures in cancer: proper controls, current challenges and the potential for clinical translation. Mancarella, D. and C. Plass, Genome Med. 13(1): p. 23, 2021.
17) Distinct epigenomic landscapes of pluripotent and lineage-committed human cells. Hawkins, R. D., G. C. Hon, L. K. Lee, Q. Ngo, R. Lister, M. Pelizzola, L. E. Edsall, S. Kuan, Y. Luu, S. Klugman, J. Antosiewicz-Bourget, Z. Ye, C. Espinoza, S. Agarwahl, L. Shen, V. Ruotti, W. Wang, R. Stewart, J. A. Thomson, J. R. Ecker, and B. Ren, Cell Stem Cell. 6(5): p. 479-91, 2010.
18) Cells of Origin for Prostate Cancer. Xin, L., Adv Exp Med Biol. 1210: p. 67-86, 2019.
19) Epigenetics decouples mutational from environmental robustness. Did it also facilitate multicellularity? Gombar, S., T. MacCarthy, and A. Bergman, PLoS Comput Biol. 10(3): p. e1003450, 2014.
20) Growth-inhibitory and tumor-suppressive functions of p53 depend on its repression of CD44 expression. Godar, S., T. A. Ince, G. W. Bell, D. Feldser, J. L. Donaher, J. Bergh, A. Liu, K. Miu, R. S. Watnick, F. Reinhardt, S. S. McAllister, T. Jacks, and R. A. Weinberg, Cell. 134(1): p. 62-73, 2008.
21) Systemic endocrine instigation of indolent tumor growth requires osteopontin. McAllister, S. S., A. M. Gifford, A. L. Greiner, S. P. Kelleher, M. P. Saelzler, T. A. Ince, F. Reinhardt, L. N. Harris, B. L. Hylander, E. A. Repasky, and R. A. Weinberg, Cell. 133(6): p. 994-1005, 2008.
22) HSF1 drives a transcriptional program distinct from heat shock to support highly malignant human cancers. Mendillo, M. L., S. Santagata, M. Koeva, G. W. Bell, R. Hu, R. M. Tamimi, E. Fraenkel, T. A. Ince, L. Whitesell, and S. Lindquist, Cell. 150(3): p. 549-62, 2012.
23) A Genome-wide siRNA Screen Identifies Proteasome Addiction as a Vulnerability of Basal-like Triple-Negative Breast Cancer Cells. Petrocca, F., G. Altschuler, S. Tan, M. Mendillo, H. Yan, D. J. Jerry, A. Kung, W. Hide, T. Ince, and J. Lieberman, Cancer Cell. 24(2): p. 182-196, 2013.
24) High levels of nuclear heat-shock factor 1 (HSF1) are associated with poor prognosis in breast cancer. Santagata, S., R. Hu, N. U. Lin, M. L. Mendillo, L. C. Collins, S. E. Hankinson, S. J. Schnitt, L. Whitesell, R. M. Tamimi, S. Lindquist, and T. A. Ince, Proc Natl Acad Sci USA. 108(45): p. 18378-83, 2011.
25) Identification of a cancer stem cell-specific function for the histone deacetylases, HDAC1 and HDAC7, in breast and ovarian cancer. Witt, A. E., C. W. Lee, T. I. Lee, D. J. Azzam, B. Wang, C. Caslini, F. Petrocca, J. Grosso, M. Jones, E. B. Cohick, A. B. Gropper, C. Wahlestedt, A. L. Richardson, R. Shiekhattar, R. A. Young, and T. A. Ince, Oncogene. 36(12): p. 1707-1720, 2017.
26) ZNF92, an unexplored transcription factor with remarkably distinct breast cancer over-expression associated with prognosis and cell-of-origin. Kamran, M., U. Bhattacharya, M. Omar, L. Marchionni, and T. A. Ince, NPJ Breast Cancer. 8(1): p. 99, 2022.
27) HDAC7 regulates histone 3 lysine 27 acetylation and transcriptional activity at super-enhancer-associated genes in breast cancer stem cells. Caslini, C., S. Hong, Y. J. Ban, X. S. Chen, and T. A. Ince, Oncogene. 38(39): p. 6599-6614, 2019.
28) RNA-sequencing analysis of HepG2 cells treated with atorvastatin. Stormo, C., M. K. Kringen, R. Lyle, O. K. Olstad, D. Sachse, J. P. Berg, and A. P. Piehler, PLoS One. 9(8): p. e105836, 2014.
29) Vitamin D and androgen receptor-targeted therapy for triple-negative breast cancer. Thakkar, A., B. Wang, M. Picon-Ruiz, P. Buchwald, and T. A. Ince, Breast cancer research and treatment. 157(1); p. 77-90, 2016.
30) GSCALite: a web server for gene set cancer analysis. Liu, C. J., F. F. Hu, M. X. Xia, L. Han, Q. Zhang, and A. Y. Guo, Bioinformatics. 34(21): p. 3771-3772, 2018.
31) Genomics of Drug Sensitivity in Cancer (GDSC): a resource for therapeutic biomarker discovery in cancer cells. Yang, W., J. Soares, P. Greninger, E. J. Edelman, H. Lightfoot, S. Forbes, N. Bindal, D. Beare, J. A. Smith, I. R. Thompson, S. Ramaswamy, P. A. Futreal, D. A. Haber, M. R. Stratton, C. Benes, U. McDermott, and M. J. Garnett, Nucleic Acids Res. 41 (Database issue): p. D955-61, 2013.
32) An interactive resource to identify cancer genetic and lineage dependencies targeted by small molecules. Basu, A., N. E. Bodycombe, J. H. Cheah, E. V. Price, K. Liu, G. I. Schaefer, R. Y. Ebright, M. L. Stewart, D. Ito, S. Wang, A. L. Bracha, T. Liefeld, M. Wawer, J. C. Gilbert, A. J. Wilson, N. Stransky, G. V. Kryukov, V. Dancik, J. Barretina, L. A. Garraway, C. S. Hon, B. Munoz, J. A. Bittker, B. R. Stockwell, D. Khabele, A. M. Stem, P. A. Clemons, A. F. Shamji, and S. L. Schreiber, Cell. 154(5): p. 1151-1161, 2013.
33) HSP90 inhibition is effective in breast cancer: a phase II trial of tanespimycin (17-AAG) plus trastuzumab in patients with HER2-positive metastatic breast cancer progressing on trastuzumab. Modi, S., A. Stopeck, H. Linden, D. Solit, S. Chandarlapaty, N. Rosen, G. D'Andrea, M. Dickler, M. E. Moynahan, S. Sugarman, W. Ma, S. Patil, L. Norton, A. L. Hannah, and C. Hudis, Clin Cancer Res. 17(15): p. 5132-9, 2011.
34) Old and New Approaches to Target the Hsp90 Chaperone. Sanchez, J., T. R. Carter, M. S. Cohen, and B. S. J. Blagg, Curr Cancer Drug Targets. 20(4): p. 253-270, 2020.
35) Niclosamide therapy for tapeworm infections. Pearson, R. D. and E. L. Hewlett, Ann Intern Med. 102(4): p. 550-1, 1985.
36) Tipifarnib in Head and Neck Squamous Cell Carcinoma With HRAS Mutations. Ho, A. L., I. Brana, R. Haddad, J. Bauman, K. Bible, S. Oosting, D. J. Wong, M. J. Ahn, V. Boni, C. Even, J. Fayette, M. J. Flor, K. Harrington, S. B. Kim, L. Licitra, I. Nixon, N. F. Saba, S. Hackenberg, P. Specenier, F. Worden, B. Balsara, M. Leoni, B. Martell, C. Scholz, and A. Gualberto, J Clin Oncol. 39(17): p. 1856-1864, 2021.
37) Identification of a cancer stem cell-specific function for the histone deacetylases, HDAC1 and HDAC7, in breast and ovarian cancer. Witt, A., C. Lee, T. Lee, D. Azzam, B. Wang, C. Caslini, F. Petrocca, J. Grosso, M. Jones, and E. Cohick, Oncogene. 36(12): p. 1707-1720, 2017.
38) Quinazoline Based HDAC Dual Inhibitors as Potential Anti-Cancer Agents. Dhuguru, J. and O. A. Ghoneim, Molecules. 27(7), 2022.
39) Pimitespib in patients with advanced gastrointestinal stromal tumor (CHAPTER-GIST-301): a randomized, double-blind, placebo-controlled phase III trial. Kurokawa, Y., Y. Honma, A. Sawaki, Y. Naito, S. Iwagami, Y. Komatsu, T. Takahashi, T. Nishida, and T. Doi, Ann Oncol. 33(9): p. 959-967, 2022.
40) Phase 11 trial to investigate the safety and efficacy of orally applied niclosamide in patients with metachronous or synchronous metastases of a colorectal cancer progressing after therapy: the NIKOLO trial. Burock, S., S. Daum, U. Keilholz, K. Neumann, W. Walther, and U. Stein, BMC Cancer. 18(1). p. 297, 2018.
41) Phase Ib trial of reformulated niclosamide with abiraterone/prednisone in men with castration-resistant prostate cancer. Parikh, M., C. Liu, C. Y. Wu, C. P. Evans, M. Dall'Era, D. Robles, P. N. Lara, N. Agarwal, A. C. Gao, and C. X. Pan, Sci Rep. 11(1): p. 6377, 2021.
42) A phase I, pharmacokinetic and pharmacodynamic study on vorinostat in combination with 5-fluorouracil, leucovorin, and oxaliplatin in patients with refractory colorectal cancer. Fakih, M. G., L. Pendyala, G. Fetterly, K. Toth, J. A. Zwiebel, I. Espinoza-Delgado, A. Litwin, Y. M. Rustum, M. E. Ross, J. L. Holleran, and M. J. Egorin, Clin Cancer Res. 15(9): p. 3189-95, 2009.
43) Phase I trial of MS-275, a histone deacetylase inhibitor, administered weekly in refractory solid tumors and lymphoid malignancies. Kummar, S., M. Gutierrez, E. R. Gardner, E. Donovan, K. Hwang, E. J. Chung, M. J. Lee, K. Maynard, M. Kalnitskiy, A. Chen, G. Melillo, Q. C. Ryan, B. Conley, W. D. Figg, J. B. Trepel, J. Zwiebel, J. H. Doroshow, and A. J. Murgo, Clin Cancer Res. 13(18 Pt 1): p. 5411-7, 2007.
44) A phase I study of the safety and pharmacokinetics of the histone deacetylase inhibitor belinostat administered in combination with carboplatin and/or paclitaxel in patients with solid tumours. Lassen, U., L. R. Molife, M. Sorensen, S. A. Engelholm, L. Vidal, R. Sinha, R. T. Penson, P. Buhl-Jensen, E. Crowley, J. Tjornelund, P. Knoblauch, and J. S. de Bono, Br J Cancer. 103(1): p. 12-7, 2010.
45) A phase 1 pharmacokinetic and pharmacodynamic study of the histone deacetylase inhibitor belinostat in patients with advanced solid tumors. Steele, N. L., J. A. Plumb, L. Vidal, J. Tjornelund, P. Knoblauch, A. Rasmussen, C. E. Ooi, P. Buhl-Jensen, R. Brown, T. R. Evans, and J. S. DeBono, Clin Cancer Res. 14(3): p. 804-10, 2008.
46) Phase 1 and pharmacologic study of MS-275, a histone deacetylase inhibitor, in adults with refractory and relapsed acute leukemias. Gojo, I., A. Jiemjit, J. B. Trepel, A. Sparreboom, W. D. Figg, S. Rollins, M. L. Tidwell, J. Greer, E. J. Chung, M. J. Lee, S. D. Gore, E. A. Sausville, J. Zwiebel, and J. E. Karp, Blood. 109(7): p. 2781-90, 2007.
47) Suberoylanilide hydroxamic acid blocks self-renewal and homotypic aggregation of inflammatory breast cancer spheroids. Robertson, F. M., W. A. Woodward, R. Pickei, Z. Ye, W. Bornmann, A. Pal, Z. Peng, C. S. Hall, and M. Cristofanilli, Cancer. 116(11 Suppl): p. 2760-7, 2010.
48) The class I HDAC inhibitor Romidepsin targets inflammatory breast cancer tumor emboli and synergizes with paclitaxel to inhibit metastasis. Robertson, F. M., K. Chu, K. M. Boley, Z. Ye, H. Liu, M. C. Wright, R. Moraes, X. Zhang, T. L. Green, S. H. Barsky, C. Heise, and M. Cristofanilli, J Exp Ther Oncol. 10(3): p. 219-33, 2013.
49) Drug combination and repurposing for cancer therapy: the example of breast cancer. Correia, A. S., F. Gartner, and N. Vale, Heliyon. 7(1): p. e05948, 2021.
50) Combination of trastuzumab and tanespimycin (17-AAG, KOS-953) is safe and active in trastuzumab-refractory HER-2 overexpressing breast cancer: a phase I dose-escalation study. Modi, S., A. T. Stopeck, M. S. Gordon, D. Mendelson, D. B. Solit, R. Bagatell, W. Ma, J. Wheler, N. Rosen, L. Norton, G. F. Cropp, R. G. Johnson, A. L. Hannah, and C. A. Hudis, J Clin Oncol. 25(34): p. 5410-7, 2007.
51) Identification of a Triple Drug Combination That Is Synergistically Cytotoxic for Triple-Negative Breast Cancer Cells Using a Novel Combination Discovery Approach. Ahmad, S., Q. He, K. P. Williams, and J. E. Scott, SLAS Discov. 25(8): p. 923-938, 2020.
52) A Synergistic Combination of Niclosamide and Doxorubicin as an Efficacious Therapy for All Clinical Subtypes of Breast Cancer. Lohiya, G. and D. S. Katti, Cancers (Basel). 13(13), 2021.
53) Belinostat in combination with standard cyclophosphamide, doxorubicin, vincristine and prednisone as first-line treatment for patients with newly diagnosed peripheral T-cell lymphoma. Johnston, P. B., A. F. Cashen, P. G. Nikolinakos, A. W. Beaven, S. K. Barta, G. Bhat, S. J. Hasal, S. De Vos, Y. Oki, C. Deng, and F. M. Foss, Exp Hematol Oncol. 10(1): p. 15, 2021.
54) Combined chemotherapy for triple negative breast cancer treatment by paclitaxel and niclosamide nanocrystals loaded thermosensitive hydrogel. Zhao, D., C. Hu, Q. Fu, and H. Lv, Eur J Pharm Sci. 167: p. 105992, 2021.
55) Drug Repurposing for Triple-Negative Breast Cancer. Avalos-Moreno, M., A. Lopez-Tejada, J. L. Blaya-Canovas, F. E. Cara-Lupianez, A. Gonzalez-Gonzalez, J. A. Lorente, P. Sanchez-Rovira, and S. Granados-Principal, J Pers Med. 10(4), 2020.
56) Synergistic Enhancement of Cancer Therapy Using HDAC Inhibitors: Opportunity for Clinical Trials. Hontecillas-Prieto, L., R. Flores-Campos, A. Silver, E. de Alava, N. Hajji, and D. J. Garcia-Dominguez, Front Genet. 11: p. 578011, 2020.
57) Guided screen for synergistic three-drug combinations. Cokol-Cakmak, M., S. Cetiner, N. Erdem, F. Bakan, and M. Cokol, PLoS One. 15(7): p. e0235929, 2020.
58) Prediction of ultra-high-order antibiotic combinations based on pairwise interactions. Katzir, I., M. Cokol, B. B. Aldridge, and U. Alon, PLoS Comput Biol. 15(1): p. e1006774, 2019.
59) Charting the Fragmented Landscape of Drug Synergy. Meyer, C. T., D. J. Wooten, C. F. Lopez, and V. Quaranta, Trends Pharmacol Sci. 41(4): p. 266-280, 2020.
60) Efficacy of Niclosamide vs Placebo in SARS-CoV-2 Respiratory Viral Clearance, Viral Shedding, and Duration of Symptoms Among Patients With Mild to Moderate COVID-19: A Phase 2 Randomized Clinical Trial. Cairns, D. M., D. Dulko, J. K. Griffiths, Y. Golan, T. Cohen, L. Trinquart, L. L. Price, K. R. Beaulac, and H. P. Selker, JAMA Netw Open. 5(2): p. e2144942, 2022.
61) FDA approval summary: vorinostat for treatment of advanced primary cutaneous T-cell lymphoma. Mann, B. S., J. R. Johnson, M. H. Cohen, R. Justice, and R. Pazdur, Oncologist. 12(10): p. 1247-52, 2007.
62) Efficacy and safety of TAS-116, an oral inhibitor of heat shock protein 90, in patients with metastatic or unresectable gastrointestinal stromal tumour refractory to imatinib, sunitinib and regorafenib: a phase II, single-arm trial. Doi, T., Y. Kurokawa, A. Sawaki, Y. Komatsu, M. Ozaka, T. Takahashi, Y. Naito, S. Ohkubo, and T. Nishida, Eur J Cancer. 121: p. 29-39, 2019.
63) The HDAC/HSP90 Inhibitor G570 Attenuated Blue Light-Induced Cell Migration in RPE Cells and Neovascularization in Mice through Decreased VEGF Production. Hsu, T. J., K. Nepali, C. H. Tsai, Z. Imtiyaz, F. L. Lin, G. Hsiao, M. J. Lai, and Y. W. Cheng, Molecules. 26(14), 2021.
64) A novel dual HDAC and HSP90 inhibitor, MPT0G449, downregulates oncogenic pathways in human acute leukemia in vitro and in vivo. Wu, Y. W., M. W. Chao, H. J. Tu, L. C. Chen, K. C. Hsu, J. P. Liou, C. R. Yang, S. C. Yen, W. C. HuangFu, and S. L. Pan, Oncogenesis. 10(5): p. 39, 2021.
65) Cell-of-Origin Patterns Dominate the Molecular Classification of 10,000 Tumors from 33 Types of Cancer. Hoadley, K. A., C. Yau, T. Hinoue, D. M. Wolf, A. J. Lazar, E. Drill, R. Shen, A. M. Taylor, A. D. Cherniack, V. Thorsson, R. Akbani, R. Bowlby, C. K. Wong, M. Wiznerowicz, F. Sanchez-Vega, A. G. Robertson, B. G. Schneider, M. S. Lawrence, H. Noushmehr, T. M. Malta, N. Cancer Genome Atlas, J. M. Stuart, C. C. Benz, and P. W. Laird, Cell. 173(2): p. 291-304 e6, 2018.
66) Normal Cell-Type Epigenetics and Breast Cancer Classification: A Case Study of Cell Mixture-Adjusted Analysis of DNA Methylation Data from Tumors: Supplementary Issue: Array Platform Modeling and Analysis (A). Houseman, E. A. and T. A. Ince, Cancer informatics. 13: p. CIN. S13980, 2014.
67) Reference-free deconvolution of DNA methylation data and mediation by cell composition effects. Houseman, E. A., M. L. Kile, D. C. Christiani, T. A. Ince, K. T. Kelsey, and C. J. Marsit, BMC bioinformatics. 17(1): p. 1-15, 2016.
68) Tissue-specific tumorigenesis: context matters. Schneider, G., M. Schmidt-Supprian, R. Rad, and D. Saur, Nat Rev Cancer. 17(4): p. 239-253, 2017.
69) Tissue-specificity in cancer: The rule, not the exception. Haigis, K. M., K. Cichowski, and S. J. Elledge, Science. 363(6432): p. 1150-1151, 2019.
70) Not all cancers are created equal: Tissue specificity in cancer genes and pathways. Bianchi, J. J., X. Zhao, J. C. Mays, and T. Davoli, Curr Opin Cell Biol. 63: p. 135-143, 2020.
71) Developing New Analysis Functions for a Translational Research Platform: Extending the cBioPortal for Cancer Genomics. Unberath, P., C. Knell, H. U. Prokosch, and J. Christoph, Stud Health Technol Inform. 258: p. 46-50, 2019.
72) Integrative analysis of complex cancer genomics and clinical profiles using the cBioPortal. Gao, J., B. A. Aksoy, U. Dogrusoz, G. Dresdner, B. Gross, S. O. Sumer, Y. Sun, A. Jacobsen, R. Sinha, E. Larsson, E. Cerami, C. Sander, and N. Schultz, Sci Signal. 6(269): p. pl1, 2013.
73) Developing New Analysis Functions for a Translational Research Platform: Extending the cBioPortal for Cancer Genomics. Unberath, P., C. Knell, H. U. Prokosch, and J. Christoph, Stud Health Technol Inform. 258: p. 46-50, 2019.
74) Integrative analysis of complex cancer genomics and clinical profiles using the cBioPortal. Gao, J., B. A. Aksoy, U. Dogrusoz, G. Dresdner, B. Gross, S. O. Sumer, Y. Sun, A. Jacobsen, R. Sinha, E. Larsson, E. Cerami, C. Sander, and N. Schultz, Sci Signal. 6(269): p. pl1, 2013.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications. The following statements are intended to describe and summarize various features of the invention according to the foregoing description provided in the specification and figures.

### Statements

1. A method comprising:
a. assaying a biological sample from a subject for expression of HDAC1, HDAC7, and ZNF92 genes to determine one or more expression levels for the HDAC1, HDAC7, and ZNF92 genes;
b. comparing the determined expression levels with one or more reference values to identify any altered expression levels in the subject's biological sample, wherein altered expression levels of the HDAC1, HDAC7, and ZNF92 genes in the biological sample relative to the reference value indicates that the subject has cancer with poor prognosis or the subject has malignant cancer, and absence of altered expression of the HDAC1, HDAC7, and ZNF92 genes relative to the reference value indicates that the subject does not have a cancer with poor prognosis or does not have malignant cancer; and optionally
c. administering one or more histone deacetylase inhibitor(s) (HDAC), one or more inhibitor of heat shock protein 90 (HSP90), and one or more helminths to a subject determined to have a cancer with poor prognosis or a malignant cancer.

2. The method of statement 1, wherein the biological sample from the subject is further assayed for expression of one or more genes encoding an amino acid sequence having at least 90% sequence identity to SEQ ID NOs: 3-31.

3. The method of statement 1, wherein the sample is a breast cancer sample.

4. The method of statement 1, wherein the sample is a cervical cancer sample, uterine cancer, or prostate cancer.

5. The method of statement 1, further administering one or more of ML239, Tipifarnib, Tivantinib, CHIR-99021, Tubastatin-A, GDC-0879, SB-525334, PRIMA-1-met, or a combination thereof, to the subject.

6. The method of statement 1, wherein the administering one or more histone deacetylase inhibitor(s), one or more heat shock factor inhibitor, and the one or more helminths are delivered simultaneously.

7. The method of statement 1, wherein the sample is a physiological fluid sample, a tumor tissue sample, or a cancer cell sample.

8. The method of statement 1, wherein the subject is a human.

9. The method of statement 8, wherein the mammal has inflammatory breast cancer.

10. The method of statement 8, wherein the mammal has triple negative breast cancer.

11. The method of statement 1, wherein RNA expression is assayed.

12. The method of statement 1, wherein protein expression is assayed.

13. The method of statement 1, wherein the inhibitor of HSP90 comprises an inhibitor of HSP90a and HSP90s.

14. The method of statement 1, wherein the inhibitor of HSP90 comprises an antibiotic.

15. The method of statement 1, wherein the inhibitor of HSP90 is Pimitespib, Tanespimycin, or a combination thereof.

16. The method of statement 1, wherein the helminth is niclosamide.

17. The method of statement 1, wherein the at least one HDAC inhibitor is Belinostat, Entinostat, Vorinostat, or a combination thereof.

18. A method to prevent, inhibit or treat cancer in a mammal, comprising: administering to the mammal a composition comprising one or more histone deacetylase inhibitor(s), one or more heat shock factor inhibitor, and one or more helminths, wherein the cancer in the mammal is determined to have altered expression levels of HDAC1, HDAC7, and ZNF92 genes, relative to a reference value.

19. The method of statement 18, wherein the biological sample from the subject is further assayed for expression of one or more genes encoding an amino acid sequence having at least 90% sequence identity to SEQ ID NOs: 3-31.

20. The method of statement 18, wherein the mammal is a human.

21. The method of statement 18, wherein the mammal has breast cancer, cervical cancer, uterine cancer, or prostate cancer.

22. A method comprising: (a) contacting cancer cells expressing HDAC1, HDAC7, or ZNF92 genes or producing HDAC1, HDAC7, or ZNF92 proteins with a test agent; (b) measuring HDAC1, HDAC7, or ZNF92 RNA or protein expression levels in the cells or measuring HDAC1, HDAC7, or ZNF92 protein activity levels; and (c) determining that the test agent reduces the expression levels or activity levels of HDAC1, HDAC7, or ZNF92 genes, to thereby identifying a test agent as a candidate agent that reduces HDAC1, HDAC7, or ZNF92 genes expression levels or activity levels.

23. A pharmaceutical composition comprising one or more histone deacetylase inhibitor(s), one or more heat shock factor inhibitor, and one or more helminths.

The specific methods, devices and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

We claim:

1. A method comprising:
a. assaying a biological sample from a subject for expression of HDAC1, HDAC7, and ZNF92 genes to determine one or more expression levels for the HDAC1, HDAC7, and ZNF92 genes;
b. comparing the determined expression levels with one or more reference values to identify any altered expression levels in the subject's biological sample, wherein altered expression levels of the HDAC1, HDAC7, and ZNF92 genes in the biological sample relative to the reference value indicates that the subject has cancer with poor prognosis or the subject has malignant cancer, and absence of altered expression of the HDAC1, HDAC7, and ZNF92 genes relative to the reference value indicates that the subject does not have a cancer with poor prognosis or does not have malignant cancer; and optionally
c. administering one or more histone deacetylase inhibitor(s) (HDAC), one or more inhibitor of heat shock protein 90 (HSP90), and one or more helminths to a subject determined to have a cancer with poor prognosis or a malignant cancer.

2. The method of claim 1, wherein the biological sample from the subject is further assayed for expression of one or more genes encoding an amino acid sequence having at least 90% sequence identity to any of SEQ ID NOs: 3-31.

3. The method of claim 1, wherein the sample is a breast cancer sample.

4. The method of claim 1, wherein the sample is a cervical cancer sample, uterine cancer, or prostate cancer.

5. The method of claim 1, further administering one or more of ML239, Tipifarnib, Tivantinib, CHIR-99021, Tubastatin-A, GDC-0879, SB-525334, PRIMA-1-met, or a combination thereof, to the subject.

6. The method of claim 1, wherein the administering one or more histone deacetylase inhibitor(s), one or more heat shock factor inhibitor, and the one or more helminths are delivered simultaneously.

7. The method of claim 1, wherein the sample is a physiological fluid sample, a tumor tissue sample, or a cancer cell sample.

8. The method of claim 1, wherein the subject is a human.

9. The method of claim 8, wherein the mammal has inflammatory breast cancer.

10. The method of claim 8, wherein the mammal has triple negative breast cancer.

11. The method of claim 1, wherein RNA expression is assayed.

12. The method of claim 1, wherein protein expression is assayed.

13. The method of claim 1, wherein the inhibitor of HSP90 comprises an inhibitor of HSP90a and HSP900.

14. The method of claim 1, wherein the inhibitor of HSP90 comprises an antibiotic.

15. The method of claim 1, wherein the inhibitor of HSP90 is Pimitespib, Tanespimycin, or a combination thereof.

16. The method of claim 1, wherein the helminth is niclosamide.

17. The method of claim 1, wherein the at least one HDAC inhibitor is Belinostat, Entinostat, Vorinostat, or a combination thereof.

18. A method to prevent, inhibit or treat cancer in a mammal, comprising: administering to the mammal a composition comprising one or more histone deacetylase inhibitor(s), one or more heat shock factor inhibitor, and one or more helminths, wherein the cancer in the mammal is determined to have altered expression levels of HDAC1, HDAC7, and ZNF92 genes, relative to a reference value.

19. The method of claim 18, wherein the cancer in the mammal is determined to have altered expression levels of a protein having an amino acid sequence having at least 90% sequence identity to any of SEQ ID NOs: 3-31.

20. The method of claim 18, wherein the mammal is a human.

21. The method of claim 18, wherein the mammal has breast cancer, cervical cancer, uterine cancer, or prostate cancer.

22. A method comprising: (a) contacting cancer cells expressing HDAC1, HDAC7, or ZNF92 genes or producing HDAC1, HDAC7, or ZNF92 proteins with a test agent; (b) measuring HDAC1, HDAC7, or ZNF92 RNA or protein expression levels in the cells or measuring HDAC1, HDAC7, or ZNF92 protein activity levels; and (c) determining that the test agent reduces the expression levels or activity levels of HDAC1, HDAC7, or ZNF92 genes, to thereby identifying a test agent as a candidate agent that reduces HDAC1, HDAC7, or ZNF92 genes expression levels or activity levels.

23. A pharmaceutical composition comprising one or more histone deacetylase inhibitor(s), one or more heat shock factor inhibitor, and one or more helminths.
